SEQUENCE LISTING (3 sequences)

SEQ ID NO: 1 - DNA, 25 bp, synthetic construct
  source 1..25: mol_type = other DNA
ctgctcatggtatcaatcttatcga

SEQ ID NO: 2 - DNA, 19 bp, synthetic construct
  source 1..19: mol_type = other DNA
gataccacaagawsgccgt

SEQ ID NO: 3 - DNA, 177622 bp, African swine fever virus
  source 1..177622: mol_type = genomic DNA
actgctgtaggcgtcaaagattaaaattattactactgctgtaggcgttaaacattaaaattattactactgctgtaggcgttaaagattaaaaatattagtactgctgcaggcgttaaagattaaaaatattagtactgctgcaggcgttaaacattaaaattattgtactgctgtaggcgttaaacattaaaattattactactgctgtaggcgtcaaagattaaaattattactactgctgtaggcgttaaacattaaaattattactactgctgtaggcgttaaagattaaaaatattagtactgctgcaggcgttaaagattaaaattattactactgctgtaggcgtaaaagattaaaattattactgtaggcgttcatttcacaagatgcggaattatttcgcaaagattattttttgaaacgccgcggccggaaatatttttttttgcggttgtaattgatttttttttgcggctgggcggcgggccagacaaaattgaccataactggtgttacgccgccggtaataaaccctaccgtaaatacttttttttggggcggccagagagacattatcgccgtaggtatcaattactgctgtaggtatcaattattatactacaggcgttaaacattattagtacacaggcgtcaaagaagctaaaacttaatgttttttcgtcaaaaatcgccatgaatatctatcttgtgtggtttctctacatactattggggaacctgatattagcagtaatatattgcgtcatagatgaggtggtgtgtgacaatatccatataaaaaaaaatgttgccgcccctgagatgccgcggcggttctaaattttaatgtttttttcggcgaacatttttcacatatgcgatattggcgctaaagcgagcgtattaccgcttgtaacaacattttttttcgatcggcaatagataagtagaatataccatattattgctattgccatcaatgagaatgccacgtaggcataggtcatcctatggccggaccaatccatggctgcacttaaaaatatcaaaaaaagtttaagttttgggccggcgttaaaatttaaaccttttctggttgatctttagccatgtatagctgcgatgtttggtgccttatctacatgctattggcattcctgatattcgcactaaagtgctatgttacaaccgtcttatgcgtgatttttatccaccttattggccgaagggccgccttgtatttcctgttaggtggtttggccgtattctactggtggcaagcagctatcaataaaatttaatggctctcacttaagatccttgctgtaagcgggcgtttacatactttgatcaagaaaaaaaattatttttggacccccccccatgttttatacaaaaatcatataataaagtggcgacaatcaacatattaatcaaccacagcattttatgatgtgttaatcaacatataccatattaatcaaccacagcattttatgatgcgtcaatcaacatattattacggagagcgtcaatcaatataatattgagaacagcgacttgataccgtgtatggtggtggcggcggcatgttgtttgtaacagcatttttcatcattcgaagcttacaaaagatatgtataagatagcatattaatgttattaacagtaatatcaataaggcgtagctatagatcttcactttggtagaccaataatccatggttgcgcttaaaaataccaaaaaaaacattaagttttggagggtaagattggtttttcaccattggtaaagattattattctaaatgtttaccccatagatgtgaaacaatgattcttcatatattaacatattttttgacttatacttttcttcatctagtaaggcgttaattttttccggatctgtcgtttttattgataaaagagaagagtctggactgtaatttttaaataataagatatttattaatatccaattattcgtttggctcgctatttccatgctctcttcgaaagcatcagctcctaaatctatacaaaggaataagttaccttcacaaaaattcattaccgaggtaatcattgcccgattaatgtcagcccccaacataaaacaataatatatagttgtataattacaatcatacatacaggccaactgcatcatttcatcaatgtctatatttgtcttctctttgttataaatttcatgaaggtcaaagacgttgttataagcaaccccacatattaaccgccaatctttaaaatgactatatcgttgataaaaatattggatggcttcagtaagcttatatggtatcgccatactataccaatacctagttagcatttcgttgaatgaaatattatccaatgtaaagttaattgataatgtatctagttcaccaaaaattcttaatttcagttgagcattatttaggaaaaggggattatcagataataattcatggcatagaataatattactgctagttttaacatactgtacattataaaatatttctaaaattttattttcactcaaagctttcctcgcacctaacttttggcataggtcctggtgcactccatattgacagtaaccaacccaaagctgatgtctgcaccccattcggtaaacagctctattaaaccatgattgttttcctgtacagccttcattaatgcaacatttaatgttaaaccatgtttaaaacttgctgtttttattaatatttgttcatctatacaagtatgataaatcgtaattggggcttcatgccaccacaaaccacaacgctctaaaatacaataatcatcttttaacacaggctgtgtagctagtacttttttagtaagtgcttgtaaagtagatggcatcttctatctgcaaaataattatttccgaaaaaaaaatcaaattaaaatactaaattctatttttttttttaataaagcctgtaaattatataataaatctcgcccaccgtattatttccggacacaactttttatacctcattatatttttagatctatagttttttaacaaggcattaattttttctggatctgtcgtttttaaagataaaagagagacgtttgaactataataatctttaaatgataatatttctactaatatatcatgattcttttgttttgctaattctaagctctcttcgaaagcattagctcctaaatctatacaaaagaacaagttattcatataaaagttttttaccgaggtaaccattgcccgattgatgtcagcccccaatacaaaacaatagtaaatggttaaaaaattgctatctctcatacaggccagatatatcatttcatcaatattcatatcaaccttttttatatgatacatttcatgaagatcagacacgttattaaaagaaagcccacatattagccgccaatctttaaaatgactatatcgttgataaaaatattggatggcttcagtaagcttacatagtatcgctatactataccaatatctagttagcatttcgttgaatgttatttcattcaatataaagttgatcgatatcttctctagaaaacaacaaattattacttttaattcctctatattctggaaaaggggattattagataacaatttatggcataaaataatattactactagttttaatacgatgtattttataaaatatttgtacaatatccatttcattcaaaatttttgcgcctaactcccggcagaaattccaagtatgctccgtattgacagtgactaagctagagttgatgtctgcaccccattcagtaaacaactctattagatcatagttgttttcctgcacagttttcattaatgcgagatttaactctaaaccatctttaaaaattgctgattttatcatcaattgattatcctcattagtagaaagcataattggagctccatgccaccacaaaccacaatatttcaaaataaagtagtgttctttagatatgtgctgtgtggccagtatttttttagcaagagcctgcagagaaattggagtagacatatttttttttgcaaaatggtttaagtttttcaagaatacagattggataaattaggttgttgacttagttacaggaggtattaaatattatgtagacataaaaatgagatcctccaaaaaaataaacaacaaaaaaaaatatgtttaatattaaaatgacaatttctacattgcttattgctcttattatactacttattattattttagtagtgtttttatactataagaaacaacaaccaccgaaaaaggtctgtaaagtagataaagattgtggtagtggagagcattgtgttcgtggatcatgtagctcattgagctgcttagatgccgtaaaaatggacaaacgaaatattaagatagattctaagatttcctcatgcgaattcactcccaatttttaccgttttacggatactgctgctgatgagcagcaagaatttggaaaaacacggcatcctataaaaataactccatctccaagtgaatcccatagcccccaagaggtgtgtgaaaaatattgttcatggggaaccgatgactgtacaggttgggaatatgttggtgatgaaaaggagggaacatgttatgtatataataatccacatcacccggttcttaaatatggtaaggatcacatcatagccttacctagaaatcataaacatgcataaataaatacattaggctcatcgtatctttttaaaatccataaatattcgtttgatatatgctgaaatttttataaaaaaaaataactatttcctataaatcatctagaaatagtcctcgttttgatcggtttatatcttataatattgtgcatcgatgcacaactgctttttttggtccttctggaacatcattatattttctttcattaatataccattcagatgtaaacgttgaataatttttatggcaacaatctaccattgaattatatttagtaacatctaatacatcgtttgttttatcaggctcagctctataatcttgataatttttgttatcagcttctaaagctccatcattatttttcaaagaagtatccataattatgtttggtaaaaatactttaagttttaatgtgatatttaaaatggttgttatataaatttaccgcttacaggtaatctttattcagtgtcataaactatacttttgatgattcagtattttgtgaatcagtacatttattatcattaatatttttaggctgtttttccaatgttttattgttgcaatgagcctgctcctcctttgacgaggaagtgtctgttggagtcatctgtttaggaagagtatcatccatatctattatgaagaaaatatataaatattgatatacaatcaaaaatatttttgatcacgtctttgttatctatcgatattgttgataacgtcttgaataacctacatcatttttttacataaaaaaatagatataatttttattatatctcaattattttaaagataattatcaatacagcaaatatcataagctaacatatttttcgaataatagttttttagtaaagtattaatcttttcaggattggtttcttttgataataagataggattcgctttataaatttttaaagataatatattcacaatgatagaataaccgtatatatctgctaatgtcttactgtgttcaataacattagcccctaaatccatacaaaagaacatattttcaatacaaaagttttttaccgagattaacattgctcgattagcgttggctcccaatgcaaaacagtagtaaatggtcaaaaaattattatcgcgcatacaggccagctccatcattttattaatactcatatgaattttcgttgtgttacatatttcatgaaggtcaaacacattgttgaaagaaagtgcacaaattaatcgccattcatcaaaatgcctgtattcttgacaaaaatattgaatagcttctttaagattatattttaccgctatgccataccaatatttggttagcatctcactaaatgagatctcatttaacatagaatttgttgttaaatccttcaactcccaataaatgatcatccttaaatccaccatgtttacattttgtaaaaaagggttattagaaaataattcatgacacaaaatgacattactacttgttattttacactttgtttcaaagaaaaatcgtaaaatttcacttgtctcaagctcttctttagctcccaattttcggcataggtttcgagtatgctcgttattaataaaaagtaacccataattaatatttgcaccccattcagtaaacaacatgattagatcatcattgttttccttaactgccaataccaatgcagtattaagccttataccctctttaaagcataatgtccttatcattatttgattatcatcatctatatacattgagataggagcttcatgccaccataaaccataacgctctaaaatataataatcatctttagatacgtgttgcgtggccaatgcccttttagcaagtgcttgtaaagtcgatggctgcatgtttattctgttaaaaaaaaatcaaattatcgggtaaacataaggatcaacccgtagttaatatttgcagtagtattttttaacaatgaattataataaaaaaataattcattactatctattataaaacccatctttaactttaaagaagaactagatcatctttttttttgttgtgtcagaacttcttcaatttattacccacattttatctaaaaaaaataaaaactacatcatatcttgtttcttcatcaaattatcataccatttatagggtgtaggttgggaacattccatcatgtggtaatcagggtatttatatattttttgatagtaacatctatttggcagatgtattgtccaacaatcatgtctaataaaatcattttcacctatgggggaatcatcttaaaaaccttattcctacagattccattttgacagtcctagcaaaagtcacaatattttccatgagtacaccaatgttcaagctctctttcgggaggaatgctgccaattttatgttttttagcttctaactctctgtacaacatcagttgggaaagcagaaagaagattaccaggagaaccattaaatatataatagtctgcaaactacgtttgcgaatgtaatttgcaactaaaacacaacccacaaggtaaaatccataagttaataacttttgccattttcgtatgacagcctcgtgccattcatggttgtgttgtgggcattctgttcggtaaacttcatgaggctttatagaagttacatagtaggtacagaattcattgtgacgaaaaacactgcagttagctatgtagtcattttcaagaatgggagaatggttttcaaagaccttattcttacagatgccatcttgacagtcccaacagaacctacaatgatttgcataggtgcaccagtattcaagctccttttcaggaggggttcttgttagatccaggagctctagctcatatgtataaagaagagttggaatggatagtaaagtaaatatttgcagaccaagcatggctacttgtgaacaagtggctgctcgtcaacaaatagctgtttatcagcaaatagctgtttatcagcaacaactaattatcagcaaatgctgcttgtgggtaagccaataaataggccatacccttgaaaggagaattcagtttgataaaaaaaataacgagttttctaataacccggtcaagcatttaataaatgaatagcatcacacgtctgcatcgtgcattctgcctggaaaatgggcccatctctaatatatttacactgacggtgaatcatacagtgttccatgggatagctatgctcctgtacaggaggcatatcttttagaactttattcttacaaagaccatcttgacaagcccagcaaaaccgacaatttttcacatattgacaccagtatctaagctcctcttccaggggattgtcggtcgaaaacccctgtagactagctaggccagctagcagcaagccgaggtaactaaagaacctcattgtagtgttatattacgaaaaaacatgttaaaatttggaaaaaaaagccctttttatagatctggaaaaaaattttcacaaatctaattaaaagccttacagatcatccttttcataaattttcattaacaattggtgggggcggttgtgaggtactggatcagaacaatccataacatggtaatgtccatttccttcaccatatgtacactggttataccagcgagaaacctcacaagatgtcaaataactgttctcaacaatcaatggcatgctcttattcaccttgttcttgcaaattccatgtgcacattcccagcaaaacttgcagttttccatgtaagtacaccagtatccaagttcttcttgtggaggattatccgttgaacgaagatgccctcctgcctgagtaggtagtcctaagacctgattggccagcaggccaagaatttccaagaagatcaccaacattgctacggctggctgaacagctggcagatagctagctaattagcaaaccaagtgactcgccctctctactcttaatatgagaatttaagattcggtccggcttttttcccatgttttacagggaaaaggtatttttagcctatgaatgtacatggttccgcacattaaaaaaaaataaaagaaattatttaatattggctgttattttctttcaactagcaacaagccaggtaactaaagaacttcattgtagttttatattacggaaaaggttaaattttggacaaaaaaaatcatatctaattaaaaatcctcacagatctttcttttcataaattttcattaacaattggtaggggcggttgtgaggtactggatcagaacaatccataacatggtaatgcccatttccttcaccatatgtacactggttataccagcgagaaacctcacatgttgtcaagtagctgttttcaataatcaatggcatgctattattcaccttgttcttgcaaattccatgtgcacattcccagcaaaacttgcacctttccatgtaagtgcaccagtatccaagttcttcttgtggaggattatccgttgaacgaagatgccctcctgcctgagtaggtagtcctacgacctgattggccagcaggccaagaattcccaagaagactaccaacattgctacggctggctgaacagctggcagatagctagctaattagcaaaccaagtgactcaccctctctactcttaatatgagaatttaagatccggtccgacatttttccgatattttacaagaaaaagatatttttagctacaaatacacttcatatatccctaaaaaaaacaaaaatttatttaattttaactattattttctttccactctctctttaagattttgtaaggattccagggctttggttcagaacaggccattacatggtgaatcccctgtcctagatcatacatacatttatttagccagcgggaaactatacatgattgcacatactcattttcaagaattgttgtattctccaatttgccctcacaaaggccattttgacaattccagcaaaacttgcagttttctgtataagtgcaccagtattcaagttcttcttgtggaggattatccgttggatgaagttgtccagctggttgattaggtagccctaagacctggttgcaattcatggtatggtagatacccttatctaaatcatacatacatttatccagccaacgggaaaccagacatgatttcacatactcattcttgtaaattactgacccatctattttgtttatacaagtgccgtcttggcagtcccagcaaaattggcaactttccatgtaggcacaccagtattcgagttcttcctctggaggctcctctgttggacgaagttgtccaacgagctgacttgaaacctggctggccagaaggccaagaattcccaagaagatcaccaacattgctacggctggctgaacagctgactgaatagctagccaattagcaatccactgtacttttcataagatcatttaagattcggtcggcattttttcaatagtttgctaggaaaaaatttttaattttatagattcacactacttcattctcatgcttaggaaaaaaacaaactaaatcttacaatgtatctggatctaatgagaagctagaattcatcttttttcaaatcctttctgggatgttcattctttttccactccttccttgcaattttataaggattccagggctttgggtcagaacagttcatgctatggtaaatgtgctcctccacatcatatctacataggtcaccccagcgggaaacctcacaatattttacatagtcattctcaataatacttgtggagttgtttccccaaaccctgctggtacaaatcccatcttcacaatcccagcagaaccgacagctttccacataagtgcaccagtatccaagttcattctctgggggttcaaatgttagaggaagatgtccacctacccgagtagaagtggaggatgaaaccaggttgctactggccagcaggccaataattcccaggataatcaccagcattgtgctcaaccagcaacggctagcaacgactagcaactgactagcaatagctagaaatggctagcaatcagtagtagctaacgctctactctttataagaaaatttaaaattcgatcagatttttttagaattgagaatgagtaaaacgcttatattctttttctagctagaaaaaataagctagtttaagataggatttcccttactaacggtttaatttttagcaaaggtataggtaaaatacacttgtacttagctgcaaaaaaataagcttatggcgtataagccgccataagtttatttaattaaaatgttaaactctgtgataagactggaatcttaggcaggtttgatgtggagaacagcatgaaatacaagagtgcctgttacacgaataagttctctcaaaccggggatggtcatactcacatctatgaaatcctggtctaggagattcatttgatgcatgatggccgcacccacacttatgagacactgaagaactaaagggtttaattttgatctgaatggtactatataggatgatggcaatccatatcaagattagagcaatcaaaatcacctcctcaagaagcatgatgtagccttaaatcttagactgctttaaaccttaggccctcactatctttaatgaaggagtttaaattttgatccctttttcaagacccatttagaagaaaaaaataaagtttatatcaatctaattcataagtcatctctttcataaatcttcatgtattctctatgtggataagtatgggatgttggatttgcgcaatcaaaaactttaactctcgatgaatctcttcccccatttcctgtacatttagagcttccaacataggatttttatcaaatatttcatgacataaaataatgttattgctcgttttatgacgcattaaaccggtgaaaatttccttattatttaaactatctttagctcctaactttcgacacagctcctgagtttgttccgtcctagcacaggtcagcccataataaatgtttgctccccactcggtgaacagccttattacgtcatagttattttcttttatggccatgattaatgccacatcaagatgaagaagttcccccttaaagggggttgagcttaaaataacgtaattacagtagtgacataagctaatgggcttgttttgccaccataagccacaatattttaaaatataatgatactcctcaggcacgctctgtttggccacagcctttttggccagggtttgcaaggagagcatgataacttcttgaaaaaaaaactcaaattaagttcctacttttttaaaatattagtatggacagatctaccatcatatgaaggaattctttcatcgttaaacactgaagagataatactttcatcgtatagagaatatcatgtcaatccatatattgaatgttatatatcattaaacccatcattaatatagtgtttatgtgctatggacaggttttttgaatgataatcttttaacatacgttttataacttcgggatcagtttcttttaaagataaagaatcattcatgttataacaatttaatgataacatgctggcaatgaacgagttgtctttttgatgcgctagagtctttccctcctcaaaggcattggcgcctaagtctatacaaaagaatatgtttccgatattatagaactgaatagaatgaaacatggcctgattgatatcagcccctaagacgacgcaacagtaataaatcgttaaatagttatagttcttgcgacaggcccactttagcatttcattcatgtctatgcgaatcctctccttttcgtacacttcgtgaagttcaaacacattattgtaaaaaagggcgcacataagccgccaccgatgtagatgagcatatctctgataaaaatagcaaatcgcctccttaaggttacattctattgccatcgcgtaccaatatttagtaaacatctcgcttaatatatcggtttctaccattaatccctccagttgttcataaatcattccctttacttcaaaacgatttatggtatctaaaatgggattattagaaaatacctcatggcagaaaatgatgttactgctagttagatcacgtttcaatgtgtaaaaaaatcgtaaaatttcctggtcatttaactgttctttggcacctagctgcctgcacaggtctcgggtgtgctccgtgttgacagaaagcaaaccgtagttgatgtttgcaccccactcggtgaacaattctattagatcgtgattgttttcctccacagctttcaccaaggccgcgttaagatttgtgccgttcttaaaatacggcgtccatattttcttttgatgatacatgatagggccattatgccaccatagaccgcagcacttcaaaaaatgaggatggcatttggccggatactggctggccagcacctttttggtgagagtctgcagagagaggaccatatttcttttttttgaaaaaatcaaattaaaaaaatcatgcttgtttagcatacatgtaatattgttataattacgttataattacgttataattacgttataactatattataacaatggtataacaatggtataacaatgttataacaatgttataacgatgtatcattgatgtcatcattcaactaggccaacatactttttaatttatagttttttaatagatgatatattttgctaggatctgcttcttttaacgttaatagcgaggagtctgcactataaatgtctaatgataaatgatgagatatcaaatagtaattccgttgctctgctagggcctttgcctcttcaaaggcgtcggctcccagatctatacaaaagaacaagttatccatattataaaatcgtacgcaggcaagcatagctgaattaatattagctcctaagagaaaacaataatatatggttaaaaaattgttatcttttgtgcaggccatccgcatcatttcatccacgtccatgcggatcttttccttttcatacaaattatgtaggtcaaacagcttattaaaacaaagagcacagattaaccaccacgtatttagatacttaaaatgttggtaaacataagaaatggcctccctaagattatcctgcaatgccactataaaacagtatatcgttaacatatcaccatccgacatattacttaatatgtcggtgtcttctactaaccttttcaacttccaatatatggatgaccttatttcccttataatgacataggctggaaagggattatcattaaaaagtttaagacataagataatattactgctagtagtgccagggtgtattaatttaaagaacatgtgcataatcttctttttatccacgcggtacttggctcctaattcccagcaaaattctcgaacaggcggcgtattggcgcaaattaacccatagttgatgtctgcgccccattctgtaaacagttttattaactgatagttgctttcctttgtagccaacattagtgccgtattaaggtccaagccgtctgcaaagcttggcagctttatcagcatatgtttgcaatcaagggaaattggggccttataccaccatagtccgcagcgttctaagataacatggtactcaatagatacttgctgtctggctagtacctttttggcgaaggattgtaaggaaggaaacatcctgtttcttttttttttaaaaatcaattatctttgttcataatcaagaaaaatccccatatttattgagtgataattttttaacatgcaatttattttttcagggtccgtaacgatcgacaacagagaaataaccggattgtaatgctttaatgataaggcatgggctatcagataattttccttttgttctgccaaagctttgccctcctcaaaggcatcggcacccaggtctatacaaaagaacaggtttccaagattatagttttgtatggaaacaagcatggcttgattgatgttggctcccatgataaaacagtagtaaatggccgaatagctataatcttggatgcaggctatgtgcatcatttcatcaatatccatgcggaccctttctatttcgtacagctcgtgaaggtcgaacacgttgttgtaaaaaagggcgcacatgagccgccacctatgtagacgcgggtatttctggtaaaagtagcggatagcatctttgaggtcatagtccaccgctatcgcgtaccagtatttggttaaaacagtgctaaagctatcatcatggtccagcatgaaggttatctccatgagccctcttaactcccacatgatttcccccctcagatccagattatctataatccttaaattggggttattggaaaacacctcgtggcaaaagataatattgctactggttttatcgcgcgttgtatcaaagaaaatttttaaaatatactctctttctaaatattctttggctcccagctctttgcacagatcacgggtattttccgtgagagcacaaatcattccatagttaatatctgcaccccattcagtaaacagctttatcaagtcatgattattctccttcacggctttcatcagtcctatgtttaactcgataccttgactaaaacaggttgaccttataaataatttattgcgtcgaatatgaagcataatggggccattatgccaccacaggccacaacacttcaggacatgatattgatctaccggtatacactgcccggccagtactttcttcgtgagggattgcagggaaggcaacatgcctttccatcctttgacggaaatcaaattatctactaataactatcagtgtttatattaagtatttagatattatcccgggctggatacgtagtatcgctattcacatgtacttccaactctagccggagcctgcagggtcatttatttttaatattgattcttttttgtatttaatcatttagagaaggtcatcataggagccagatgttctctctccagaacttatgtcgaaaaacattacctaaccgtaaacttcctgaattttttgacgaatatatattacaactgctgggattatactgggaaaaccatggaactattcaacgagcaggaaacaactgtgtgcttatacagcaacataccctcattcccgtaaatgaagccctgagaacagcagcatctgaagaaaattatgagatcgtgagccttttattagcgtgggaggggaacctttactatgctattataggggctctagagggcaaccgccacgacttaattcgtaaatatgatgaccaaatcaaggaccatcatgaaattctgccattcattgacgatccagtcatatttcacaaatgccatatcatgcggcaatgcttttttgattgtattttatatcaagctgtaaaatatagtaagtttcgcgttcttctttactttaaacatagattagaggatgatttgcccttcactcatttacttattgaaaaggcatgtaaagatcataattatgaagttattaaatggatatatgaaaacctacatatctacaatatgatagatacctttgaatgtgctattgcccataaggatctacatctatattgtttggggtatagatttatatataacagaatcgtacccgataagtatcatcatttagatattcgcatgctttcaagcctacaactcctacataaggtggcagccaaaggatacttagattttatcctagaaaccttaaagtatgatcataataaagataatataaatattattctaacacaagctgcaacctataaccatagaaaaattttaatctatttcattcctcaatcaacccacgcacagatagaacaatgtttactagtggcgataaaagcaaaatcttccaggaaaaccttgaacttactactgtctcacctaaacctttccatcaacctcatcaaaaaaataagccattatgttgccacttacaattcaacaaatataataggcattctgagtatgcggcggaaaaagaagatatatttagatatcatattgacaaaatttgtaaaaaaagctatttttaataagtttgtcgttcgatgtatggatacattttctataaacccggaaagaatccttaaaatagccgcgcgaataaataggatgatgttagtgaaaaaaatatctgaacatgtttggaaaaatcatgcggttagacttaaataccttaaacatgcggtacacacgatgaagcataaagatgggaaaaatagactcatgaactttatctatgatcgctgttattaccatatgcaaggggaagaaatctttagcctcgcaagattttatgcaatccatcatgcaccaaagttgtttgacgttttttatgattgttgtatcctagatacgatacgattcaaaagccttcttttagattgttcacatatcataggtaaaaacgctcatgatgctaccaatatcaacatcgtgaacaagtatatcggcaacctgtttgttatgggagttcttagcaaaaaagaaatcttacaggactatccatccatttattctaaacaatacatgccttagtttattttttttgcggccgaaacattattcttaccctagaaaacgcttatagtcatcttaaatcataggtaaggaagatcatcatattttttgaaacgtaattttttaacgcatgatctatgatttcagggtccgtgcttttaggcaacggggtggtggccggactataaatctttagggataaaatgttctttataagctcatacccttcccctaaagctgtagtaccctcttcgaaaacatcagcccccagatctatacaaaagaacatgttttctatattatagtactgtattgagctaagcatggcttgattgatgttggcgcccaggacatagcagtagtacatggttgaaaggttgtggtctttgatgcaggcgatccgcatcatctcttctatgtccatatggatcttgtccttttcatacgcctcatgaaggtcaaacacattattaaaacaaagagcacatgttaaccgccacgtattcaggtgtgtatatttttggtaaaaatactgtatggcctctttcaggttatagcgtatggctatagcgtaccagtatttgagtagtaatgtactgagcgaaaactcattatttagcagatcggtttttactattaactcccttaactcccagaaaatttctatcctcatttttatattatttactttttgtaatatcggattgttggaaaacacctcatggcataaaataatgttactactagttttatgaaactttagatctataaaaatttgtaaaatttcttcttcattcaaggtttccttggcacctagctctcgacagaggtcccaggtgtgctccgtgttgacagataccagcccgtagttgatgtccgccccccactctgcaaacagttttataaggttgtagttgttttcccttacagccttcactaacgccgtatttaggtttaagccctctttaatacctgctgattttatgagccttaggttatgatcaaacgtgatcggagcatcatgccaccataggtcataacactttaaaagataatgttggttcgtgggcacgcattgtccagccaacacctttttggtcagagattgcagggaaggcaacatgtctcttcatcttttaaaaaaaaatcaaattaattagccgaataaatttttctttcgagggctttttaaaagagctctttaagagctctttaagagctttttaagagattaaaaaattattcttgctggcattctgccaagtatgcggcattcctatcatctatagtatattatgagaatattcccaaatgatggataagttttttgatttataatcttttaataaactgcttatttcttcggggtcctttaagtttagtggcaaggaagcatctgagctgtaaatatccaaagccaaactatggctcagaaaattataacctttttgttccgctatggcacgaccctcttcaaaggcattaccacccaaatctatacagaaaaatatattaccgatgttataatattgtactgaagtaagcatagcttggttgatgttgccccccagcgcgtaacagtaatatattgttaatggattgttatccttggtagaagccagacatatcatgtcatggacgtctatttggatgttttccttgtggtacatctcatgaagctcatatattttgttataatacaggagacattttaatcgccattcattaagatccgtatatttctcatctagaaaacaaatggcgtccttacaatcgtattgtactgctttggcgtaccaatacttcactagtaaaccatttaactcgtccgtttcttttatttctatgagcccccatagtcttttataaattaagccccttaattgtataacaaatttgttttctaaaataggattattcataaaaatttcatggcacaaaataatactgccgctggttttattgtgcattatcctggtaaaaatacggaaaatatcgttgtcctctagagtttctttggcgcctagctgtctacacaactctcggatgtgcttcgtattgatagaaagcaaaccatagttgatatttgcgccccactctgtaaagagctttatcagactatagttgttttccttaacagctattattaatgccacacgaaggtctatatcttctcctaaaaatcctgattttatttgtattcggccacgatccatacaaagcttgagaggagcatcatgccaccataggccacaatatttcaaaatgcagtgttcatctattgacaaacactggctggctatcgtctttttgacgagggtctgcagagagagcggcaacgacatgtttctttttcaccaaaaaaaaatcaaatgttctcgtctttaaaggttaattcatgttcttaaaatgttcatttcatgatagtgattaataatatggtttaataacgctagaaggcttgtttataagacagtcataagcagtctataagacagtctataagcagtctataagacagtctatgacttagtctataactataatttctggatgggctgtaagatactcttcggctcgtttcagattttttgaagtatatgtctttagcatatcatatatttcctggggttcggttacatctaataccaaggtcacatcacggctgaaaagctgctttactaagaaaatgttgctcaagttatacatataagctttgtgcgcaatgagttgtgccctatcaaaatcggcagcccccaaatcaatacagaaaaacatgtttaaagtattattgttatagatagaaagattcatgccataatcgagactagcccccaacctatgacagtaataaatggccgcgtaatttttttcccgcaagcaagcaaatttcatcatcagattagggctgatgcaaatctctttttcacgacacaactcgtgtatgtcaaaaatgttattaaaataaaggctacaagctacccgccaatagaggtgatttttatgccttttatagaaatagtgaatagcctttgtaaaattatgtcgtaatgccagggcaaaccaaaactttgttaataggtggtgcgccgtatcccccgtcaacggaatgtttgaacaggtgtacgtaactgtgtctaaagtggttctagttacggtttccaagagtggattatgacaaaacatgtcataacccagcagaactcctgcacaggattttagcctggccacttcttttaaaatttccagaagacggggttcggatacaggcgttaagcctcccagttccgcacacagccgctttagatacacggcaggaacacgtataagcccatattcaggatttgcgccccaatccacaaataaacgtataagttcaagattatcgctcttcacggcctttactagcgccgcttcgagacaaagatcatcctcagaaaaacactgtaaatgtttatacgaaaaaacttgcttacaattgttacataggtgaataggacctaaatcccaccacaaaccaaaacgctgcaacgtataatcatagtcacttgaaagataattgcatgccacaacttttttggccaacgtttgtaaagacaacatactaagtttaaaacatcttaaatctaagctagctaactttcaagaaaaccctctatccctaagaatatatcttataactagacttatagcagtaaaaatcaactttggttattctttttaatataaaacgtctaattacttgcaaaggactataaagcccattttcctcagctagaatttttattttttaatgaagtagggggatatgttttcccttcaagacctttgccgaaagcatctttttattcttcccgatgtttttggcgagcatgtactacaacgattaggactgtattggagatgtcacggctcccttcaacgcataggagacgaccacatactcatacgacgggatctcatcctttccaccaacgaggccttaagaatggcgggagaggaaggaaacaatgaagtagtaaagctcttgttactgtggaagggaaatcttcattacgccgtcataggagccttgcagggtgatcaatatgacctgatccataagtatgaaaaccaaatcggcgactttcattttatcttaccattgattcaagacgcgaatacgtttgaaaaatgccacgctttagaacgtttttgtggtgtttcatgtctgctaaaacatgctacaaaatacaacatgctccctattctccaaaaataccaagaagagctgtctatgagagcgtatcttcacgaaaccctatttgaactagcatgcctatggcagaggtatgatgtccttaaatggatagagcaaaccatacatgtttacgacctaaagattatgtttaatattgccatctccaagagggatctgactatgtactccttaggatatattttcctttttgatagagggaacaccgaagctacgttgctaacgcaacatctcaagaagacagcggccaaagggctcctccactttgtgctagaaacgttaaaatacggcggcaacatagataccgtcctgacccaagccgtaaagtacaatcatagaaaacttttagattattttctgcgtcaactacctcgtaaacatattgaaaaacttttgttgctggccgtgcaggaaaaggcttctaaaaaaacattgaacttactgttgtcacatttaaactactccgtgaaacgcatcaaaaaactaccgcgctatgtgatagagtacgagtccaccttggtgataaagattttattaaaaaaaagagtgaacctgatagatgccatgttggaaaagatggtaagatatttttctgcgacgaaagtgaggacgatcatggatgagctttcgattagtccggaaagagtcattaagatggctatacagaaaatgagaacggatatcgtaatccatacttcttatgtttgggaggatgatctagaacgtcttactcgtcttaaaaatatggtatacaccataaagtacgaacatgggaaaaaaatgttaattaaagtcatgcacggcatatacaaaaacttattatacggcgaaagggaaaaagtcatgttttatttagccaagctctatgttgctcaaaacgcggccacccaattcagagacatttgtaaggactgttacaaactggatgtggcacggtttaaaccgcggtttaagcaactaatattagactgtttagaaattattactaaaaaatcttgctatagtatcctggaaatcttagaaaaacatattatttccctgtttactatgaaagttatgactgaagaagaaaaaaacctatgtttagaaatattatataaagtaattcattataaaacaatacaatgttaaaattcaatagatatccatcattaatattgattatattttcgaatattatcttctatggtgcaagataatcatctagcgcgtgaaacatgtcctcttctcttcaggaactttgtcgaaaaaagctgcctgactgcatacttccagagttttttgacgactatgtattgcaactgttaggactgcactggcaagatcatggttcccttcagcgtatcgagaagaaccagatacttgttcaacaggaacccatccatatcaatgaagcactcaaagtagcagcatcggaagggaactatgaaatcgtagagctgttgttgtcatgggaggcagatccccgctacgccgtcgtaggagccctagaaagcaaatactatgacctggtttacaaatactatgaccaagttaaagactgccatgatatcttgccgctgattcaaaatccggaaacattcgaaagatgtcatgagttaaacagcacctgttcactgaaatgcttattcaagcatgctgtgataaatgacatgctgccgattcttcaaaaatatacagactatctggataggtgggagtattgcagccagatgctgttcgaactggcatgtagtaaaaaaaaatatgagatggttgtgtggatagagggagttctaggcgtcggcaaagttacatctcttttcaccattgcgattagcaacagagacctacagctgtattctctgggctactcaattatccttgagaatttgtactcctgtggacaggaccccaagtttttactaaatcatttcctgcgagacgtttcaataaaagggcttctaccctttgtaatcaaaaccatagaatatggtggaagcaaggagatagccataactctggctaaaaaatatcagcataaacatattttgaaatacttcgaaacctgggaaagctaggttcagtatggtgtactcactattgtagtgaatcgtatcctgtaaattttgtaaaaaagcttaaacttttgaccacatcatattgttttagaaatctcaaaccagtgaacaacagtcttatcatacattaaaattccagtaaaatttatattttttttggtaaacaaatgttttctcttcaagacatctgtcggaaacatctttttcaacttcctgacgcttttgatgaatatatattacaagcgctaggactatactgggaaaaacacggatctcttcaacgaataagaaaggacgctgtgtttgtacagcgaaacatcgtcctttctaccaatgaggccctgagaatcgcagcctcagagggaaacgaaagggtaataaaacttctgttatcatgggagggaaattttcattatgtgatcataggagctctagagggtgaccaatatgacctaattcataagtatgatagtcaaattaaagactaccacatgattttatcattgatccaaaatgcaaatacctttgaaaagtgtcatcagttatccaatagtaatatgtggtgtcttatacagaatgctataaaatataatatgctccctattctccaaaaacacagaaatattctgacacatgagggagagaatcaggaattgtttgagatggcatgtgaggaacagaaatatgacatagttttatggataggacaaaccctaatgttaaatgagccggagtttatttttgatatcgccttcgaacggatagatttttctttattaacaatgggttatagccttctttttgataacaagatgagtagtatagacattcatgatgaagaagatcttacttcattaccaacagaacacctcgaaaaagcagccactaagggatgtttcttctttatgctagaaactttaaaacatggtggaaatgtaaatatggcagtcttatctaaagctgttgagtataatcatagaaaaattttagaccattttattcggcggcaaaaatgtttatcacgtgaagagattgaaaacctattattaaccgccataaccaattgtgcatccataaaaacgttaaacttactcttgtcttacctaaactattccgtaaaaaatatcattggaaaaatagtacaacatgtcataaaagatggtgattataccatcatattacttttaaaaaaaaagaaaataaacctagtggaacctgttttaacaggttttatagattattactatagctattgttttataaaacattttatccaagagtttgctattcgtccggaaaaactgattaaaatggccgcgcgaaaaggtaaactaaatatgattatcgaattccttaacgaaaaatatgttcataaagatgatcttggaactatatttaaatatctcaaaaccctagtatgtaccatgaaacataaaaaaggaaaagagacattaattgttcttattcataaaatatatcaagatattcatctggagactaaagaaaaatttaaattattaagattttatgtcatgcatgatgcaactatccaatttctatctatgtgcaaagactgttttaatttagccggttttaaaccatttgttttagaatgtttggatattgctattaaaaaaaattaccctgatatgatacaatatatagaaattctatcgaaatctgagtaaaatttatttttttgatcagagtaagaaaatgttctccctccaggagatctgtcgaaagaacatctactttctacctgactggctcggtgagcatgtgattcagcgactaggtctgtactgggaaaaacatggttctcttcagcgaatcggagacaactatgtacttatacaacaggacctcatcatccccatcaatgaagccctaagaatggcaggggaggaggggaatgatgaggtggtacaactcctattactatgggagggaaacattcattatgccatcataggagctttggagagtgaccattatagcctaatacgtaagctctatgaccaaatcgaagactgtcacgacatccttcccttgattcaagacccaaaactctttgaaaaatgccatgaattagataaatcttgtaacattttatgtctcgtattacacgccgtaaaaaacgatatgctttgcattcttcaagagtataaaatgcatctaagtggagaggatattcaagtggtgtttgaaacagcatgccgttcacaaaaaaacgatattgtgtcatggatgggacaaaatattgcaatatacaaccccgaagttatttttgatattgcctttgataagatgaatgtgtccttattatctatagggtatacgcttcttttcaatcatcatataaataatacgaacgaaaatattaattctttattgacacaacatcttgaatgggctgccggcatgggccttcttcattttatgctggaaactttaaagtatggcggggatgtaacgataatagtcttgtctgaggccgtaaaatatgaccacagaaagattttagattattttctccgtcgaaaaaacttgtaccaagaagatcttgaagaactattattgttggcgatacgtgcagattgttctaaaaagaccttaaacttgttattatcttacttaaactattccataaacaatatccgtaaaaaaatattacaatgtgtaaaagaatatgaaacgaccgttattataaaaattttacggaaaagaaagataaatctgatagagcccattttggcagactttataggatatcatagctatacctatatggtagattttatgcgtgagttttccatccatccggaaaaaatgatcaaaatggctgcacgagaatcgagggaggacttgatcataaaattttccaaaaaagtttgcaaagagcctaaagatagacttcactatctcaaaagcttagtgtatactatgcgacataaagaaggcaaacaactgttaatttatacaatccataacttatacaaagcttgtcatctagagagtaaagaaatgtttaatttggcacgattttatgcacggcataatgcagtgatccagttcaaatcgatttgccacgatctctccaagctcaatattaatatcaaaaacttgttgttagaatgtttaggtattgctattaaaaaaaattactttcaacttatcaaaacaatagaaacggatatgcgttatgagtaacatttttagatgagggaagattctaccaaactaactaagacctttcgctagaatgtatcttattgttaatatagatgagatatgtcattgtgaaaaaatagattaggtaggttgtgaaaaacagattaaacttaaaattatgtgtattatgtaaaattttagaaataaaaatttatttttttttattgagggtacggaaaatgttctccctacaggacctctgtcggaagaacattttcttccttccaaatgattttagcaagcataccctacaatggctgggattatattggaaagagcatggatccgtccatcgagcagaaaaagacagcataatgatacagaatgaattggttctttctatcaatgatgctttacagcttgcaggagaggagggggacacagatgtagtacagctcttgttattatgggagggaaatctgcattatgccatcataggagccttgaagactgaaaaatataacctaatatgtgagtatcatagccaaattcaggactggcatattctcctacccatgattcaagatccagaaacattcgaaaaatgtcatgatttaagccttggatgtgactttatttgccttctccaacatgctgtaaaatacaacatgctttctattcttgtcaaatataaggaggatctactaaatgcaaggattaggcatcgtatccaatccctgtttgttttggcatgcgaaaatcggagaattgaaattattgattggataggccaaaatctgccaattcctgaacctgatgccatttttagcattgctgttgctacaagagatttagaactgttttccttagggtacaagattatttttgattacatgcaaagacagggaatcattcaattaaccaatggagttcgcatggttgtgctaaatcgtcacattagcatggcaatagataatggtcttttaccttttgttctggaaactttaaaacatggtgggaatatacatagagccttatcttatgcagtaacacacaatagaagaaaaattctggattatcttattcgccagaaaaatatagcccctaatacaattgaaagacttttatatctggccgtgaaaaatcaatcttccaggaaaactttgaacttgttgctatcttacataaattacaaggtgaaaaatgttaaaaagctggtagagcatgtagtaaatgagaaatccactcttgtgttaaaaattttattagaaaaaaaggaaaatctagtggatgctgttttaacaagacttgtaaaacattctacatatttccaggtgagagaatttatccaggagttttccatcagcccagaaaaattcattaaaatagctgtgcgggaaaagaaaaatgtgttaatcgaggctatttctgaagatatttgggaaaatcccacagaaagaattacttatctcaaacagatagtgcacaccataaaatatgaaagtggaaggcgatttttggtagacatcattcacagcatttaccaaagttactcactaaaacacgaagatattcttaaactggcaacattttatgtcaaacacaatgcaatcacccattttaaagacctctgcaaatatctttggctgaacagaggaacagaaagtaagaaactgtttttagagtgtttagaaattgctgatgagaaggagtttcctgatattaaaagtattgtgagtgaatatattaactacttgtttactgcaggagctattaccaaggaagaaatcatgcaagcctatgatgctttagagtagccatgtattaacattctgaaagtagaataaaatatactatatactaaaaaccaaattagccatttttaactatcttcttcttaaaaactctggataaaaatttatttttttttaatttgggtagggaaaatgttctcccttcaggacctctgtcggaagaacaccttcttccttccaagtgattttagcaagcataccctgcatttgctggggttatactggaaggggcatggatctatccaaaggataaagaatgatggtgtgcttatagagcatgatcttactctttccatcaatgaagccttaattcttgcaggagaagagggaaacaatgaagtagtaaagctcttgttactatgggaaggaaatcttcattatgccatcataggagctttgaggactgagaactataacctagtatgtgagtaccatagtcaaattcaggactggcatgttctcctccctttgattcaagatccagaaacattcgaaaaatgtcatgatttaagccttgaatgtgatctttcatgccttctccaacatgctgtaaaatataacatgctttcgattcttgttaaatataaagaggatctactaaatgtactatttaggcaacaaattcaaggactatttattttagcatgtgaaaatcggaagcttgagattcttacgtggatgggtcaaaatctgccaattcctgatcctgagcctatttttagcattgctgttgtcacaaaagatttagaaatgttttccttagggtacaagattgtttttgaatacatggaaaaccaaggacttcatttaacccaggtagttcgtatggttatgctaaatcatcactttggcatggtaataaataaaggacttttaccctttgtgctggaaattttaaattatggtgggaatgtaaatagagccttatcttatgctgtcacacaaaataaaagaaagattttagaccatgttgttcgccaaaagaatataccccataaaaccattgaaagaatgttgcatctggctgtaaaaaagcatgctcccaggaaaactctgaacttgttactatcttacataaattacaaggtgaaaaatgttaaaaagttgttagaacatgtagtgaaatacaactctactcttgtgataagactcttgttagaaaaaaagaaaaacctgctggatgctactttgacaagatatgtcaaagattctacatactttcaggtgaaagaatttatgcaagacttctccatcagcccagaaaaattcattaaaatagctgtgcgggaaaagagaaatgtgttgatcaagggtatttctgaagatatttgggaaaatcccgcggaaagaatcaggaatcttaagcagatagtgtgtaccataaaatatgaaagtggaagacaattcctgataaatatcattcacaccatttaccagagttattctttgaaacctgaagaaattcttaaattggcaacattttatgtcaaacacaatgcaaccacccattttaaagatctctgcaaatatctttggctgaacagaagaacagaaagtaagaaactgtttttagagtgcttggaaattgctgataagaaggagtttcctgatattaaaagtattgtgagtgaatacattaactatttgtttactgcaggagctattaccaaggaagaaatcatgcaagcctatgctttggagtatgccatgtattaaatttctgaatcagtaagcaatagatagattttagaatatgctgtattaagttagtttctgaataagtaattaatagatagattttagtttatgtaaaaatgttaacatttgttcataagttttagataccattttagagttacttttttagatattactattttagccattattatcttaaataatcactattttagataggtccccgtattaaaaaccaaattaaccattatctatgtttttaataatactttttaaaaaccctccataaaaatttatttttttttcataaaagtagagaaaatgttctccctacaggatctctgtcggaagaacctttttcttccacttgagcccttaggcaagcatgtggttcaacggctgggattatactgggaaggccatggttcagttaaacgagtgggtgattgctttatatgtgtagaccagatttggatgctatcaatccataaggctatacaaattgcagcctcggaaggaaatgagaacattgtcaagcttttcttactatggaaggggagtctacaatatgccatcataggagccttagagggcaggcaatatgatctgattcaaaaatattacaaccaaattggggactgccatcagattctaccactgattcaagatccagaaatttacgaaagatgtcatgaattaaatgttacatgtacctttcaatgcttatttcaacatgctataagagataacatgctgcccattttccaaaaatatggagaagatctgaatggaaacaggagaatggttcaacttctgtatgagatggcatgccgattacaaaattatgatatcatcaaatggataggatctaacctgcatgtttataacttggaagccatttttagcattgcttttgttagaaaggatttaactttgtattctttaggctacatgcttcttctgggtagaatgagtactgaagatagaaactttatctcaatcataacacgccatcttgaatacgcatcaaaaaagggactttttgactttgtactagaatctttgaaatatggaggtcaagtggatacagtgttgtttcaggctgtaaaatacaaccataggaaaattttggcccattttattcatgaaattccccgtgaaacggttgaaaagctgatactccatgctgtggagtcacgggcctccagaaaaacattcaacctgcttttatcttccataaactactgtgtgaacccttttgtcaaaaaactactgcacgctgtggtggaacacaagtacatgcttatcataaagcttttgctcgagcggcccaaaaagaagataaacctggtagatgctgctctattcaaacttgtaaaatactctacttatacagaaatagtaaaatacatgggtgagttttctgtggacccaaaaagggtggtcaaaatggcagcacgactcatgagagtggacctgattaaaaagatttctaatgatgcatgggaagataaactagagagaatcaagcaccttaaacagatggtaaataccatgaaccacagaaatggaaaaaatctattgatgtacaatattcacaatattactggatatacctatctgaacaccaaagaagcatttaacttaacaagattttatgctgtccacaatgcaacatgtttgtttaaagaaatgtgtaaaagctgttttgtacatgataaaatacagctcagagaattgcttgaagattgtttacatattgctaataggcatgattatatccagattgcagaaaccgcagatgaatgtatcaaatatatagatcttattacatttaagtaaaccatgtatatatcaagtaaatccagattaaatcaggctaattgtaaatagttgtagataccatataatgaatgttttattaggatagtagttcagttaagatagtagtttagttaagatagtagtttagttaagatagtagttatgttaagatagtagttctgttaagataatagtttagttaaaactagttcatgttaagttaatagttttgttaagacaatagttcatttaagtcaatagttcagttaagtcaatagttttgttaagtcaatagtttagttaagtcaatagtttagttaagtcaatagtttagttaagtcaatagttatattaagacattagttctgctaatacattagttttgttaagataataaaaatttatttttttttcatcagggtagagaaaatgttctccctacaggagctctgccggaagaacatttacattcttccttaccccttggctaagcatgtacttcaacaactagggctgtactggaagggacatggatctcttcaacgaatcggagatgaccatgtactcttacagcaggacctgatcttttccatcaacgaggccttaagaatggcaggagaggaaggaaacaatgaagtagtaaagctcttgttactatgggagggaaaccttcattatgccatcataggagctttagagggcgaccgatatgaccttatccataaatattatgatcaaattggggactgccacaagattcttcctttaatccaagacccgcaaatctttgaaaaatgccatgaattgagtaactcctgtaatattcgatgccttttagaacatgcagtaaaacacgacatgctttctattcttcaaaaacacaaggagcaaataagattacacatggcattaacccaaatactatttgaattggcgtgtcatgaacgtaaaaatgacatcattagatggatcggttattccctgcacatacaccatctagagactatttttgatgttgcattcgcccataaaaatttatccttatacgttttagggtatgaacttctcatgcacaaagtaaatacagaggctgcatatatagaattacccaatttgctatcatatcaccttcgaactgcggcggcaggaggtcttcttaactttatgttagaaacaataaagcatggtggatatctggataaaacggttttatccgcggctatcaggtacaagcataggaaaattgtggctcattttattcatcaggttccccgtaaaaccgttaaaaaactgttactctatgctgtgcaggctcgggcccccaaaaaaacactgaacctacttttatcttccttaaactactccgtgcacaccatcaccaaacaactcgtacacaatgtcgtcatctacagttccacgcttatcgtaaagcttttactcatgcggcgaaaaaacaagttaaacctagtagatgccgttttagccagacttgtaaaatattccacctatacagacattgtacaattcatgggtgagttttctgtgagcccagaaagggtgatcaaaatggctgcacgggaatccaggacctttctgattgaaatgatctccaaagctgcttggggaaatcacccacagacgttgattcatcatctcaaacaactaaccaataccatgaagcctcaatctggaaaagaccacatcatatataccatccactatatttatctaaactctaatatgctggtagcggaggaggaaaaaaatatttttaaattagcaaaattttatgcgaatcataatgcggtaaacaggtttaaacaaatttgtgaagactattatatattagatgcacgatttaaaacacttattttagaatgttttgaaattgccgtccagaaaaactatcctagaattgcaaatattgtggatgactatattcgattccttttttacaggggaaatataaccgaggaagaaattcgtgaagcctattctttaaaagatgctgaggtttatgtagatttaaaatggttacaacaaggagaaatggtttaaaccaaatccggtttaaactaaatccaatttaaactacatttggtttatcattagtcattgaaaccatcgaaaaaaaagctatttgtttatccccataaactcatcttttttttgtctcaaagtttgacactaaaattcagtgttttatagtgtttataattaagtgttttgcatgcattgcagaaattttcatcttttttaattggttcaataccacatgtcatacaatatgttgtttgattatcaagattaactttatgaaaggaaagtaagtgagccgcaaatttaaaagtaaaatatctttcatttaaaatgatcttatgaatgtattttcgataaggaggaatgaaagcatttgccaaaataaatcgcataaaaggcttggaaaaacccatatcttctaatcttttgtgggtataaaccctattttggtgttttacaaaaacttcattgttataatagtcgttatagctatcaatcatttttttaagtcctataatgcccaaggttgcacgcataaagccacagtttctgctccaaaaagcatgcacctgtaaagggtgcttttcatataaccaattacaaaatttcattccgcaacagtagcatgttatttcagtgggggatgtatagaataatccggcattcgaaaatttttcataattttttatgtcatggattgcgaagctttgatttcgtgcatctatggagctatagcctacatatttaggttttacttcaaataatcgcaaagagatgtatggatctatcgtatttattttaggaaacatttcataattttaaattcttatatataatataaaaaaaattacaaacatttgtaatgatcatcctcaattgaaggctgagttgtaggctttatttttctaattatacgaagaaggtaggttctcataaagccttcaagatgactattgatgtttccaatacattttctcaatgagttcataaacccagacattttgctaatggcttggcaaagtgccaacaagttgtccacaaagtactggtagattgccactagctatagctagctatagtgagccaacctctctgtatgtattttatatatttcattttttaatagatttaatatttttataaaaaaatatttagttttttatacaagaatgtcgacaaaaaaaaagcccacaattaccaagcaagagctttactccttagtagcggcagatacccagttaaataaagcattgattgaaagaatctttacaagtcagcaaaaaataatacaaaatgctttaaagcacaatcaagaagttattataccacccggaatcaagttcaccgtcgttacggtgaaagctaaacctgctcgccagggccataatcccgccacaggagagcctattcaaattaaagctaaacctgaacataaagccgtaaagatacgagcattgaaacctgtccatgatatgttaaactaaactataaagtcatattcttctttatcgttattatcttcaatatatttttgccaatcgaaatcgaataaattcagatcctggacatttaaatacttatcatcgtacattttaatataatttaaacatgagttgttgtcaaaaacttttagcgtttttgttaaaattatcatatgaataatttccttattaagagttgccggaataatacaaaacctatttttaggtacatcatccatgataatagtaaaattagtaaaaattgtttcttgtttttcttttgtttcaaataaacgttgtaaggttaaaggtttctcgttcaatggtttctttgaagataaaaagaatgtataatctggtttaaaggtatttttggtttcaatcgtgattccatctgcttgagcatatactaaaccagaccaaatataacggtccactattacaatataatttagcttaagtagcactgcaatttctgcgataaattcactacgatgttttgtaaataatttatgtaattgttccgatgacatttctatggttttatttaacacctgcaatataagatcaccggtggtcgtgtctggattaggaaaatgtatacatatagcattataatccatgcattccaatgtttcttttaatttcattgcctgtgtgctttttcccacaccattgattccctcgatggcaatgagtattccacgcatgattaataaaaggaaaaaaagaattcagtttttaacatttcttacaaatctttttttatacaacattgtacaacactgcattagcggtatatgatgttatagcttcattaaatatttgcttttatataatctttaccaacctatatttggtagatcactgcagatggtcataaataggccataactaagataaaaattatttcagacgctactacggtagtattattaaaatcatgtgtggcaatgtatgacgtcttaatagataaaacatttaaggaaaacaaatttgaataaaaaaaataattgttatgatggcgttgttacacaaagaaaagcttatagagtgcatctatcatgagctagaaaatggcgggacaatattgcttctaacaaaaaatattgttgtgtcagaaatttcatacattggcaatacttataaatattttacctttaatgacaatcatgatctgataagcaaagaagatcttaaaggagcaacatccaaaaacattgctaaaatgatttataattggattataaaaaatcctcaaaataataagatttggagtggtgagccgcgtactcaaatttattttgaaaatgatttatatcatacaaattacaatcataaatgtataaaagatttttggaatgtttcaacttcagtcggtcctcatatctttaatgatcgtagcatttggtgtactaaatgcacatccttttacccatttaccaacattatgtcgcccaatatattccaataaattagatatctttgctattaaaatagttaaaaaccttataggataattaggtactttattacgataaattatgatattttataattagttactttattataattaatctctttattaatgaattatcataagataactaattatttttttccatatatcagataataaatctgatatgggctaaaagtatgtttcaaactatttacaatagaatttctgttaagaaaacatacataatttgaataaaatttttttaaatatcaccgaaacaatcaacatggtgttaatagagtttttaacaggtttcttctatttatatggaaagagactgttttccattagtaaagtcatggacatgatatgtctagactattataccattattcctgctcctctggcgatgatgttagcggcaagactaaaaaactatgacctcatgaaacgactgcacgaatgggaaatctctattgactacgctctacttgtagtagatgatgtgccgtctattgactattgcttaagtcttggcgctagatccccgactagagcacaaaaaagagaactgctgagggacaacacgtttaatcccgtgtataagtatcttatgaactgttccggcttcccaacaaagagagaaaaaaacattccttgtgatgttcaatgcgaaagactgcaaaaaaacattataaaagaactggtatttaactgctctgtactgcttgaaatggtactgcacacagaaagagaatatgcatacgccctacactgtgctgcaaaacataaccaattgcccatcctcatgtattgttggcaacaatccacagacgcggaatctattttgttgaaaacctgctgttctgataagaacatcaattgttttaactattgtattctatatggcggcgcccaaaatttggatgctgcaatggtggaagcggcaaagcacgatgcccggatgctgataaactactgtgtcatgcttggtggaagatccttaaacgaagcaaaagaaacggctgccatgtttggacacattgaatgcgcacaacactgttttaaactgcagtcttacgtcgtggacacatcgaatacagacgacactgattaaagcgacaatcttacgtcatgaacgactgtcttttgagtatctatacttacattatatttttttatgaaaaaaatataaaggttgtatacaaacctttgtatacaagaaatttggatcattaaacaataattaatttggacacaggaaacgatctagatcgatcaaaaagctattttttttgcacacagaacatttagataattgagagattactttccatacttgttaagcttttttacacacaggaactttggattctgttcaggaagtttttcatagacattatgtttacagccagtaataataattttgggctttttcttaaaccaccggtggaaaacatccagcttgtaaagagggaaatgcatgtagagaggttttggtagtcatggttaagagatttgactaactccatgtttcctgtaaagactgcccagtcccaagcagtaaaacctctatgatagtctttttgagtcggatctgctccaaattttatgagagaaagcatatttaaagaacggccccgtattgcggccttcatcacaggagtcatcccattaaaattcggtaaacaaattctggtcccattttttccgaaatagcccaacaccccttccaggattaaatgattttttttctcagctaaataatgtaaagcagagtttccatctttatccctcctatgagggttaattatttctccaggataagattcttgttcaaaaagaaattttaaaaagtctatacgtccgtagatgcatatccacatgaataccgaggatccatttttatcgcatctattgacaatccacggatctgttttaaaaaattcctcaaatagtgtaagattcccatttctaatatgttttttaatccatttaacaaacaagttttctatctccctttctggaaacatgtgttccattttgaatgtcgcccctactccactatatgattttactcctttaatttttaatgtccttttttttcggacttctttggataagctgtttattaccatctttaaatgccttatagcggggaggagccaggcccttttcccatatgtgcggtaattcttggtgtttatgcttgcctttggcataaccaggccagtatttttcgatatattcagggtttgtttttacgtattctttaaaggtccgataggcttcttgaatacaggtaggctcaccggtataatttccatgttcatcttcctttaaaaagccattaaccctgtcctttctccacttaagattgtgctttccaaaaatgcgatcaagatcttgcgcctgctggggtggaatcataaatccctttttaggtcgaagctttttattttttccatagcttcggccatcgcgttgcgaaacagtggttaggacgcctgatagtctttccatgggcgtcgcatctaatcctatccatccaccctgatgaatatcaatggcaacaagctctcctttattttgggcaagccaagtttccaagaatgccatgctttcttcccagggataaggcccgccaacaccacgggttgtccaatcttgcaaggactccaggtccgacacctggtaaggctctaaagaagacggttccttgtttttgtactgcaaataagatttaatgacccatttataccatgtgtcgaaccgcagcgtggcgcctccaaagtgaaagccgtcgttgattttaggatatctgcaacatatttcaaccgtacgtttgagttctgcaaaagcggccttccaaggaagtctttcgctgcgggtaagacggtctattttgccctgcgtgccatagcgtatggcatgtcgtgccaattgcaacaattctgacaccgatccgtgggccccgatccagtttatcggataggcaacctccgaagggtttaaaagatgctcgtaaaagcgtggatcttcagatgccaaggcgtctgcaaaggggataatgctagaaaacctgtctagacatacgttttctgtgtttacttctaaaggtagaaaaatggttgcgtgaggcttttgaacctgcttgttcagcggtctgcatatgctttgaataatgtctctaggactatgtcgcggcgctgcaaaaaataccgcgtttagttctggaacctctacgccctcttgaaagagtcgacagtttaataaaataacgggttcctttgaggaacaaaattctgtaaatgttttgaggataacctgtcgcggcagggttgagtgagctatcagggcatagaccccttggtctaccaacgccgcgtatagctccttggcctgtttaatatcacgggtaaataccagcattttaggagccggtatattggtttttaaataggctaaggccattataatttgctttactatgatctgtttcgtggtctcctctttggtactcggttggtgggccaatttaggcgcggctaccatctgcaattcaaaatcatttacatagccggcctctatgccttctcgcagatagtagcgaaaggcaacgccgccaaaaagttcacgatttttcatggaaagcggggtgtcgtacctgggcgttgccgttaaaaaaagtcggtgcccttttttaaagttgagcaacacgtgggtaaagggccgtgtctcccattcgccgcaaatccggtgacattcatcgctaataataagatcgaaatcatccaccagtagcgtggaggattggtaggtggcaatcacaagaagagaaggggcctcccgtatccgttttgcaataaagacaggattggtggtcatttctatattgtcgtgatttagcacaatgcgggtctggtcagaccccacaagcaaaacgttcttcaaagaaattccatactgatagagtttttccagagtctgccgtagtagggacaggcccggcaccaggtacaaaacttttccttgaagataattggagaggataagataggcgacgcgagttttgccgcatcggcaggccatctgcagaatggccctcccacttcgccgcagctcctgatagcccatattggccgcctccttctgataaagtcgatcctcgattgcagtccgtgtctcatctgtagaaaaaaataatacgtcatctgcgaaatgttcatcttccacaggagttatcaccaggtgtctcagtttctccttgcttatcagcggatcagagggcaaagatggctcaaccactatcgtggaatcattcatctcataggcgggagaatcacacaaagtatagcttatgtccagacagtttgcaacatcctcagccaattgttttattttttcgggtaaaagacatacgagttctttgtttttgacgcgaaaaaactgtgcacaatataacacccctgcttcaattttttgcgcatccttctttgtagatgtttccaatgtgaaacaatacttccattcatccgtaaaacaggttgtataagatccatcatgaagcctagcggccaagtttcctgtgtgcccaactttatgtaaggattgggcctccagccagggatgaaccgccacgtaaaatcctgcgcacatgctatatcaaattgcagtttcttaataactgtacacaggatctgaaaaacatgtgattacaaaatttagataagaaatatttaatattaaaaatcacagaatacatgtcactgtgtagagagaaagccaaaaactcctcttgaccgccgtgggaaatcatccagggtagtaggttgtgtttcataaagttgtatgccgtagtgatcaccgtggactccagatggttattggcatctttgcaatactttgccatcttggcagaaaagacgataaatccacaaattctaccccagttgataagatccttaaacagctcagtcacaaccccagtaaactgggttttaatttcttgaacactcgtaagagaaaaggtaattgtaacctgtttgttcaaacactcatcataataggttaaaattttttttatttgttgttgatatgggctaagctcatgctctgaaatatcattaatgtaatatttaatatatcccactagtatttcattaatgatattatgatatattaactcttctccctccatagcggcaccctatatttttttatttaggtttcaatgttatcacaattgcgatacaattgtgatacaattgtgacacaactgtgttgtatacaacaaatgttaggccacgtatagcaacctatatgttaagaaatatttttatcccaacattagttggaaacgagcagccgcaaagaagtcatttaaaataagccatttaaagatttagaatttatatgtatacaactgtacaatggaagcagttcttaccaaactcgaccaggaggaaaaaaaggctctccaaaattttcatcgttgtgcttgggaagaaactaaaaatattataaacgattttcttgaaatccctgaggaacgatgcacctataaattcaactcatacacaaaaaaaatggagcttttatttacccctgaattccacaccgcctggcatgaagttcctgagtgcagagagttcatattaaactttttgagactcatttcgggacatcgagtggtattaaaaggccctacatttgtttttacaaaagagatcaagaatctgggcattcctagtaccatcaatgttgactttcaggccaacattgaaaatatggatgatctacagaagggaaatctcatcggcaagatgaatatcaaagaaggctaaataaaacaactaacatcaaaaaacattaaaggctatgttgtggacgatgcctttgtctcaatagtttcgaggtcatccaataactcatgtaacgtaaaaaagttggtccatttttttgaaaacattaaaagacgttcgtcttcataaataaaaaagtcattcgaaggaaaaatgatatactcaataccatagtcttgtaatattttttttaggtctctcagggtccagggatttaccaggcttctacgcgaagtgagcatcataaaaatatctaatattttttgcgccataagccagcgcggattctcattggcccacaaatcaacaataattctcttatcaaccgtgagcattcctacttgattcgaagaaatgattagatgcccagcagtccaccccatgagtagataacgcagcgttgtagaaatgtcacatatggaaggcattcctccacaacatgaacccaaattaggatgcgtgtgaaacacaaacatagcaggcttgttggccaccctgctataaatatcagcaggcatcatagcctcgctgccaaaataaatgttctctcctgccctataggggcttggaatgatttccactatctcgggtacaccgtttatcatattaatgcggccgcaccattcacggtcatcgtccaaaaattttttgatggcaccccgaacattgtcccagttaagcaacagagtattcacaatctcattacgctccgcccagtattccttaaaacttcttttagacttgctgagctgttcccaggattcgaactcagtccaatgttttttttcttttggggaagacttcccttttgaaacattttttgcggctccaccatctacactatgattttccaaaataatctccttcatcgtttgagttatatgggcattgctaagcaccttagtggtaacctgtttacctatgtgatttagcagaaaaccaagtttgtccatttgtgtctcaaccatttattcttaacaaaacaaaaaaaaattaaaaatcatcgtcgtttaaaaagagtttgaaggcaaacgcatcatccttaacacagttctgatactgcgtaggtcttaactcgaaaaagttggttttttctacttcattaagaaagaatttagtcatctgaggaaaagggtttcccaccttataaatgcttttgcactgcatcatgaagcacaaattatctgtaaagtagcgtatatattgaaatagcatttcttttgaaaaaccgggaactcttcctcttgccttgtcaaaggcatagttaataaactcatccaccaactccacagcctccttcaaaattttgtgaatgatcttttcctcgggaatgttatacacgtaatttgagataagaaaacacgcaaaactacagtgcatcccttcatcacgtgagataaactcattatagcttacaagccccggcataatattctgttccttaagaaactggatcgccacaaagtggttttgaaataaaatgccttctacggcggcgaagcccaccagccgctcacctagagtgttcctgtcggggtccatccactgccgcacccactgcgccattttttttatgatagggtgtttttcaatgccgctaaagatgcgctgttgttccttctcatccgggatcagcgtttttacctgtattgagtaggcttcgctatgaacgcactcttgggcagcctgcattgtataaaagtataacacttcctttactttaatttcgcgcataaaattggttaaaaggttttcgataacaatttcgtcggcaacaacaaagaaggctaaaatttgtttataaaattcgcgctgtggctttggcatggcttcccaatcatcaatgtccttacacatgtccacctcctgcgccgtccacgtcaaactttctaattttttataccagttccaacattcggggtgctgaataggaaaaatagtgaaacgttgggaattttcaattagtaattcctccatatttgaaataaatattaacatcttcaaatttattggctgccatggagacgttttttattgagacgttggcatctgatgtgtatggaaaggcgttaaatgttgatttagatagactatcgcaggcgcaggttaaatatacccttcaagagcttatttcctactgcagcgctctaaccattttacattatgactattcaacccttgcggcgcgtctttcggtgtaccagctgcaccagtcaacggcctcctccttctcaaaggcggtgaggctgcaggccgcacaatcctgctcacgcctgtccccccagtttgtggacgtcgtttacaagtacaaagccatttttgacagctacattgactatagcagagattacaagctgtccctcctggggatagaaaccatgaaaaattcttatttgttaaaaaataaagatggggtcatcatggaacgcccgcaggatgcttatatgcgggttgccatcatgatctatgggatgggaagagtggtcaatatgaaaatgattctgctaacctatgacctgctttcccagcacgtcatcacacacgcgtcgcccaccatgttcaatgcaggcaccaaaaagccacaactctccagctgtttcctgctaaatgtaaatgataatttagaaaatttatatgatatggtcaaaacggccggcatcatttcaggcggcggcggtggaatagggctgtgcttgtcaggaatacgggcaaagaatagttttatttctggtagtggtcttaaaagtaacggcatacagaattatattgtgctgcaaaatgcttcacaatgctacgcgaaccagggaggcctacgtcccggagcctacgccgtctacttagagctgtggcaccaagacatctttacatttttacaaatgcctcgcctaaaaggacaaatggctgaacaacggcttaatgcccctaatctcaagtacggcctatgggtccccgacctattcatggaaatacttgaagaccaaatacacaacagaggcgacggcaaatggtacctcttttcgccggatcaggcccccaatctacataaggtctttgatttggaacggtcgcagcacgaaaacgcacaccgcgaatttaaaaagctttactatcagtatgttgctgaaaaaaggtacaccggcgtcacaacggccaaagagattatcaaagagtggttcaaaacagttgttcaagtagggaatccctatatcgggtttaaagatgccataaatcgtaaaagtaatctttcacatgtaggcactatcacgaactccaatctttgtattgaagtcacaatcccctgctgggagggtgataaggctgaacaaggtgtttgtaatctggccgcagtaaatctagccgcctttatacgtgaaaatggctacgactaccgtgggctcatagaagcatcaggcaatgtcacagaaaatttagataatattatagataatggctactaccccacagaagccacgcggagaagcaatatgcgtcaccgacctattggcatcggggtctttggcctagccgacgtgtttgcgtctttaaaaatgaaatttggttcacccgaggccattgccatggatgaggccatccatgcggccctatactacggggccatgcgacgatccatagaacttgcaaaagaaaaaggaagtcatcccagctttccggggtctgcggcctcaaagggtctactgcagcccgacctatgggttcgctgtggtgatttagtttcctcctgggaagaacgcgtggcacagacgacgcagggtgtgttgacgccgaaaaggtggtcgcagctacgcctggcggctatgcagggacttcgaaatggatatgtcacagctcttatgcccaccgcaacctcctcaaattctacaggaaaaaacgaatgttttgagccctttacatccaatctatatacacgtagaacgttaagcggggagtttattgttttaaataagtatttaatagacgatttaaaagaaattaatctttggacagaagccattcaacagcagctactaaatgcgggaggtagcattcagcacattttggatataccggccgagatccgcgatcggtataaaacctccagggaaatgaatcaaaaaattttaacaaaacacgcggccgcacgaaacccctttgtatcccaaagtatgtccttgaactattacttttatgaacctgaactaagccaggtacttacagtgctcgtcctaggctggaaaaaaggtttaactaccggttcctattactgtcattttagccctggagcgggtacccaaaaaaagattataagaaactctgagaaagcgtgtaatgcggactgcgaggcgtgtcttctgtaggtgtctcgcggtaaaagagcagcggggaccatatggtaaaccccaacaagaggataatgaataaaaaaagtaaacaggcatccattagttccatattaaatttttttttcttctatataatggaatattttgttgcggtagacaatgaaacctccttgggggtttttacttctatagagcaatgtgaagaaacgatgaaacaataccccggcctccattatgtcgtttttaagtatatgtgtccggcggatgcagaaaatacagatgttgtatatttaataccctcgttaaccttgcatacccccatgtttgtagaccactgtccaaatcgtaccaaacaagcacgacacgtattgaaaaaaataaacttagtgttcgaggaagagtctattgaaaattggaaggtttcagtaaatactgtgttcccccatgttcacaacagattatctgcgccgaaactttccatcgacgaggctaatgaagccgtagaaaagtttttgatacaagcaggacgactcatgtctctgtaaatgtctcttcctttatgggtgacgtctcttcctttgccgaggaagtctctgttatgggcaagaggtttgaaacaacgcaaggactctgcttaatctgctgtctcacaaagggaatcaaactacctgctttcgtatttttaatgtagtaattacccttgttgtgatgaattttaagaccatagcgtagtcccagtactttattaatgaattttaaaattgtttgagggtccgttttattgggctttttaagcttaaactcaaagctgatcgcgcttaaatcatactgaacaaattcatcaacgagtttcgtcattaattgttcattggtcaatatattagggtcctgaacgcatttaaagccgcacttagttaatagcataatagcgtacatatgagattgaaaactataattaaattgtagatcatgatgctctgcgtgttgcatggcccattgatgaaagtttaattcctgagtttgtaacatagtgagcgactcgtatactgtctttccgcggcttatttggacacggccagtatagttctgttttgtcataaaactattgtattgttcaacaaatttgggagtaattttatgaccgtgccatgcataaaattcgagtagtttatacttttcatacgcaaataggtcttgctggtctactgtgatgccttcctttaagttttgtttaatttgtaaagctttattggcatcaatggtttcagccgaggcaatgtttacatagtcctggtgtttaatttccattttaatgcttgtatattgtttgactgtctccagcttttcacccgtcagtataaacaccttagcgccggtgtcggcgatctggttaataaatcgggttataaagtgattttttgatagatgttgtatccgcattgtttcgagccatagatggtagtatggagttttataatatatcggcctacctgtttccttactatacgtgaaggaaagctggtgattgcttatggtctgaaaaagggtgtcacgtttttgtaacgtaaacatttcaatgtcttcgatggtttctggatagtaattttgtttcccctgtaagcagattttataacacttactttttaattcacgcacgcggcccaacatttggcaacatgtttctacgtcacacgacatattgttaaaaaagccgtataaaacatcaaatctcttatcttcgtatgaaacacccgctgaaatcgtgggcgtatagataaggatatcaacgagcccccaataatacgatacattattaaaatgggattcccgttcatgagcagtgcttttagaactataaaacccaatttttttttccggaaactttttttggataaatgattgcaacagccgggcctccattaatgaatttgtagggataacaatttttttgtcttctagcaaatcctttaaaaggttatttaaccaagtttctcgtgaagaggtaaaataatacgtgtcatgctgggcccttttatattgattccagtgaaagaagatagggacatccccgcgaaaacgctgtagaatattatacgttcgatttcctaggtttgcgtccaagcatataacataatttgccgtttcgagcatccacatgaaaatggcaaaagagggagcaaagtatttgtgcaggccgctattgaattgattaaaaatcgattctacctcatccaaaataagtaggtctacaggctcggctgtggaggttagccggaaaagtgattctacctgaatgatgactctttcgtagctgtccaaatctccagttacttcgctgtacaatgtgaaattcggtagccgggattgtatattttttgagaagatctgtcgaaacgtcacaaaccgtatggtttgttgttttgaaatagaattattgccgtagtatttttgcaaatagttgcgcagttggacggttttacctattttcatttgagcctttacaacaagcgtagggactcgttcatattctcgcatactactttcatcatagatgtgtttttgagtatcaggcagttcttcaaagagaatggactcatgaacctctatgctctttgtcatcacttggtccacatatgtttccacaaaattatttgtgccggaaaggctgcccatgagaaggctatgtttattgtcatggcgacagtgttgatacactttgtttcccgtgactcttaaaattagggtattgtccttatcatgcatacgcttacatatttcgcagtaacttggacttgtacgtttaaacaatactaaatttttatgaacacggaggaagcaatgatttttacatagtgttcctgcaaattttaatacctcttcaagttcactttgttggatagtatcgcaggaactcggtgttgtttcttttacatttgtgaagatacaaggtaaacacgtcgtttcaaagggggttgctataagggtatcactctttttcgtggttgtactggtctcaaacacctctgcaagctcctcattaaacattttaacacgcatgctaccttttttatgagaccctatgatgcgaaaattttgaatacttttgttgacctgggggtcaacaaaaggataaacgtgtttgggaagattttctaacactttggatgtaaagactttggcctcattattgtttaatactgagtatgtataaagtatgatatgaaaggagtatttaagttctcgctttttatttaatccgatagaatctgttagcaaaatttgttcacgcgttagattgatgttataaggtaaagaatatgtctcgtaaaatacatccatgatgacgttaattatcatgtcaaggatgtcatagacattgtcttcgacattatcattgtcatcaacattgtcatcagagtatgacttatttaccggaaagtcgatgtcaaattttaagcgctgaggcaaaaacccaaataccacttcgtggaaacacttctgctcaaagggctgagccgcctcccactcccaaaagtcatcacgacttgaaaaaactctaaaaagattattatattcatctcgcaccacgaagtgattctttaaggtttcgagagaatatttatcctctacggcttctccttgggagttacagcgaagaaacttgaatgtttcttgcattttgatatttaaaattaaatcaattatgatgcggccgctaatgcggcggttgacgcggccgcgccgctgacgcagccatcatacataaagcggcatggccgttttataacgactagtcggccgttatatgacgaactatataaaaatgaattcttttaattagagttaagtattgttgattgtataatccatcatggttgagccacgcgaacagttttttcaagatctgctttcagcagtggatcaacaaatggacactgtaaaaaatgacataaaagacattatgaaagaaaaaacgtcttttatggtatcattcgaaaactttatagaacgttacgataccatggaaaaaaatattcaagaccttcagaataagtacgaagaaatggcggccaaccttatgaccgtcatgacggatacaaaaattcagcttggagccattatcgcccaacttgagattctaatgataaatggcactccacttccggcaaaaaagacaacaattaaggaggctatgcccttaccttcatcaaacacgaataatgaacaaacgagtcctcccgcctcaggcaaaacaagtgaaacacctaaaaaaaatcccacgaatgcgatgttcttcacgcgtagcgaatgggcatcctcgaatacttttcgagaaaagtttttaacaccagaaattcaagccatattggatgagcagtttgcaaacaagaccgggatcgaaagattgcatgccgagggtctttacatgtggagaacccaattctctgacgaacagaagaaaatggtcaaagagatgatgaagaagtaatatttttggtaaaaatatttttatcaaaatttttttaccaaataataaaaaatattttttacttttttttcttcataatatacatagaatgcctacaaaagctggcacaaaaagtaccgcaaataaaaaaacaacgaagggctcctccaaatctggttcttccagaggccacaccggcaaaacccatgcttcttcgtccatgcattccgggatgctctataaagatatggtaaatattgctagatctagaggcattccgatttaccagaatggatcgcgtcttactaaaagtgaattggagaaaaaaattaaacggtcaaaatgaatataatcaggaaacttaagcctggaacaattagccttgtgctgggacccatgtttgccggcaaaactacgtttcttattcattgcatttacatgctcgaacgtttggaaaaaaaagtagtcttcataaaatctaccaaaaacacccgagacaaaactattaaaacacactccggtatacagctacgacccaaacaatgtaaaatcatagaaagcacacagttatctgacgtgggttctctcaccgatatccatgcagttgtcgtagatgaagcgcatttttttgacgatttaatcacatgccgcacttgggcagaggaagaaaaaattattattcttgcgggactcaatgcttccttcgagcagaaaatgtttccgcccatcgttcgtatttttccttactgcagctgggttaagtatattggccgcacctgtatgaaatgtaaccaacataatgcatgctttaatgtgcgtaagaacgcagacaagacgcttatccttgcgggaggaagtgaactgtacgtaacatgttgtaacaactgtctaaaaaatacatttattaagcagttgcaacctattaaatattaaaaatcttatacaataatggatcattatcttaaaaaattacaagatatttatacgaagctcgagggtcatccctttctttttagcccgtcgaaaaccaatgaaaaagagtttattactctgctaaaccaggccttggcctcaacgcagctttaccgcagcatacaacagctgtttttaacgatgtataagctagatcccattgggtttattaactatattaaaacgagtaaacaagagtatttatgcctgttaattaatcctaaactcgttactaagtttttaaaaataacgagctttaaaatttacattaatttcaggctgaaaactttttatataagtcctaataagtataataatttttacaccgctccctctgaagaaaagactaaccatcttctaaaagaagaaaaaacttgggcaaagattgttgaagaaggaggagaagaatcctaagtcgcttacatttttttttgctatttttatagaatgtacacgcatgttgatgttgtcggaatagctgaagcctcagcggccctctacgtgcaaaaagatagggatcgctacttagacgtgctaacaaccattgaaaactttatttaccaacacaaatgcatcataacaggggaaagcgcccacctactctttttaaaaaaaaatatttatctttacgaattttactccaacaatgtggcggagcacagcaaggctttggcgaccctgctttataaacttgatccggaatacctcactcgttacacagtactcattaccaaaattcccaaccattggtatgtgattaacgtagatcagcgagaatttgtgcgcctatatgccatcccggcagttaaacaacacttaccgattcccattttacccttctattgcaccagcgcactcacccagcaagaattgttttgtttaggacctgaactgcagttaatacaaatatattccaagctctgtaaccccaactttgtcgaggaatggcctacgttgctcgactacgaaaaaagcatgcggatgttatttttagaacagtttccgcaaagattggaaatgacgggcgggaagaaggaggagaaggaaaagcatgaaagtatcattaaaaaaataatactagaaatggtctctacccgtcagcgaatcgttgttgggggttacatacaaaaaaacctgtacaaccatgtactcaagaatagaaatcgtttacagcttattacgagcttaaatatttatgaagaaaaagatatcatccagcaattttgtgattcaaatggactgaagatcaaaatacgtatcaacaatccgctcttgcctacaaatccggaattacggcgtttgactatttattttaatcataataatgatgatgatcagtcatatctaatagtagatatgtacaacacgggaagctatgagctagtgcctacaaatcagataaacacgcttgatggcagctttttaataggaacacccttcgtgcaagcgcgatttttgttggtagagatctgggtgcttatgcttattgcgcagcaaactaaaaaggacaccaaaaaaataatacaattttttataaatcaatatgaaatgcttatgaatagtccttggcccagtatggaggccctttttccctcaagcagtaaaagatatttaggcaactatgtagaccctaacgcgctcataaagtgggcacaactcaaattaaaaagaataccgcctttttatcctggaaagccggatgaagaatcatgttaagccgattaaaaaatcatgttaagctggttgaaaaatcatgttaagctggttgaaaaactcttggtgaaagcacggatgtaatattaacattggccgctcgcatttcgtgttgaaatacgatggaagagcgacggctatctaccatgccgatatcggcctggacatcacagttcatgcacttgtagatgggatgactcgcgttatagatggcaggctcgccacagtttctacagatgtaggagatgcagccatccgagtcgtcgtgcgatttttctatgatggtttgcatggcgccctgcgccgtaagcacccaatgctccatttctcccagacgaagacctccgtgcgatcgtttgccgtccaacggctggcctgtgagggcatccgtgggcccatagcttgcaacggcgtatcggtcatccagcacaaatttttgcaggcgctggtgataggtcggtcctatgaagatggccgcatcaaagtactcgccggtctggccgttgaacattttttggcatccattgaagcgtagaccttcttgcgccagtctttctgaaagaagctgcacattaataggcaggaatgcggtgccgtctgttaccaccccctgtagggcatttgctagaccaaccgtggtttctatcatttgaccgttggtcattcgggagggatgtgagtgggggtttacaatgaggtcgggctgcaatccgtcctctgtgaagggcatgtctgaagtgggcagggccagcgccgcaatgcccttgttcccgctgcgagaactcattttgtcgcctatattgagatttctttcatagcgcaggcgcatgaggccaaagatctcgtcattaggcccatggggacgcatcacagcatccacgacggccggctcatcgaagccgtacatgacagaccggtcgatgtatttgttgagttcgtctttttcgccccgtattttggccacttttcctataatgatgtcgccctttttgaccaccgttcctacgggcacgaatccatctacaagcttttcgtaattagcaccaggcttaagatttttggtgattaaagggtcgggcttcccaaacgactctatatcgctttctaattctactttttcttctcggtagaaggtgccggcaaagccgcccctgtcaataaaggactgcgacacgatcacagagtcctcctgattgtagccgccgtagatcatataagccacaatggtattaagcccgttgggtatgacatagttatgtgctatggtctttacaagcggcatttcattgtaaaactggaagaagcggttcatgtcgacacgatatggccagctaaagcaataccagccccccgtttgccggccttggtttgtttcataggtaacacgcgcaggttgggtacagtttgcgtagggggacactagggcggcaaggcccaaaatagcttggggcacgtccacgtgtgtgaaacgacgcgttacatcatgtttatgtttgcgtagctcgatgatggagaaggcaacaagacagttttccgcctcctcgggggtaatgaactcacagatgccctgtgctacgagatcttcaagtgtaagcgttccggctaaaatgtcttttgccatttgaggcgtaaatcgcgtattttgaatgaaagggattttatgtttttcccagtctttatcgcctttttttctggcctctgcggccttgtagcaggcttgattgtatttttcaatattattatctacaatgagtagggggcgggtcagcctaccgacgtccaaccaaaattctacttcgtctaccatgctatcccagtagatggtggtatggggatgcacaaccttgccctcacggcgaagcattctataccgctgagcaagctcaaaggcattggtgcagcagccgatccattctccgttgataaatacgcgcgctaggccctttcgtacaatgtccttgttggaaacatcggctaactgttgaatggccggatctgatagaaggcgttgttttaacgaaagtacttctccggcggtgcagacattggcagtgatggctaactgtttagacatgcctactttttcaccagtatcggctgactgggctacgcagatgtatccaggataggatgcgtgcacgcgacgcatcatgtcagccctttctgtttgtttggatgcgttggtggtgttatgagtatttaccgtacgcaatgctgaaatggtatttaataaattttttctttccaaactttgagtagatactctgtttacaatggggcgctgtcgcaccatgatggttttatttcctgaaatgatagactgttccatactgcgattaagatcggaggcggtattttttgataaagcggcagaaaatgcctcgataatgtttcgctgagtaagctcctcaaaggctgtttgtttaagaagttctttgaacccattgatgatgggtgctatcacggaagtattaaaaatagccttaaaggccttggcgagtgagacccctgagccgtgcacccgcttggtgcggtagctatcacggtccgtgggtggaaacacattcataatgacaagaagtattttatgaataagcaggcctaaaaagcgcagctttcgtacacgtgtatctgcggtttggcccatgtgtggcagcaatattttgtctaaaatagtaagttgtctttcatttaagtattgtaccgcattttcatcgcttttgtaagcagatgggtttgagacaaatttggaaaccttctcggataaaaactggataattttttctcggttcagctcgtgttggaccggttgaaatatggggtctaaaacatgaatggatttttccagaatttctatcatgaaggtattcacaagggagttggattctagatcaaataccacttgctcaatgatgctgtcatcgcctgtcattccaaacatgcgaaagatgagataccaaggtatgcgaagttttgagaacttggtgctattgatttcaatggtaatggcgccggtggtcatgtagcgtataataatttgagagctattttcgaaggcacctcccggttgggagataaactcgccgcgaatgatttcattattcccttgttgcatggtatggtaatggatgtgaagcgtgttaaagcggatgttttctaagaggtctacgacccattccccgcctcgggctataaagtagccgccgggttcattagggtcttctcctatttctttttttgcggtttttgataggtgatgagtgtggcagcggttgctgccccgcatgatgggaaatgtagatacctgaaaaggaggaatacttgctcgttttacctcctgccgaccattgctgtagtgcgccgttaaaataacctcggcggctagattaaccgggcccgaataggaaaggccacacaggcgtgccttattgggtagtaaatttatcttgtttccctgtgaatagtttcgatgttgcgggcgttcaatgttcacatctgtaaagttaaattggatctgaactgattcccgaagcttatctatttcagtatggtcgcgttggtctttataagtaatatccacgttaaacatttgttttacaatttgcggaattccattgtccataagatcgtcgaagcttttgatgttataccctatcaatcctgtagagtttactgcagcggagataaagctcagcatatcagcctctgtaagctcctcattatccacggtttcaatggggccgtaggttatttgcggccgcaagggttccatgattatgaagtactacattaatattcagttattctttaaaataaatctttatttataaatcttatttataatataagaatgccttatgcaagagacatcacaaagtttattacggcaacggaaccagaggtgggtcttcccctgttggcgctgcagcgctccaaatccatcataggggttattcttcttgtaataagtttgttatttattttcattggcattattatattatcagtgagtagtggtcataccacagcagcctctatatttatcgtattgagtcttatcctaggtggcggtggtttttttcttatttataaagataattcttaacccacataaaatttgaaaaaatatagagtaagaaaatgtccaattactattattactatggcggggggagatatgattggttaaaaacagtagaacccactaattttttaaaaatcgggttgccttaccaggcacacccattacatcttcaacatcaggcaactactcccccatctatcttagaaaaatttaaacgagcagacattcttcttaatgaggtgaaggccgaaatggacccactcatgttacaaccagaaaccgaaaaaaaactattccagatattgagtagtattgatatgttcaaaggtctgcgaaaaaaagtagaattcacgtacaatgctcaaattgttacgaatgcttggcttaaaatgtatgagctgctaaataccatgaattttaataatacatctcaggcattttgcaattgtgagcttccaggagggtttataagtgcaattaaccattttaattatacaatgatgcattaccctacttttaactgggtagcttcctccctttaccccagttcggaaacagatgccctggaagatcactatggtctttatcagtgcaatccggataactggttgatgcaatctcctttactgaaaaaaaatatagattataataacggggacgtaaccatcgctagcaatgtaaaaaacctagcgcttagagccacacaaaggctgacgcccatccatctatatacggctgatgggggtattaatgtaagacatgactacaataaacaaaaagaattaaatcttaagcttcactttggtcaagcccttacgggtttgttgagtcttagcaaaggcggaaacatgatactcaaacactataccttaaatcatgcatttactctttctttaatatgtgtattttctcacttttttgaggaactatacattaccaaacctacctcctctcggcccacaaactctgaaacctatattgtgggtaaaaacagattacgcttatttacccccaaggaagaacaagtccttctaaaacggctagaattttttaatgatacgcccctcgtagacctaagtctttaccaaaatttacttgaaagcgtttactttgccgtagaaacaatacatctaaaacaacaaatagaatttctaaacttcggaatgaaatgttatcgacatttttataacaagattaaactacttaacgattatttagctccgaaaaaaaagatttttcaggataggtggcgtgtgcttaataagctttatgttcttgaaaaaaagcataaacttaagctttgtgcctcctagggatctgttgcttaatttaacagatgcaatcttaacagatgtaaactaaaaagtgtgttcatacaaggattgtatttatgaatatttattaacatataaggttgtgatgtaacactgtataacctatataactacactatgaagcacggcgtataataatttatattgaacacgatgttgactcatttatttgcaaacaaatatttgtttgcaagacgtttgcatgcatttactaatatgttgttgactagtttatttgcaaactagatgtttgattgcaaactagatgtttgcacgtatttatttgaactaatatacactccttgttttatttgttatatacacagcatacataagtgtatattgtttacacttatgtttataactcgacgtaataacattttacacgctttttttttgcaaatcttaataatattgtatgataaatcaaacaatgtcttatatatgtggtttattattttaggcgccgcaagatgtactccattctcattgcatgcttggtgttattactctgtctagttatatatgtcggtcatcgtgccgatcatgcacgaaaatatttagaaggaatgtggcatggagatccggtttttctaaaacagtcggggctacaatccttttatctctacatacaacctgaccatacatgtttttttagcattgtgaataaaaatggtgaaaagctgatggaaaccaaaataccttgtacgataacaaataaaatatatatgttttttaaacctatttttgaatttcatgttgtgatggaagacatacatagctacttccctaagcagtttaactttctgttagatagtacagaaggtaaacttattttagaaaacaatcacgttatttatgctgtattgtataaggataatttcgccaccgcactaggaaaaacggttgaaaaatatataacacaaaattaatcatgttttctaacaaaaagtacatcggtcttatcaataagaaggagggtttgaaaaaaaaaatagatgattatagtatattaataattggaatattaattggaactaacatcttaagccttattataaatataataggagagattaataaaccaatatgttaccaaaatgatgataagatattttattgccctaaagattgggttggatataataatgtttgttattattttggcaatgaagaaaaaaattataataatgcaagtaattattgtaagcaattaaatagtacgcttactaataataatactattttagtaaatcttactaaaacattaaatcttactaaaacatataatcacgaatctaattattgggttaattattctttaattaaaaatgagtcagtactattacgtgatagtggatattacaaaaaacaaaaacatgtaagtttattatatatttgtagtaaataatatttttaattacttaaaatttttatatataagtttttgatactatattataaaacatatgttcataaaatgataatacttatttttttaatattttctaacatagttttaagtattgattattgggttagttttaataaaacaataattttagatagtaatattactaatgataataatgatataaatggagtatcatggaatttttttaataattcttttaatacactagctacatgtggaaaagcaggtaacttttgtgaatgttctaattatagtacatcaatatataatataacaaataattgtagcttaactatttttcctcataatgatgtatttgatacaacatatcaagtagtatggaatcaaataattaattatacaataaaattattaacacctgctactcccccaaatatcacatataattgtactaattttttaataacatgtaaaaaaaataatggaacaaacactaatatatatttaaatataaatgatacttttgttaaatatactaatgaaagtatacttgaatataactggaataatagtaacattaacaattttacagctacatgtataattaataatacaattagtacatctaatgaaacaacacttataaattgtacttatttaacattgtcatctaactatttttatactttttttaaattatattatattccattaagcatcataattgggataacaataagtattcttcttatatccatcataacttttttatctttacgaaaaagaaaaaaacatgttgaagaaatagaaagtccaccacctgaatctaatgaagaagaacaatgtcagcatgatgacaccacttccatacatgaaccatctcccagagaaccattacttcctaagccttacagtcgttatcagtataatacacctatttactacatgcgtccctcaacacaaccactcaacccatttcccttacctaaaccgtgtcctccacccaaaccatgtccgccacccaaaccatgtcctccacctaaaccatgtccttcagctgaatcctattctccacccaaaccactacctagtatcccgctactacccaatatcccgccattatctacccaaaatatttcgcttattcacgtagatagaattatttaatatgtactatatattaattatttaacctttcaagctggtcttcatttaaatttaaaatccactaataaaatgtattttctagtagcagatcatcgagaacatcatgtgattccttttcttaaaaccgatttccatcacatgcatcaaaatcctatacaaaaaaatcaagctctcctagaaatcaaacagctttttactggagattatctcatctgcaaaagcccttctaccattctggcctgtattgaacgaaaaacctacaaagactttgcggcttctttgaaagatggacgttataaaaatcgccaaaaaatgctgtcgctgcgagaacaaaccaactgtcaactttatttttttgtagaaggcccggcatttcctaaccctcaaaaaaaaattaatcacgttgcctatgcaagcattattactgctatgacgcatcttatggttagagatcatatttttgtcattcaaacgaaaaatgaggcccacagttcccaaaagcttgtgcagcttttttatgccttttctaaggaaatggtgtgcgtcgttcccacctccctcacccccacggatgaagagctatgcatcaagctatggtcttctctttctggtatttcaggcgtgataggtaaaatcttggcaaacacttgttccgtagctcatttggttcatggaaagctttcatcgcagaatattgatcagttaaaaactccctccaaccgaccattccccaaaaaagtaaaacgtatgcttataagcattagcaaaggaaataaggagttagaaataaaattgctctcgggggttcccaatatcgggaaaaaattagctgccgaaattttaaaagatcatgcgcttcttttttttctaaatcagcccgtagaatgcttggcaaatatacaaatcgttcaaaaaacccgtacgattaagttgggaatgaagcgagccgaagcgattcattattttttaaactggtgtggctctgcccatgtaaccgatgatagccaaaatatcacagaggcgtcgcggtccacaatgcaggtcgcgacgcagtccgccgcaatacagcccgctgcaacgcagccattgcacgaagtatcagatgatgcatcatcagatgcttcatcacccgtagggtatcaaacattatctaaagaaatgttattgaacacagcctgatgttaataattcactacatctaaagaaatgttaacctcgatactaaaaagtcattgaacacaactactggggcgctaagttgtccaacacatctaaagaaatgtcaacatcctcgatgctaaaagggtcatcgagccggtcaataatgtcttccccaaaaagtccgggagaactgtaggccgagatgtcgtccatggagctatcttccccagagcacacaaagtcctctccaaaaatcataaagttaaatgcaccgggcttacttaacagcttttcgctttgaataatagtgttgagttctgtcagcgcaaactctctcacaatattcacaacccaggagggctctttaatttcatacagcgttaagaaacttatacataaaaattctatagagtaaagcaaggcgctggcaggatctgttacccgtaggtgtttaaatgtagtgtgatattcattcacaacgttaggcagcaccttttccaaatcctccttttcctcgtacgacaggtgctttacaagcctttcaacatgtataggaggcttgttaaatgtactaacgtgccgcaaacagttataattatataagaaaatacgtacggcagagtcgaccgccatgagccttggatcatccattgaggtaggtggtggcggggcaccctggccttccctgatgtctgcgtaggagcgcccctccatggcccctatggcctctatcacagcaggactgatatccaaaatcttggccgtcttgattatttttccgtaatcgaaagtccatggctcctgtggaggcttgggttgtgtttcggtggagggcgtggtcatatctttctttatttgaatagaacggatcgacatcttttccttatcgtactggtctttataattattataatagtcatgaactaattcgggttgagaaagatgatcgtatataatataggtaaaaagtccgcacttgacacattttttatcctggaagtcgtgtaatcctcccttggggcagcgtgactcgtagaaggcataaaaggtgttaaattctaagctcgcctttagggctgtttggaccttttttatgtttaattgccccacctcatgttgtagcacgtggcatacagaacagcgtagatcggcaagtgcataatggttgtcaattttttttatgacgtctttgcgtgttacttcaatctcggcgggtttctgcgaactgtctacggccttgtaaacgtaaatggtccacttatgaggaagccccctttcatcgtatagggttgaaatgggaagccttttatactcaaacagccgagtccgttggtcggctcttcctgtgttaggatcaaatatgttataaaatccttgctgagcaagcagggccttttgctcgccataagcattttcgtacgttttgaattctgcaagttcggagttaaaattaggtgcattttgtaaatacttaagaaataattcataggctctaaggtaaatgagagttgaggttttttcctcatcccgtcctccccaccacacccgcaggctttcttcttgaaaatagatgtcattcagacgcgtcaactgcgtaaaatcaggccgatatttagaggtataaattttatcataaaattctttttgcgataatagctcggccggggtacgtcctatcacggttttaaactcatattcagcctccttgggagtccgtggtttgtgcatagggatgctgccgtcaatacgggccactgtggcagcataatcatacatggggtccagcagaatctctgtcaaaagtaccttggtgtcgtcctgcacgctaagcccttgtagcccattttggtggataatttttttgaaagcctcccgaaaattattagcaatccactgatccgtaatctcagatagctgatttattataccgctatattgctgcatcattttctccaaaagaaaggtcacgtatgcattcaaagagctatccgccttcattccatgaatggtaatcgtaagaaattctttatttttttgcgagctataaatgagattcaaaatataggcatagatgtagatcacagcatacagctgcgttaaaggatcgtaatcctcttcctttttaatattttcgatgctatacacgagcggcaggcagacatttacggctatattggcaaactgtttcacgtctacaagctttccaaagtggataaacgtgcaggccttcatggtttcctgccaaataaaaacacggagcttactattaagatcgccgatgatgcccacatctgccgtacgatcctcttgaataaaatgggccagctcttcgccacaaattttgcaaaagtaggagtaaataagcccctggttgttttctttctccttgtttattcctgaaaatttcattagcttggttcgcatggtgtcgtaggacgcttctgccgcttgaagctgtataagcatgtccacatggggacaaagcagcttaaacccgcaggctttgcatagattccaattggtggtattgtttttttccttgtagagtacacgaatactttctaatacttttaataactccgcgtattgaagacccgaacgcaactgttttaccagcttgagatgagcacatgcatttttttcttggagttcccactgttttttaatgtttaggtattctgttgtaataagttctgcctcctgtttcccacaggctttaatgacttcttgaaggatgctgttagggtcatccactttaccctccattgtaagaatttcacgtatagcatccgactgcaccctacctattttttcttccataattttaaaatactgtctcgcctgggtaatgacctctgtgagcttcatgtccacctgctgcagaatcatttgctccttttcacgctgttcagcatgttgtaaaaacttttgttctacagggttccaaagcacctccaaatagcctgctctatataggtcataaagcaagggcatgtatcccgatgtaaaaaccggggacaccgagtacatcgtagacaactcttttaaaaaaaatatcacgcgcttaatgttctcctccggttcaatctcctcggtttcaacgatattagatatatgactgccctgatcctcacggtctagctttcggtgtaccatctcctctgctagccgattaatgagccagctatgcccgccgctccgcaaaaacttataaagttcgatatactggtgcgtaaactggatgatgttttccttggtggttacgacaaccccttctccgttttttttccaggtttcttgatccacgcatttcataaatactcgaataaaattggtcaaattggctcctgaggcgacgtagcccaaggtttcaggcgagaaggagcctatctcagccatacgcataaaacactgcggggaaaaagtttttagccgcaacttaagtccatagatttcaatgggggcttctgcgggaacggccaggtgcgtcccattaattaaaaaaatttctttgcgtgtgctagggcgaacacgtaattccttttttttttcactcacgatggggaccacatcggggtctaccagcagttgacgtatgtaggcctctatgggcatggatagatcgggcagctttgactgctcggcgcgaacatggttcacaaaatcttttagagtgaaaagaaagtctattaaacgtatgttttttatatcattagaccctttaagggtagagtagatttcatccactagtgcctcgatttcctcattattgagcgataagatatctgtgccacggtggactatttgcgcgatcgtaattacttcctccattagatagaaactgaatattatatttaaaataaatacaaaatgtcaaatgaaagttttcccgaaacgttggaaaacttactttcaatgttacagaccaaacagcaaaacgcaattcagtcagaggtgattgaatggctgcacagcttttgtgaaacctttcacttaaaaatacactgccataaacagtttattcctagcggggaaaaaaaacgagctaaaatacccgctcaagaaacacagggaaacacgcagccctcccaccatgtgtaccgggttgttctctccagagcacagccagtcaaagcacaggaatctctgctaacaaccatgtgcaacggactggtgctagatgcaaacacatggacatgcctagccattcctccgcctgcgccctttcaacaggcgacccgccaggtccaacacttttaccgtaacaatttctacgaagtggttcccatccaggatggcacccttctcacaatctaccactgggatgaccctgaatatggcccctcctggtgcctagcaagtacccacggatatgatgtgagtaactactgttggataggcgacaaaaccttcgccgagcttgtatacgaattgctgcagcagcactctacctgcgacgtcaccctggaaaaaaataaaacgcggggaacgcgtcttttctttgataacttaaatcccgattactgctatacgattggaatccggcaccataatttacagccgctcatctatgaccctcaaaatatttgggcgattcaatctacaaacctaaaaacgcttaaaacggtatatccagaatactacggctatataggcattccaggaattcagagtcaagttcctgagcttccccagtatgatttaccttatctaatacgatcttataaaactgctatgaatcaagccaaaaatgctataaaaaatggcaaaaaagacaagggatactttaattatggctatttactcatttcgcgagcgcctgccattactaaaagtacttctaatgttttgttaaaatcgcctctgctggtatttttacaaaaaagtgtgtaccagaaaaaacacaatatctctaacagccagcgactagaatttattatactgcaaaactacttgatgcagcattttcgagatcatttcattgctctatttccgcagtacatatcctattatacgaaataccaaaacatgttgaatatgattatccatagtattgcaactaaagataaagatcatccctttgcaggagccgtggtaaaaaaagtgttggaagatattgaaaacgccgaaaacattattgatcatacaaccattcaaaactatgcccatcaaagcaagtacgccatgctttacttgtcaattatttcccatttttaatctaatacggccaaagccgcgggttttttaataaactaacatttaaaaaaactgttttattaaaaattataatacttttattatatatggaacatccatctacaaactatactcccgaacagcaacacgaaaaattaaaacattatgttttaatccctaaacacctttggtcttatattaaatacggaacgcatgtccggtactacaccacacaaaatgttttccgagtcggtggctttgtgcttcaaaatccctacgaagccgttataaaaaatgaggtaaaaacagcaataagactgcaaaatagttttaacacaaaagcgaaagggcatgtaacgtgggccgtcccatatgataatattagcaagctatatgccaaaccagatgcaattatgcttaccatacaagaaaatgttgaaaaagctcttcatgctttaaaccaaaacgtactgacgctcgcatcaaaaatacgttaaatataatttttgtagaggataaaaagctattttagctaaaaaataattcatatacgtttatgcagaggaagaacggtggctttcaaattcagattgcatccacgtagaccgtagcgttttttttgcttctggtttatatcgtaaaccgtaataaacatcatcatttgtatccgttggatctttttcccactccggataaaaaatcggttttctttttttttggtcgttttttgcagtaagctgtaaattaagggaatatagcttatcgaaaagttgttcctgatccatataaatagcagcatatattaaaaaaaaataaaaaaagacgcttcaacgagtcagtaccactgcttgccaacgatttacgttggttggtgcattatggtgatatagtaatgagtgcctgcacaagtgcttgcacaagtgcctgcacaagtgcttgcacaagtgcttgcacaagtgcttacacaagtgcttgcacaagtgcctgtacacattactgcatcgccaaagcacctgcaatgcctacttcctcaacagagtacgataactaaatgcttttaagcaccgcttgcgtcgatgtgtccttcggggcaatcgggttcaattggatccaatattattagtcataattacctaatacttattcaattttatcttttttaccttgtaagatttaaacagcgttttagcttgtttaaagcaacgtttaaaacaagctaaaatgctgtttaaaacaacgttttaaacaagttaaaacaaataagcttataaatataccatgacaaaattagcccaatggatgtttgagcagtatgtcaaagatttaaacctaaaaaatcgagggtccccctcgttccgcaaatggctcacattgcaaccctcactgctgcgctattcgggtgtgatgcgtgctaacgcctttgacatcctaaaatatggctatcctatgcagcagtcaggttatacggttgctacgcttgaaatccactttaaaaatattaggtcttcctttgccaacatttactggaaccgtgatagcgaggagcctgagtacgtctgctgttgtgccacctatcaatcgcacgatggcgaataccggtatcgatttgtttggtaccaacccttcatagaggcttataatgccatagaggcggccctggatcccctggaaaccattatcctgaacctcattgcggcacgagatctagacttcgttgttcacatatttccttataataagggccatgaagactatttggcctccacgcaacttattctcaaaatctttattgcgacgcttttaatggacattttaagaattaaagacaacacgttggacgttcacttaaattccgactatattattgtgatggagcggctttggcctcacataaaggatgccatagaacacttttttgaagcccataaggacttactagggtacttaattgcctttcgcaatggggggaactttgcaggaagtcttagaccctcctgtgggcaaaagattgttcccctaacgattcgagaggtcctacaaatgaatgatattaatttagccgtatggcgggaggtgtttattatgcaggaatgttccgacttagtcatcaatgggatagcgccctgtttccccatttttaacacgtggacgtatttgcaaggtattaaccagattttttttgaaaacacgtctttgcaggagaaatttaaaaaagattttattgcccgagagctttccaaagaaattatcaagggccaaaaaacgttgaatgacaaggagtttaaaaagttaagcctacatcaaatccagtacatggaatcctttctacttatgtcggatgttgccattatgattaccacagagtatgttggctatacccttcaatccctgccgggtattatttcgcgatccagctatttatcccccatcgtgaaaaacattttgatggacgaagactcttttatgtccctactatttgacctatgctatggcgcctacgtgttgcataaaaaagaaaatgtgattcacgcggatttgcacctgaataacatgacctactaccatttcaacccaaccagttttacagatcgcaacaaaccaggaaaatacaccttaaaggtcaagaatcctgtgattgcctttataaccgggcccaaagtcgaaaccgaaacgtacgtgttcaagcacatagatgggttcggctgcatcattgactttagcagagccattatggggccaaaccatgcaatcaagcttgagcggcagtacggcctcgcttttgtaaacaccttttaccgcaatcaaagtgagcatattttaaaggtattacggtactattttcctgaaatgctaaccaatcgcgaaaacgaaatacagggggtgattttatcaaactttaatttctttttcaatagcattactgccattgatttttacgccattgctagaaacctacgtagtatgctttctttggactatttacacacctctgaggtgaaacgaaacgtagaaatttcgcaaacatttttggatacatgtcaatttttggaggaaaaggccgtggaatttttgtttaaaaatcttcatactgtcttatctggcaagccggtcgaaaaaacggccggggatgtgcttttacccatcgtatttaaaaaatttttatacccaaatattcctaaaaatatattacggtcttttaccgtaatagatgtatacaattataataatataaagcgttattctgggaaagctatacaaacgtttccaccctgggctcaaaccaaagaaatcttgacgcacgccgagggtcgtacatttgaagatatttttcctagaggagaattagtttttaaaaaggcttacgcagaaaacaaccatttggacaaaattttacagcgtattcgtgagcagcttgctaatgaaaatttgtaaggcttgcagttcttgtatggtcagaacctatgtcgatggaaacattatttttcgctgcagctgcggcgaaagcgttcaaggggatagtcagaacttgctcgtctctagcaaggtgtaccacaccggggaaatggaagataagtacaagatttttattaaaaatgcaccctttgaccccacgaattgccaaataaaaaaggattgcccaaattgtcatttagactatttgacacaaatctgtattggaagccaaaaaatcattatattggtgtgccgctgtggctatatgagcaacagaggataaaccatatcatcccaccgaattatgacattcctttaaaaccgtccgcctaaatagttttcacacctttggtggcagactattttataaaaagtaatgttggttcatgaagataaagtgtgccaaagaaacttttataaacaaatgattaatgtaggtgctagtcgtgtgtacttaaacagggtattctatagccaagtattttctatagccaagtattttctatagccagtattagtcaagtatttagatgtcagggtatttttatagccagtatttttctatatgtacaaactattccagtaaacatatgtgtgttctttattgagcagcatcatggcattaacaagtttattaaactgctctaatgggcattaaatgacaactcggtgcttagcaaaagtgcctataccttttaacaattagggccgggaggcattcccagcttttttctataatcagccatacagtacccctgagcctcatacacgggaataaggtccttccattccttgttgggatcggcgggccagctctcaaatgaggtgtgaatgtaagggtcctgttctttttccttaatgaagcgtttaatctccatttgatgttgtttacttttttgtttgcggcggagcgtgttccgcaccaatacgtaaaaaataccaagaatcacacataaaagaattattaaaaaaaatatcatcatcgcggggtttaaaaaacgatcccatgcaacaggaatcgttcttaaaaccttgtctggcagggctgtaaacatgaagtctcctcctataatcggggtgggactgtagcctaacagttcaaggtcctgtcgttctagatacttattggcgaactgcccaccctttgcccccgtttttttattaatcaagcagcgctgcattttccaccattctaaatcttcaggagaaagctcaatgccatatatcaactttaacgttattgcatctttttcaatatccttatcaatttggctgagcttttgagctttaagcgggtctagtgtgtacttccatttaaacttagtgtcctgtagtttggctacatgaaatacggaacatttcggcggggcctttgtgacgcccttacactgcggaagtttatcattaggacaggcgcatagatgagactgcgccacagcatcgcgaactacatcgcagacggagtacattttcctcctatgttaaacaataaatttttttcatagctgaaatttgtgggcctatcttttcccttgcccggataataattataagggagtgttgaaacatctgggagagaattgcttaaaaaatgggtttttgggaggggtaactgcgactgttgtacgtcgttggccagggagattctatatgccgggctaaaggtgcaacgttcctgtgaacaacttagtacgcgcgttgttaatacaaatggactggtattagcaaacctcgtaaactcttccggacttgtttgtttttgtatgatgtttagcagggagtctgccttttcgagaatccaaagcgtcgcattgtagtaaaataaaaatagcgacttatcggcaggcgttgcaaaagcgccgtatagaaaataaagcagtaagtactggggagacaccacaataaggttatcttgaatgatagatatcgctagctctttaaacatagtgctaaaaaaatgtatgtcgttcgtcttgaatatagggggactatagtccatgtagggctcacatatctcagtcaggtgaaggcccatttcttttatgacttcttccgggttgtacgtcgctaacaccagcgcgggataggctttgggcatatccacggtaagtgttatgtttttatcattcttatggtaggagtaagatggttgtggaaattctgttttccactccgggactttgcaggtaattctcagctcatttagagtctggtacaggagggcgtatgccgcaaagccgtgtatggccacttgtttaaagggaattgaaaacgttttactttcgtatgtcgacttcacaggaacaacgggaatggggtaatatttttctatgaggttataccgctgcaaatcctttttaaacctgctaaaaacatcttcccttggtgggttatcaaaaggaaagcaaaatgctaggtgtagcccggcccgctggtaatcggggtgaatgattttaaggtttttatacgttaatgtgggtatggtgttaaagatattggggggcatatatgaaagatcagcaacccacacaaagtccgtgcgcacccgcatggtctgcacatggatggcgcgcaccgtgcccacctgcttgaagcccttttcatacaaaatgtcagcaagttcgtaggcgtcctcaacgtggttgggggaaaacatatcaaagtcgggtctttctccctcgggataaattgagctgcctttaagatgcagggcataatcaatggcaatccccccgtacaaaataagctttttctttatgataaattcgcggaccacctccaaagccgcctcaatctccacggcatttgcctcacgtttttgagcaatgagccggtacttagaaacattaaaatcagtctttagtaaagacgtcataaatagtgtttaatatatattaaaggtttgaataaaatactaaatagtaaaaatggatgccctattaaaggaaatagaaaagttatcgcagccatccttgcagaaagaaaacaatgatgtatgcgatctctgttttatgcaaatgaaaaaaatttctaactatcagcttttatgcgaagagtgcggtcagctgaaggactggtttgaacctgaatataatgaaaaattcacggtatattctcgtctaaagatcgtgggtgccaatagttcctatcaccagcgcgatttggacaaggccaactcaagtgactatagctccttgcaatttcatcacattttagaggagctcaaatccctaaatgttaagtatatggatgcggggcaaaagccctttcctattcaggtgttaaaagaaactgctcacagttataaccaagtacaacaacatcgggtcatacgcagcattacaaagcttcagatcttagccagtattctacgtagcatttgtttaaaattaaacattgcttgtacggtggcagacgccgcgaggtttactcaacttaataccaaagggatctcaaggggcatggatcttctgcgctccctatttgtagacaataaaattactttaaacgttgatttaaaccctatagacagctttattaatagtacctacagtgccttacaaattaaacaaatccaccaagaactgcaggaggaaaatgtttataatttaaaagaaattgttaagagctttatattatacgcggatgagaagaacatcggcgtcgatcttaacaggagaaccgttgtgattgctacgatgtataatgttttacgccgtgcctactaccccatagaaattgatacggtggtgtatcaatgtaaaatacgaaaaaatacaattacacgtgctcttaaaatgtatgaggattactactcccactttaagtctctttatgagcagtatcatttaaacgcggcaaaaaaattaatttaaactaaacgtttaaactaaatgtttaaactaaacgttaaaactaaacatttcgactaaagtttaaaacctagtctaacagcgggatgcccatttccctggggttccatatttcaacaattttttgaccttcgggtgttaccttgatgcagcgcatgacgagcagtggaattttcctattaaagagttcttgcttagctatatcaataggactgctatatttttttttaagcattgtagatccattaattgccaattgttgcgctctaacggcgaccaaccttgtggcctcaaaggtggttaaaacgttggaggtaatgcgctcgttatcgggtataatgaccaatgtttgcgacgaggcctgcacaaagccctcgcagatggacggagactccacgatctcgtccttgtcctcggactcctcctcactgtcgacgaggttctcctcttccgtttccacatattcctccacgaggtcatccatgataagatcctcgttgtcattatcagccatattacactgttatcaaatgtactgtttaatacgcaaatggatttactacgttttaattgtatgtcttcatgtgcaggctctagtggaaagtaattttctcacaatttttggcaccgttacacttgtgcccacaaaaacccgcgatttttttattttatattacttttggaagtacgagtttaaccagtcgctttcaaaccttatgcgtctatctcgccaaaaaacgctcacagcggtgttggatattacctttaaaaaaataacattaatttttaccacagagggcgtattgcgtatggattctacgaataagccaggcgtgccactcgatatagacccccagttcattgaccttgatagtattttaatggaactggatcattaggacctctcccgcccatttaaatttttagtttctacaataataaaatgcgcgaggaatcatgggaagaccacgataccattcagctcaccgctcagcgcaaatacctcgccgaggtgcaagctctagagacccttttgactcgagagctttcagtctttctcacagagccaggcagcaaaaaaacaaatattattaatagaatcacaggaaaaacctacgcacttcccagcacagagctactaagactctacgagcatctcgagcaatgtcgcaagcaaggcgccctcatgtattttttggaaagacaggggacctactcgggtctcatgttggactatgaccttaaactcaatacaaatgctgttcccccgctggaaccccccgcgctatcacggctttgccatcgaatatttgtgcatataaaaaacagcagtgtgctgcctgagggcagccataaaatccacttcttttttacattaaaacctgaagtggttcagggcaaatatgggttccatgtgctcattcctggtctcaagctggcggcttctaccaaaaaaagcattataggatccctacagcacgatgccaccgtacaaaaaattctacacgagcagggcgttacaaatcctgagtcctgtctggacccccactccgcctccgttccctcgctcctctacggctcctccaaactaaaccacaagccctaccaactgaaaaccggctttgagttagtctttgatagctctgatcccgactacattcccattcatcaaataaaaaatttagaatcttataatttagtttctgagttgagccttacgaatgaacagggaagccttgtaagacctgtctattgcgcggcagacattgccgctgagaaggaggaagagatcccgaccgaggatcactcgctctccatattaatgctacatgatcccgaagcccggtatttacataaaattttaaatctgcttcctccggagtattatgtagagtaccccctatggagcaacgtcgtattcgctttggccaatacatccgctaactatcggcccctcgccgaatggttttcgcaaaaatgccctgaaaaatggaatacgggaggaaaagagaaactagaaaaactttggaatgatgcctcgcaccacactgaaaagaaaatcaccaagcggtccattatgtactgggcccacaaacatgccccccagcaatacaaagaaattgtagaacaaggctacttttccattctcgctgaatatgtgtatagctataacggcatgcttgagcactacatgatcgccaaagtcatctatgctatgatgggcaacaagtttgtagtggacgtggattcaaacgggaagtacgtttggttcgaatttgtgctaccgggccagccaatgaatcagggagaaatatggaagtggcgcaaggaggtaaacccggatgagctgcacatctatatttccgaaaacttttcaagggtgatggaccgaatcacggagcacatcaaataccacctcagtcaaccccatgaaagcaatattttaaattattataaaaaactattaaaagcctttgaacgctctaaaagtaaaatctttaatgacagctttaaaaagggagttatcaggcaagctgagtttttatttcgccaaagaagctttattcaaactctggataccaatccccacctactgggggttggcaacggggttctctccattgagaccatcccggctaagctcattaatcattttcacgagcatcccattcatcagtacacacacatatgttatgtgccctttaatcccgaaaacccctggacaaaactattattgaatgcactccaagacatcatcccagaacttgatgctaggctgtggatcatgttctacctaagcacggccatatttcgcggcctgaaggaggctctgatgcttttgtggcttggaggcggctgcaatggaaaaacttttctaatgcgacttgtggccatggtattgggcgatcactatgcctccaagctcaacatcagccttcttacaagctgcagagaaaccgcggaaaaacccaacagtgcctttatgcggcttaaggggcggggatatgggtactttgaggaaaccaacaaaagcgaggttctaaatacgtcgcggctgaaggaaatggtaaatccgggcgatgtcaccgctcgagagcttaatcaaaaacaggaaagctttcagatgacggccaccatggtcgccgcgtccaactataacttcatcattgacacgacggaccacggcacatggagaagactgcggcattatcggtcaaaggtgaaattctgccataaccccgaccccagtaacccctacgagaaaaaggaagatcctcgctttattcacgagtacatcatggatccagactgccaaaacgcattcttcagcatactcgtctatttttgggagaagctacagaaggaatacaacgggcagattaaaaaagtgttttgtcccaccattgagagcgaaacggaggcgtacagaaagtcacaagatacgctacataggtttatcacagaaagagtcgtggagtcgccctccgcagaaactgtgtacaacctatccgaggtcgtgacggcctacgcggaatggtacaacaccaacattaacgtaaagcgccatattgccctcgagctatcccaggagttagaaaactctgtgctagaaaaataccttcagtggtctcccaacaaaacgcgaattctaaagggttgccgtattttgcataaatttgaaacgctgcagcccggcgaatcctacattggggtgtccacggccggcacactcctaaacacacccatatgcgagccaaaaaataaatggtgggaatggtcccctaatccctctgcccctcctgagaaagaagcgtctgcaccaactccttagggaatatccttagaagcatgtctttcggcagagccattaccggtagcaaaaaagcaacattgagtatattatatgccttagcctgctcataagcgtccttttttttcatggtattttatgtttttaaatatttttaattattttttaaatacgatgaacagttcgtgctccgaaggctgtttactaaaaatcggtgtgaatccgcattctttaaatatggtttcccattcggggatggtatggaaatccatgtctctacgaatagtatggtgcccaagtgcgtcctgcaggctgtgaagccagaaggcctcctgaccttgatgaaggtcgtacatgataagaaaaccatcaggtttcaacagatggtaaagcttgttaaaatcgtttatcgtaagatgatgcgccgccataggtaaccctatgagctccacagagttttcatgctggacatcgtccatatcggtataaaacgtttcacagtaaatgagacgcttaaacgagtatcgatgacaaacatttatttccaagtaggtttgcactacgtttttaggtatatcgggaatcatgttgattaaggttgtttcgggaaacttaatcatctgactaggcttcattttcaactctttaaaggatttcccggagaagtgaaaatgggtctttacgtatttatgtaaaaatacctgaatgggcagagggggctcctcctcttcgttctcgacgcctcccaaaatatttggaatttcctgacgtggcaaaagaaagtttatgtccacgtttacgaatccatcgaggacggacacaaagcttggctctaatctccattccatatactgtttagaaacgggagatagcataatcctaggcgtcacaatgcacgaagggtttttaatcaccgcatcgtggtaagaaaagtgtattccatttcttccagtataaagaagcctatgttcgtcgtagcagaaacaattaaggcggtatgcctcatacatacactgtttcaaagtacaaacacgttttaaaaaggtttctgcattggcggaggccaagcggttttgccattggtggaaggggttcaatcctacaatggccagctcgtttaaaatatcttcgcggcgcgctaaaatctgcaccatagaagaatactttagcatttttttttcgcaccattcgcgaagatgtttagctacattattaaccttattattgataaagtatacgatggcatgttggaagccttcaaaaataaagagcccctccaaaagatcatctgccaatagaagatggatgttggtgtaagcattgtcaatattttgtagaaacggcggaatgcctgccaaaaccgcttcagcaagcatagctccgttccgttgtttactgtccaatagattcgtaagttttttgtccgcaacagacacgacggctaggatggttgcaatgtcagaaatggcggcttgccagaaataacccgaaaagcacatgcgcgcttcttctatagataaaaacgaaaagcgagaggcaatgtctccgagctgcgtgagttgaagacctttttctcctctggttaaaaggcctgccacaatggcccgctcaatggctgatgccagcgcatccgtggggggaggatccagcatatcaatctcctctgccttaaacacgccttccttattttttttaatcgtttctacgacaatgctaagaaaaatggccccagggccttccgtaatgatttcaggatactgctgcactggtatttgctcaaagacgtgttttgtgtaaagcgggtaaaagtgcccaggaaatactctccctacacgcccctttctttgctcgatacggctttgagccgcggggcgcgtaataagccctcccgcccattcgggatagtaggtttcaatgcttctgttccacccgggatctatgacgtacttcagcgtttcaatggtaaggcccgtttccgcaacaaccgtggaaacaatgacccttcttaaaggtttttccactttagcggttaagggatttttcacccacagattcttaatttccgctttcaggccaaggtaggcctcattttcctgcgcaatcgcctcactatcgatcggcaaaatcaacattaacggcagcttttctttggcaaggtccatatttgcattattcagcaacatcgaaaggaagcgtatttcagccataccgggcatgaaaattaaaatatctgcttccgtgggacgatcatgaatgttttctttatgaatagtgagagccgtttcgcaggcggtcttaatgtagttgttggtgttatacagcggccagtgggtttccacaccgtactgtcgtccttccaccaaaataatgttttcttttccgataccaaaataggttgagtatttatgggtatcaatggtggcggaggttaaaattacaaagggaatacgcagcgcccctatgcttcctctttgcaacatgcgctgaagcatacttttaatatacatgagcataaggtcgatgcctagggctcgctcatgggcctcatctataatcataaaggcatagcgggaagctatctcatcatccgtcattgtatgtagctgcgccaacagaacccccgcggttgcataaataaggccccgattgggtttttccgtcagaggcttcgtttggtagcccactgtttggcctaatatcatgtcggggtagtgggttgaggcgccgatgtctttggcgagggtcaccgcggttaggactcttggctgggtacaaataaccgagcgtcccaagtatttttggaaagaatgcgtgttttcatttctcagaattctgaacacgtgtacgggtaaggccgtggattttccggaaccagtgcgtgactttataatgagcacccggtctgcgagggaggttggaatggcccctccaaactccgggagacgttgttttatccaagtgatgatgtaatgaataggaacatcattcttgtgctcagcgggcacgttatagagatgaccaggctccaataaagtcggttttcccatattctattgttttaaggattgattgttcataaatatttttatactctgaccaagaaattatttttttattaagccggttatttacgttgttatggaacgcgaaggtccagtactgaaagtcctccgagttgtttaatgtcaagggattttttgtaagatacgaaaaggcgtggtgctggcacctggtgcatggcagagactcgataaagttcagtatccattggatggcttcatatttttctttccagctaggagcgtctgaaaaaaagatagcatatagatgcaaggatcgccagtatttaggtccccaatgcaacatttataaccttttgaaaaatctcattccatatagaggtaaatattttttttccatggagaatttttttgcactcttgaagggattgcgccacatcgtcaaatgttttttgttttccatgtattttggcgtaattccagccagtatctgtgtcatggtccttaatgtcatccgctaactgaaaggcatgtccaaaacaatgggcagccctttcaatcatcccaatgtcttcaacggatccagttcctaaaacccagcccataataaacgcgatcttaaaaaagggaatggttttttctggagtgtctactaactgaccggaacccgcgctgtttagagagtggcttacaaaggtacacagcagcgctcccagttggttgggatccggaaaccttggacagtgttccttaatccagtcgatttgccggcaaatattttgaaatccttgcatggttagcgccagagcgctcatctgcgccttggctacgccaaagcgggcccacactgtatctttatttcgccgcttcacatcgttgtcaaaggagggcatatcatcgataatcaaagaagctacgtgaaagtactccgctgctagggcggcctctgccggataaataggcgccccaaaggaatgttgcaactgacaggcccgaacaatttccatcaggataatgggacggatatacttcccacctcttagagcgtaagagcaaggctctgttagttgtcccttaaagtccccatcttcaatagcattatttaagatggtctcaaactcttcactaaaggttttataatttttaggattcagtggatgtattccatgaaaaagcgcgacactacgcggtgctgtgattctaaaatacttaggtttgcgcgtataggatattaaaataataataagaactacaatgatggagatatagatgagatgcaacatgctgagttgtctccccgcagggaatggtccttttccgcgcttgttaacggtaccgaggaggcgttgaaatctttaggaaaggtgctgtctagtttggaatctccaattcctcccgtatatttaggtatataattattgtgtctagaaattgtttgctttgaggtatcaaaatattcagcctgaccgctatttcttttagaataattcggtatagggcttgagtagttggcaatactcttaaaccggggcaccaaggtaacaatattttccatataatgggtttgatacgctttgtttaaaaatgggcttaccggctttatgcttgttagttgtgcattgagtaccggtatgtcttctaggatttgtggctttatagaatgattagcaaacacagaatgtagtatattagatacttgtagcatatgtctatttgcggaaaattcctggtattctctgccgtgttgcgaatctttgggcggaaggggaccaagcatcggcacgtccgtgtaggtactggtggattttatgagttcctgctctatgttcggtttgacatgtggatttcctaaaggaatacctctacctgcaatccctttttctaccgacgcaggtagattgtgcgctaaacacaaaatattgtacacgtctttgtgcggaatatatccgttatagtgctggcccggcatctgatcgccaaggtgctgctcatgcttaatggtaccctttgttctgagtttaggaagatcctcgtacgaaaaaaattttgtgtgctcgctgaacctcgtagaaggaaccgaactattttttgggttttttaaggaaggcaatgaggaaggctgggtcagacaatttttctgtgtgccctttaagctagccacctgcggaaatgtttttttttccgtacgaacaacattgcgcctaattaggttttccgtatgggttgaaaaagcaggacgatgatttttaaaatgattaaaaagtttattttttggaatggagctgtacggctccagatcttgcgcatcgccgtaaccaatgtttttgtgctgagggttcagcataaaagaaaagttacgtagatcactgagttgcaatcccttttcagccttttcaggactattagtgtattcattgtatacaggcgcagctccatttttgttgccgcagtaccgggaatttagtatattatcagaataccggttatgacgcggcaaatcgctttcccaaagaggtggatctgacctataatcggctaacagctttgaagcataatcatgatacattgtatataaaagttaattattatattgagaaggcataattacttcttgtaggggtacaagaggctttgaatcaggcaaactgacgggttttgaatcggccggctttggaccggcaggtatctttttaggttgatcttcttctagctcattagacacggatgggggagaaataggaggaataatttcatctccgcccttatatttgtcatggatagaagaaacaattacatccatgtttgatttattataaatgtcgtttaactggtgatttaaaacataataatgcaaaaataatagggctacaatgcatatatatacgtaaatagccgtcttcgtttttcgttttttatccaccggcggattacaaattgcaaaaaatacaactaataccaccgctgtaatgattaaggccacaatgaaaggattttgaaaggatgttttgaacggttcgcacgtataaattttttctcctaaattattgatacccgcaataaaatctacattcattttatatatttataaattatgaaaaatttagagttacatctccgccggaccaatcattgctaaaatttgaagattcttcaaaaaggcccgactggttgaatgtcttctgctcaggtttccaaaaattttccaagaatggattttgaacaataggctcatcttgattttcttcttcaaggatattttctttgatatcaagaacagcttctttaaactcaggtgtatcttgattaaactcaggtttatcctgatcaatcgcaaaaatattatcttcttcagatatatcctgtttaatcgcaagaatagtttcttcctcaggtttatcctgatcaatcgcaagaatattttcttcttcaggtttatcctgaccaaactcaacaatatctttctcgctaaatccgtttttagtgtgaagctcttggttttgaagagaattatcaaaatctattttagttgttgtcctagaccgtggcacgggatagttatctaatggtttacttactatagtcctcgaatgtggcacgggataattgtttggtgacttgctggttagctcttggcttgttaatagttcttgttttctcaataattccatctctactacttctttttgatccgctggtgtctctttttggtattcttcattagaaaaatgttcagagggtaatgtttcaataaactttgtgagtggatagctgctctttgatgtagaagagcgttgaatttgctgataaaggagttgaacaagtcgccggtattcactctgtcttttttcatattttttacgtagcgtggagagatctgctaagagcgacttgttttcagatgttaattcttcaatttgatgaagaaggctgcgattgtatgaactaagtcttgcatacgtttcttctaattctgtctccggctccacataggcctgttttcgcagaaatttattgtatagttccattctttttttgagcagaaaggtaagactataatcttgcatttctttcgtaactttatggtagttttctttccggtttttgataataaagggcagcattttttctgttgtgataaaggtgcccagattgctaatgtagtcgcacagtagcaattccaagatagattctttcttttcaaggcttatagattggctgtattctttaggtatgaaagaatcaacaatcgttgttacgaagtttgaaaagtttaatgttttgctgttaatttgggtaatgttacaaaaatatttgtaaaaactatctagcattttttcataaagttttttattttgtttaacccctaaaatatagccctttacttgatactgatattccgtaacaatggaatgttttttgtatagtgcatttttgtataaaaagttataaaaaatgttgataaaatacgcaccaagggtttcaaaaatacttataacgtgggattcttcctgatccattatatcatatgtaatattattttaataaaaaattactgacgaataacatgcaaaaaaaatatgtttaaacttattttaagctagcacttatttaaaagtgttttaaacacgttttaaattgtatgttaatacacttaaaaattaagccgaaatttgctccaataaggattacttttatcaatgaccacctctttactataaacggctttacataattttaataatgctttagagccaaagctgaaggcagtgggaagcggcactgtactatggtaaaaatgttgccgatgttcatcctcgcggatgtacacaagtttcctatatcctttaaacacaatatggctaatttcttccacatactccttatcctgtttggaatagcggttgctttgacgggaaaaattcgacatacaaatagaggcatttgtaaaaatggaaacaaatgcgtttttacgaagattggcgggtaaatcggtatcatcttggcagcaaataatcatcgaaataaaacagtgacgattttggtaaaaaaactttttaaaaatttcttttgtaaataatgggtgcagttcggccgcgcagtcgtctaatattaaaagtaaacgaggattaagattgatatagtttaacgtaaacttttcatcctctgtaaggcataagtttttatacatatgaatgttctgtataataattttttttaaaagttgctgataaagcgatgtaatcttttcttcttttttttggtccgtttgttcagcctttaagcactccacttttgcaatatttttgttttccttttgctgtatatcgatcggaagtttatgatacaatgtttttagcatatcgatgttgtttactcgactgtagatggaggacatcatagtttgccgctgccagatggcctccaaaaagcgttcagcgcccttgttgtcattttttttttgcttatcggcgagccacaagcggtagtgtattagagttggatgtacaaaaccctcatatgaacgatttgagggttccgagggggcaaccactaaaatttgttcaatatggggttgcaggattttcataatatgtttaacgtacacggttttgcctgtttttgaggggccatatagcacagttgttttatctataaaatgatgtgctttgaactgtagttcaggaattagcttccctgaatgggtcgttagggccatctctatattattacaattctgcttttgtatataaaatttctttttcgagtttattattattgttgacccacatatctacccgtatcgtatcatcaggcacattgagcatttcaagcgcattatctaactgtttttttgtttttatcagctcgctttcttcatcgggggttaaattttctttactaagcagttgcttaattttttcttcgcagtcgtctataaaatcatactctcgagcttttttgatatttccagatgctttttctaggttttttagctccttaaaggaaagcagtcccttaatcccgctatccgtgtgaaaggttgaattatagatggagagccccggagcatccgggccagtttcttgtatattttttgcttttttgtggtaaatagtatttcgtaaaatctcttttcctatctttaggtcttcctcatgacggtccaaaatccgttttattatttcattattttgattaaaataattgtagcgctctctgttggccttaaagcttcccaggagtgtccagttgcctaattgaatggatgaaacctctgagaaaatctggtctttatatttataataaaattcatcaaccttttgttggttgctgctatccaccacatcataaataatgaaggcaaactctaggtcgggtttttctgggtagatgctttccgtagcggcccgcaactcttcgtaattatcctcaatgtaataattccacttataaaaagtatcctgaggtggaatatgctgcgaaagatatctagtaatttttgtgttaaagagaatgggtttaaacgccctcggattttcaagcatatgtttaatgctttggtgaagttctatattttgtaatatgtgggctgctgccctatagccctgtggggtttgggtgattgcatcaatatcggcctgaagctcattaggcacatttaatgttttttgcatgatgtgtaaagggatgcgctcaggatctgctaaatcggtgtattctgtgcttgtacaagtgcttgcacaggtatctacattggtatctgcacacatgcttgcacaggtgtctacattggtatctgcacacatgcttgcacaagtgtctacattggtatctgcacaagtatacgcactttgagcatgaagattaggatcaaacacaaaatgttctcgtaaaaagctatcgatcgttgttttagcttccttgcttttctgcgtctgggttttgcagctatctgctatagataaaattgtatttactaccgattcagagggaacatcattagtttcctgtttcaaagtatcaactaacgttattagctcactgagaagagttttggtcgtgtgggtaggttttgaataggaaggcatccattcctgcagagctttgaagacatatccaataaagctagtcattataagacgtcgaatatactgctcccgcaaatttgtaaaagagcaaaaggccaccctgctatcatttttgaactgtttgtaagggttcgtcctttggtaaagctgtttaagcgtttcttcggatatttcagtagagggatcctccaatacgtttttgagaagctcatcaatattaaattctgccatatcttagagtttattatatacatattaaagctttaatataaggggggtataacaatggacgaaatcatcaataaataccaagctgttgaaaaactttttaaggaaattcagcaaggattggccgcgtatgatcaatacaagaccttaattagtgaaatgatgcactataataatcatatcaagcaggagtattttaactttttaatgattatttcaccttatcttattagggcgcatagcggagaaacgctgcgaaacaaagtaaataatgaaattaaacgtcttattttggttgaaaatatcaataccaaaatatctaaaacgctggtaagtgttaattttttactacagaaaaaactttcaacggacggggtgaaaacgaaaaacatgtggtgcaccaataatcccatgctgcaggtaaaaacagcccacaacctttttaagcaactatgcgacacacagtccaaaactcaatgggtacaaactttaaaatataaggaatgcaagtattgtcataccgacatggtgtttaacaccacgcagtttgggctgcaatgtcctaactgcggttgtattcaagaattgatgggaaccatttttgatgaaacacatttttacaaccatgatgggcagaaagcaaagtcaggtatctttaaccctaaccgtcactatcggttttggatagaacatattcttggtagaaatccagaacaagagttggggaccaaacaagatccctgcggaaccaaggtgttgcaacaactaaaaaaaattattaagcgcgataataaatgcatcgcgcttttgacggtcgaaaatattcgaaaaatgttaaaagagataaaccgcacagacttaaataattgtgtttctcttatattgcgtaaacttaccggagtagggccgcctcaaatatcagagtcgattttactacgaggcgaatacatatttacagaggcaattaagatacgggaaaaagtgtgtaaaaaagggcgtattaataggaattattatccgtattatatatataaaatttttgacgccattttgcctccaaatgataccacgaatcgacgcattttacaatatattcatttgcaaggaaatgatacgctagctaataatgatagtgagtgggaatctatctgtatggagctccctgaaataaaatggaagcccacagatcgaacccattgtgttcattttttttaaagatgaagattttttagatgattttttttagttttttaaaagacgaaaaaattttttaaaagatgaatattcttaaaccccgcaaattacttttttttaggtactgtaacgcagcacagctgaaccgttctgaagaagaagaaagttaatagcagatgccgataccacaagatcagccgtagtgatagaccccacgtaatccgtgtcccaactaatataaaattctcttgctctggatacgttaatatgaccactgggttggtattcctcccgtggcttcaaagcaaaggtaatcatcatcgcacccggatcatcgggggttttaatcgcattgcctccgtagtggaagggtatgtaagagctgcagaactttgatggaaatttatcgataagattgataccatgagcagttacggaaatgtttttaataataggtaatgtgatcggatacgtaacggggctaatatcagatatagatgaacatgcgtctggaagagctgtatctctatcctgaaagcttatctctgcgtggtgagtgggctgcataatggcgttaacaacatgtccgaacttgtgccaatctcggtgttgatgaggattttgatcggagatgttccaggtaggttttaatcctataaacatatattcaatgggccatttaagagcagacattagtttttcatcgtggtggttattgttggtgtgggtcacctgcgttttatggacacgtatcagcgaaaagcgaacgcgttttacaaaaaggttgtgtatttcaggggttacaaacaggttattgatgtaaagttcattattcgtgagcgagatttcattaatgactcctgggataaaccatggtttaaagcgtatattgcgtctactggggcgtccagctataaaacgtgactggcgtacaaaaagtccaggaaattcattcaccaaatccttttgcgatgcaagctttatggtgataaagcgctcgccgaagggaatggatactgagggaatagcaaggttcacgttctcattaaaccaaaagcgcaacttaatccagagcgcaagagggggctgatagtatttaggggtttgaggtccattacagctgtaatgaacattacgtcttatgtccagatacgttgcgtccgtgataggagtaatatcttgtttacctgctgtttggatattgtgagagttctcgggaaaatgctgtgaaagaaatttcgggttggtatggctacacgttcgctgcgtatcattttcatcggtaagaataggtttgctttggtgcggcttgtgcaaatcatgaatgttgcataggagagggccactggttccctccaccgatacctcctggccaaccaagtgcttatatccagtcattttatcccctgggatgcaaaatttgcgcacaagcgttgtgacatccgaactatattcgtctagggaatttccatttacatcgaatcttacgttttcataaagtcgttctccggggtattcgcagtagtaaaccaagtttcggtacgcattctttgtgccgggtacaatgggtcttccaaaaggatctacaagcgtgtaaacggcgccctctaagggtgtttggttgtcccagtcatatccgttgcgaggaaacgtttgaagctgcccatgggcccccatctgggacgtgccctgaatcggagcatcctgccaggatgaatgacatgcacccaatatatgatggcccaccatatcatggaaaaagtctccgtactggggaataccaaaggtaagcttgtttcccaaggtgggggtacccgtatgcgggcgtactttattgtattcaaaccctactggaacataaggcttaaaatgcgcattaaaatgcaccaaatgtgtttcttcgatttgactcaaagtgggttcgggatcgggtttcccataacttttgttcacatttttaatgttagagatcctgctattcagcaagtcttgggccaatataatcttgtcggccttcccatcgttagcaataagacaaaaagctcctcctgatgccatatataatgttataaaaataatttattgtttttattaaatatggcggtttatgcgaaggatcttgataataacaaagagttaaaccaaaaattaattaacgatcagcttaaaattattgacacgctcttgctggcagaaaaaaaaaactttttggtgtatgaactacctgccccttttgacttttcctccggcgaccctttggccagtcagcgcgacatatactatgccatcataaaaagcctcgaggagcgcgggtttactgtcaaaatatgtatgaaaggggatcgtgccctccttttcatcacctggaaaaaaatacaatccattgagataaacaaaaaagaagaatatctgcgcatgcacttcatacaagacgaagagaaagcattttattgtaaatttttagagtctagatgagcttttacgcaatgttgtacagtgttgtatatatgtcttgtaagcatttgttgtagagtaataagtaaaagataaataaaaatgactattaaaataaagcccaaaccattaaaaatatttttatctgttagatttaatttaataaatggctcatggaatgtgtggtgcgccgctgcatgaggtgtggccgcatgggatgtggtcgcataagatgtagctacatgggatgtggcatttgcttgcatgtaaggatcatgatgtgttgggtcttcatcccagcaataatcgccatctttatctagctgaattgtataccccattatatatcacttattatttttttttaatgtttcatgaatttcattataggcggtgaaagggtcctcaggccccttctgtaaaagattatagagatcttcggacgctttatgtttcgtgcgaattaaggcgggatataacaaaagagagggccccagttccaaacaaattttacttagcgggctcatattttgcaccaagtttcccactacttgcgatgtttcataacgcattttaaagagctttatcataaaagtgttatgcaggccggtgtagtctggcctatagttaaggaaggggatttctctggtaccgtcaaacacgatctcaagtcctctagcaagcccgatcaaaatttcttcagcaatggatgagtatctaattcctacattacgaagcgtaagcatttctataacatcatctatttcctgcatagaggaatctattgtaggaattttaatatcatctgtgctgatttgttcattcccaagataggtaagcagcatattaattttttctagctttactagcttagtcttacgctcataatcatgatcttttttataaaaagagttgggatcaccgttggaccgtagatgattaataaggcggtctacttgctttgtactaggtttaatacttttttcactatactcgctttcagcatagtggtttttacgatctcttttagaaatagctgttttttgagatgcctcagactctgcatatttttttctatgcgtagaaagagaataaccgcggtcattacgtgaactactgttgcatgcaaggcctcggcgcgtcttaccgctgcgcacactgccattgcgtatactgccatcgcgcacactgccgctgcgtatactgccattgcgtatactgccgctgcgtatgctgccgctgcgtatgctgccgctacatacactatcactacatatgctgtcagtacatacgctatcgcggcgtatgccgccgtgtaccttatcgccgcccctacccgagggttttttagatataatactgtgtggggagtcaagcgaaaattcagggtcattaaagttaatgcccaatgactttgccaatccattaagctcttcatcaaaatgatcggtaggaaaactttgttgcttgcccatgacctgtttttcaagttcctccaaattggcttgctcatttatatggagattattcataagcgtcgtaattccagcaagatttgctccttctaaaaatgtggtgtcctccatcggatatactatactatttaaaagcttttaaataaaaatgtgtttggaagaaatgctctcttcaagcgtgtgtagctcagatataaatgcctcctcagaaagctttccaccatactcctttctcatcgtataggagggcgccggtttaatgtaggaaatccactgggaggtaaaaaaccggtacaacatatttagcagctcgcgggcctcccaccttttgggctccgtatagtgcacatcaacataagaggcggcgcatgaaaagctgcaaaagttgccgagaacgcccatctcaatctctcctcgctcattttcacgcatataggtgggcacgaattttgggacagtcttgaaatagagatgacatgtccagcatttaaagctagaatgggtaacccatttggaaacagtggtgaatacggagggtagctttttttcgacctcggcttcatcgtcattcgtatttaacgtatcggtggcagtttttttggattgcaagcattcttcaatggtaatcccggataagtataaaatattaggacaattagtttccataattttgatagttatttttatacaacatggatttaattaaagataaatggaggacgaaacggaactgtgttttcggtcaaacaaggtgacgaggcttgaaatgtttgtctgcacatacgggggaaaaattaccagccttgcatgttcgcatatggagttaattaaaatgttgcaaattgctgagccggtgaaggcattgaactgcaactttggccaccagtgcctaccgggctacgaatctttaataaagactccgaaaaaaactaaaaacatgttgcgccgtccgcgcaaaacagaaggcgatgggacttgcttcaatagtgccattgaagcctccattttgtttaaggacaagatgtataaattaaaatgttttcctagtaccggggaaattcaggtcccgggcgtcatttttccggattttgaagacggaaaaaacattatacagcagtgggtagacttcttgcaacatcaacccattgaaaaaaaaatccagattattgaatttaaaacgattatgattaattttaagtttcaaataaacccagtgtctccccgcgtcatcattcatttaaaaaaatttgcagctttgttggaacacatccctactccatatcccatacgtgaaataaagcctccattagaagactcaaaagtatccgcaaaatttatggtcagtccgggaaaaaaagtacgcattaatgtttttcttaaaggtaagataaatattttaggctgcaacacaaaggaatccgcggagaccatttatacgtttttgaaagatcttatcagcgtacattggcaagaaattttgtgcgtgttaccggtacccgattaaagaatgttttcattaataaggtaatcgactatgctaaaaagaataacaagaaaaataccttgaagaactataccaaagtaggtaggttttctgcatgtcacggcatggttaaaattgctaataatgtagtccacaaaagcattgctcaatacgactaaaaatagtaaaaaaaggataagtgctctttttatatccatatactttaaaacttattttttacactaataatttcctgcggccgcaatataaactgtaggtcatctataacgcccagacctgttaaaagtagagtactatgttttaagggatttaaaatatccgccgcaagaatgtgaatataattttcaaagtggtttacaggaatgcgtaagcgtttttttttgcactgcggttggtttagggtcgaatactggcaggaggtatatatattaataagaccgcggtcgatggtttcaatatcttcatagaattcaatgcgcggcgtcaaaagttttttaagatgttgacataactcatcatacgtgtaggactggaggggggaaagaagggtgtagtcaaagttaaaaatgtttttttgaagaacctttaaagcatgttccgcgtccgtggtttccaaaatatgttttatggtatgaatgtcatttaaatctacaaagtctgacagctttgtgtagaactcggtgacggaggttattttctggaaatcggttttttgaaaaagattttcaatgtgtttgcgggttgagttgctttgcagtccatacaagacatcaaaaaattcaatcagcaaaaacttatacaaatggttaatataaaaagctttgttggccttattctgctgaggatatggttcctctaggggatatagaatggcttggtctatatccctaggatcaatagtcaatgttgcgatgggaagcttttccagcgtagcgggaagagtttgggttggagcgtagtaaaagtatagcccggtttttccctctgaaagaaagcccacaaattctttttttatattttgcagcaccgctgagggtacgatttcgtactgtttatactgtttgttgaaaagggtaataaatttccaggtttcttcaaagcttgcaatctgggtgggccgcagatcaaagtcgatgggaatgtcgtcatgaatgtaggatgatagtcttataggaaaataaatagggcgatcggtgtctgaatcgataagtaaagcataacaaaagttatgcctgttgataagttttttaccaaccgtgtagccgggaatgtttttcacgtcatggatatcccaccagttatccttgcacataaactcgctcatagactggatgacctccatcacagggtcatcttcggtaaaaatatactgggcctcactgtttttcagaaatcttttttgctgggtgatggccattgggtagatcccttcgtccgtgtcaaagataatggctatcttcttcgatgggctaagaattttttgtattgtgctgggggacacctcaaacccgatgtcgccctgtttatctttaaaaaagacacagtgaaggtcgtagcatatggcaacaaggtccagaaagatgtcctgccatgtggtgtcccattgaagcagttggtttttttgttcaacaaaggtttgtaagataaggtttgccagctccgcgccgctggaaaacatgttgccggccccattccccaaaatatagtactgcggtgtgttggccgcctttgcaatttcaatggcaagggccttgggggcaagatccaaaattcgagcaagggaataaaaaagcccggcattgctaattccaagcatggtttgctccacccccacaatgcaaaaaatgtcgggctcttttatcgtatttaaaaacagttcatctgctatctggtggggtagaaaggcaatccggttcaccggtattttttttccataggacaaggtatgacgcgatgtttgtgtattaagatcctccaggtcttgttctacaaacgtgtgcttggtgaggcaggtattgttaatatagaaccgctttgtgcccagcagggccttcgtcttttggcagcacggcagacagtaatttagggggtggcggccttctagtaggcttagatgagggtagtcaggatgcgggcagctatagtaggcaggtaccccctccgtgaaattccaatactttactagctccttgcgcttggctggcggcatggacttcacctcggcctctgagtaaatgacgggtggccgtgggtgctggcataggacggagtaaaccgttgcctgcgtgtcgtacttgcgcaggtcatacaggtcggggtcctgttcttgaagcgcacgtagctgagaggctccctttccttgttgtttatcgtgcagttgagagagtttattaaccaaaattttgtcaggcccggtgatcaagttatctaaaaacacaaataggtaaacccaaagatagttaaactcttcctgggtaatgttaaacatttctattttgatatctgtaaccctatggtagatgcgaatgttgcggccgccgtagattgtttcccaccgggccgcaacatttgtgtcaaagaggtacgcatacgtgttttggagcaacgcaacattgatgtccattttgcgccccggaccggaggaaataatgatcatccgttcgatttcgtggggatcatacgaataaatcccctttttaaataaaaaattgtagaccccggtttgctggaggccccgcacggaaataatccctgcttgctcgtattcccgccaacgacttttgagctcggtaaatcccttgctagaaagcgtatagggccaaaaggtggacaccgacatggagctgatagaaatttggatgtcctcgttggagggaaggggcagactccctccacgaggaaacgcggcaggccccatatcattaattgtatgaataataggatttatgaaattatttagggtggacaccacggagttaaagtcgtggcgctcgttttctgaccaattgctttcgataaagtagtgcccattattttgtatggtaagaataaaggcctttttattgataaagcgtattaaaataatagtgggtacacggaatgttttattgctgaatttttcaggctccgtggaagttatgtggtgtttggaaaccacggtgggacctgttttactataaaagaacaccaccagctgaggaatatcgggagtagctggaaataggtcgaaaacattgcgcacattaatttgaatatttacgaggggtgaaattttaatcattgccgaggtgacggccaacgtgccgcgtgttagtctattcccctcgtacttggcaatgacttgttgtgctctggcatacgtaaagtttattagtttttgctctaggagaagcctctttttaagactggtcaaggatggagaaagagcaggatactgtttttccatttgtaagggagattgtaccaatagtttaaaggcatcgggggaaagaagaggccaatacttcataataaggccgtaatagagtaagtcaaattggtaattatcctctatggcaatggagatttggcgccgcatgggggccactagcgtgttgaggtctgctacaaagatgtgatgaatgttttttatgagctggaagctgtcgagcgcttccacatagagctcatctttttgactttccatagatgcgtcgatgttcaccccacccacctgttgaaactcctttttgtagtcgcgaatgtctaacgccaccccgctaccgcttaacaataggcgatacgttacctgaagcgcattgttttgaaaaaagaaaatgtgttgtctataaggggggatccctgtggcaacgtaaattttttctcgaatgtctttaaaagtgtcttcagggaaaatactatactcgctatacatcgtctcaatttctggcatcatcacgtttgtctcctcgccacgatcctccacaaaaagtttttcaaactcatctaaatcatcgctatctccacccaccacgtattgggaaagctttttctcccaatcctcgccgtaaaaattttgtaaaatttctttgtccttaggggttcgctgcaggtctttgcggcaggcctgtaacacgtttgcaggaacggatcccaaaaaaataaacgtcttcgtgtactcattttccacaggattataaagagtaactcgtagaggatttgttaaaaagtcattttggaaatccattatacccggtatagaaaataaaatttaaaataaaaaacggatgatatctatcatggaccgttctgagattgttgcacgggagaacccggtgattacccaacgagttacaaatctcctacaaaccaatgctcctctactattcatgcccattgatatccatgaagtacgatatggagcctacacacttttcatgtatggttccctcgaaaacggttacaaagcagaagtaaggattgaaaacatcccagttttctttgacgtacagattgagttcaatgatacaaaccagctttttttaaagtcgctactgacggctgaaaatattgtgtatgaacggctggagacgctcacccagcgtcctgtaatggggtaccgcgagaaggaaaaagagtttgcaccatacattcgaatattttttaaaagcctgtatgagcgacgaaaagccattacttacttaaataatatgggctacaacacggccgcggacgacacaacctgttattaccgaatggtttcccgagaattaaaactacctcttacaagttggatacagcttcagcactattcctacgagcctcgcggcttggtacacaggttttccgtaacccccgaggatcttgtttcctatcagaatgatggccccacagaccacagcatcgttatggcctacgatatagagacctatagccctgttaagggaaccgttccggacccaaatcaggcaaacgacgtggtgttcatgatatgcatgcgcattttttggattcactccacagagcctctagcgagcacgtgcatcaccatggcaccctgcaaaaagtcctcagagtggaccaccattctatgctcctctgaaaaaaatttgttgttaagctttgctgaacagtttagccgctgggctcctgatatatgcacagggttcaatgattctcggtacgactggccctttatcgttgaaaaatctatgcagcacggtattctagaagaaatctttaacaaaatgagccttttctggcaccaaaagctggataccattctaaaatgctattacgtaaaggaaaagagagtcaaaatctcggccgaaaaatcgatcatttcctcctttttgcatacccctggatgcctacccattgatgtccgcaacatgtgtatgcagctttaccctaaagccgaaaaaacaagcttgaaagcgtttttagaaaattgtgggttagattcgaaggtagacctgccgtaccatctcatgtggaagtattatgaaacacgagacagcgaaaaaatagccgacgtggcctattactgcattatagatgcccagcgctgtcaggaccttctggtgcgccacaatgttatccccgatcgcagagaggtaggaattctgtcatacacctcgctgtatgactgtatctactacgcgggaggacacaaggtatgcaatatgctcattgcctatgccatccatgatgaatacggccgtattgcttgcagtaccattgcccgaggtaagcgggaacacggaaaatatcccggcgcctttgtgatagaccccgttaaagggcttgaacaggataaacccaccacaggtctcgactttgcgtcgctgtacccctcactcatcatggcctacaacttttcgccagaaaaatttgtagcctctcgggatgaggcaaatagcctcatggccaagggtgaatctcttcactacgtctcctttcactttaacaatcgtctcgtggaaggatggtttgtgcggcataataacgttcctgataaaatgggattgtacccaaaagtactcatcgatctacttaacaaacggaccgcccttaaacaagagcttaaaaaactaggtgagaaaaaagaatgtatccatgaatcccatcctgggtttaaggaactacagtttcgccatgccatggtagacgcgaagcaaaaggcgttgaaaattttcatgaacacgttttacggcgaggcaggtaacaatttgtcgcccttctttctgcttcctctagccggaggagtcaccagttcgggtcaatataatcttaaacttgtctataactttgttatcaataaaggttacggcatcaagtacggtgacaccgactcattatacattacatgcccagatagtctttatacagaggtaacagacgcatatttaaacagccaaaaaacgataaaacattatgagcaactctgccacgaaaaagtgcttctgtctatgaaagccatgtctacactatgcgccgaggtgaatgaatacctgcgacaagataatggcaccagttacctacgtatggcctacgaggaagtactctttcctgtgtgctttacaggcaagaaaaagtattatggtattgctcatgtaaacacacccaattttaatacaaaagaattattcatccgcggaatagatatcattaagcagggtcaaacaaaactcaccaaaacgataggaacgcgaattatggaagaatccatgaaactacgccgccctgaggaccatcgcccccctcttattgaaatcgttaaaacggttttgaaggatgctgtggttaacatgaagcagtggaattttgaagacttcatccaaacagatgcgtggagaccggacaaagacaacaaagcagtccaaatctttatgtctcgcatgcacgctcggcgtgagcaactaaaaaaacacggcgctgcagcatcgcaatttgctgagcccgagccgggagaacgcttctcctacgttatcgtggaaaaacaggtacagtttgatatccagggccaccgcacagattcctccagaaagggggacaagatggaatacgtctctgaagcaaaggctaaaaatcttcctattgatatattgttttatatcaacaactatgttctaggcttgtgcgcgagattcattaatgaaaatgaagaatttcaaccccctgacaacgtcagcaataaggatgaatacgctcagcgccgagctaaatcctacctacaaaaattcgtgcaatccattcaccctaaagacaagtctgtcattaagcaaggcaatgttcatcgacagtgctacaaatacattcaccaagaaattaaaaaaaaaataggcatctttgccgacctttataaggaattttttaacaacaccacaaaccccatcgaaagctttattcaaagcactcagtttatgatacaatactttgatggagaacaaaaagtaaaccattctatgaaaaaaatggttgaacagcatgctacggctagtaatcgagctggtaagcccgctggtaatccagccggcaatgcgctgatgcgggctatatttacgcagctgattacggaagaaaaaaaaattgtacaagccttatacaataagggggatgcaatacacgatcttctcacctatatcattaacaatataaattacaaaattgccacgtttcagacgaaacagatgttgacgttcgagttttccagtactcatgtagaactgctattaaagctgaataaaacgtggcttattttggctggaattcatgtggcaaaaaaacatctgcaagcttttttggattcatataacaatgaatcgccgtctagaacattcattcagcaggctatagaggaagaatgtggcagtattaaaccatcttgctacgactttatttcctaatacttcttaagaaactctttaaacaaggacttcgcatggtcaaaggttctaaacccatggcccttatgattcgccaaaaaagcggtttcatcaagattttctaaccctttcacggatgaagaaataaggtgttcggcctcgtttgcccattttctatgattttttttcacctcgggttctagatctgttttctccatatactcattgtggtcatatttttttttgggaggaggcgtgggtggaggaatgggtggaggaagtacacccgactttcccgcttcaaccgttttataaaaaaatagaagcataatacaaagaataaggactatcgcaaatatgataaccagtgtcccagtcgagggcattttgttatataagtaacgtttttttttattttttataattcgaatgaagaaccatgttgaatagtcttctactcaaagacattttgttatacggtaaatgagaatttataaaatccgaatatcactatcatactgtttatctgagaaggtctcactgggtcctgtgatggagaacccatactctgtaatgctggggtttataatgtggtcaggactgacaagcacatttctgaactgcgagagttctaggtttagacgcagtcgtaatagtcgctgtatatttgtaataaatattagattgcgtatgaggcgagtgtcaaagcgatcctttccaatttgtactaaggtgggcttttgtattccaactcccacttgtttaacgatggaccagggtccttcttcccgattttgttccgtgatataggtcagcacactattttctgtatatgaggtatgatgtcgcatattaatacctggtgccattccaactggcggttgtgcaattcgggctgtaccgggacccaaccatcgtggagttttataaacatatcgttctagcgtatttaaaaattccttaaggttatttacgagtagcatgaagggtgctattaaaacaggtggatggtttataaccattgtcataaaccattgcattgcttcaatatcattttgtaatgcttgacggggaggcggggcaggtaatccacgtatgttgaataaagcggttaattgtgcaccggctgtttggggcgtaatattttgtattaaatttatcatcgaattggcttgcccggcatttcctataagatcgattaaattggttatttgacctcgatattgttgtacccagttttgaatggcagcgatgatctcaggggttggattgttttgaatttcaggtgtttgtattagattattcacttctcttcgtgtatcttcaagctgagtcctaaatgcatttaactcgcctataatttggtttctatcaataacatttcttaaacctcgaactgtttcagccaatcgtatagtacgcacaatttcatgtaaggcctggtttatgtatattgacatgggatggccccaccgctcacgtccacgttgaatacctgcggccaaactaggacctgcctcgtcataatcaaattgtgtaggataaaggcttccaaatagcactttattgaaaatttggtcagaaagaaatttagggcggcccatatttagcgcgttgtcccctctaaagatgcgtgacatgtatccggcgttgcctttggatagtaactcattcccatattgagtaatagagaccgagacataggggtttataagaagttttagcataaattctcgagtatttatggggggacgattcggaatgtttaatacctctgcaacatctggttgaggagccgtggtgtccagagatcgtactttttcagccgaaatgccgtacataagacaagcaatttcttcaaaactatagtcatagttgtaaatattggcaagtggtatagatcgcatcagcgcatttacattgataggtataatattcatatcaaacaagttaaatatgcgctcgcgctctctattagagccaagagtgcgtgtttgacctttcggcgacactattttgtgaatatgattgatttgctcctcttggtaagagctttccacgaaggaaattacgtcttgcaatgttttacgaagcgaatacactgcattcatccctattcccgctgttataatgggtttatcgtctctgttctcgctaataagattaactccaccaaaagtattttcattgtacatcatcactgttttaaaactacggatatttatgataaatcggagagcctgaatggcgtgggtataaaagtgttcaaatcgcgtgggagtaatttgttcgcgagcaactaccgtttcattatagtttttcatgataagctgtactccgggcatatctgagagctgtaccggatcatttcccagtaattttcttgtgccgtatagtagtttaaactcgggggagccgctttcaaggttcgggtaaagaagaggatcatatacctcattattttctattcttaggtcatgtaaataatagagcgaaagtgaaaatggcataagaggctccttattgtaccgggacatatagttttgaatgaagtgttcttctgtttcaagatagatgggatgatcggtaagctcgtgcaggacctccatggcagaatctgccagagtgtgagagcctctaatgatcccgtcgatcactgcgaccagtcgctttcgcacaacatcgctcgtattattttgtgcgtctcctaggggcataagcgtaacattgggacgaaatacgccgccaattccccgcagggccgcctgaccgacggatagtcctgtcgcaggaacattgttattattataataaataacggaatcattattggctcccaagagtgccgtcagattagggcgagctagttggacatttgtgtattgtataaattgttttagaagctctccctggctaataagaatattaaacattttgttaaatagtggaagattggctctataattttctttaaggtaaatgggaatttctgttaaagtagaaataagatgctgactcaggccctggcgattggtatccttaataagccgctgaagtataagtcccaaagacagaagaagcaccgactgctctgtggggtcgcctctatgaccaaagacgttgttattgcgtgctaagtcagggtgagcatatcccatctccatcactgcttggctaaagttcccattagcgaatgcattaataagatttagatatatttttccgctgggagcatcataaaatcgggtaatatatgaagctatgagctggttaaacaccatcatcatactacgattattttgaataccatagtctgatccgtataggcgataacgtcgaaggttgtttgcggcatcattgacattggcataggttctgagcgctatgttgtcccagtagctaagagtattttcctcctgggcgttgttggtacgaataagattggagagtctaaagtctcctagtgccacctgctctacacgaagtccagagttattctccaaagcatcgtaaaatacgagtctactgaatactcttccgtattgttcaaagcgttcagaggattggggattgttatttatttgaatattagccgcgtcccttctttgcgccccacctcgaagttgcagtacattataaggctttgtaagcaaggtgtaggttttattaatgatttggttaaccccctccaggcccaattcaccgccaggaagcggccttcctccggcatcggtaggtggtttaataagtttgtcaattaaatgttcttccaaccagtaaaatgagccaggattagatctattttcatagtattgaataatgtttttatcaatatgcgggcgtagaagatcaagaaaatacttcgtgtcggccatcaaagaatcaattaaggaaataagacctgtaaaatctaaatgcacttgagcggtgctggtttcagggaagcgaacttgaaccattttgttaaaactggaggtcatttcgaagatattggtcaacaggagctgcatgattcgctgattatctactaaataccttgcggccaactcttgctccggacgaactcctccaccagcaggaatacccacatatggtacaatccaagcaaaaagagtttctgtggttaaatttcggtcttgggctgctgcagccgcttcggtagtgggatcagggtacaccatagaaagccgcatattgatttctttaatgactaatcctggatttctaatctcagagatggccccgtgttttcttccgagccagtcaataagattggcgcggttcacgttggcagcttgtgtctctcgtaaccattcgataatgcttttttgaatcgtatctaggtctaaacctttaatgttattacgaaagttattaagaagtacgtaaatagcactcaataagttaagacctgtaataacggtttcatgaaacagaaatattttgttaacatctgtatctgccagtgactcagagccttgaataagttttgaaacgatttgaattttatcggtatgctcctttttgagttcattgatagcctggcgaatgagttcttggtaggaaattttgcccaattcttgttgcagactgggatcttcaaacatctcactaagctgtttcctaaatttttgtaccaaatcccactgggagttgggctgcagcattcctgtttggacatccacagagtctatattgtatagtgccgggcgccacttgggggtaggctgggttgaaggactaataaacctatcggagggaagtaattgtgaggattgtgtatagccatcctcatcaggaagaatggagtagttggtttgattcatcattccaaaatcattcatagttcgcgcttcctgaacaatgcgttgaaatttttcccattcggtgcgtgtaatgacaccgaatctgcggtttatttcatttacaaaatggataagcgcttttttggttgcttcttgttcaccatactctaagttaaagtgttggtaaatgacgtttatttctttgataagctgacgaatttcggtttctgagtagtcaccaatgttaataagctcaataggacgcataaagataatgcgaataagtcctgagaagattccttccagctcaggaagcatcgagatctgtacattttcatctctaaaggaaaacaacttttgataaaattcggcgaggcggggaaggcggaagtaaagctctgctgcctcgggaattacctcgggctctagctcatcggcaccccccaatatcatacgcgtgggtataagtttgtacacgggctcaggccgttcaaacatgtcgtaaatccctaatacaataaaaatcttggcggccatacttttcagcatgaaggtgaagaagacgtcctcggtttcccagcgggttgatagggcgtcgttaactctcacagtagagaggtagacccgctgagccgcttcctcggcagtctgtgcaagcgccatcctttgtcctccaatttctgattgatttagatttttaagtcccacggaaagcgcagaatgttgaagatattcaagcaaggttttatagatttgcaggggcgacatgggcaccatttgccgcagctcctctcccccaagcatgtccccaatccgggcaaaggcattgatgatatttttaagcgcctgaaagttagaaagagagcgcccgataaggtcgcgaatgtttttagcctggcttgctctgacgggacggagggtaccaacgcttcggccttgttggatttcagccgcaactttttcgtagtagtggcccgcaggagcattatccgtaaagacgttggagtcgttgcctgtggaggtgggaaaactttcaaagacttgtgcaagcgtgtcccctgttgtctcggtgaaccatcgtcctataatgcgcacgccatccagcatctgttggactgtttgaatagaatctatgttgtttacaaacgttttggtaatgtttttaagataaagatctagcccttccagagctcgatagaatcggcgttttacatcatactccagctcgatggcgcttacggttgccttccagtctacttcctgggcacctccaggatttgggcccacgtgtcctctggcaagatctacagccggagaattaatgcgcgcatttttttccgtatccaactgcatgaggcgtcccgcaatagcatctccgagaatagtggcatagttttcctcgtaggattgaaactcctgtttgttatgcgttaaattggagtaaatctgggccacataatagtaatacataaaggtgttaattgcctggttgaggtcaacctgcgatcgcgcggccttgctgagcccaagctcttcaactgttagggcagcaccgcctacccttgtacactcgcagtcctcctcgcctccatactttttttgcacaatatcggtataaaaatcaataatctgtagcaagcgagagcaggagtcataaagatttttaaaattagggtcggttttagatatctcctccaaaacatttttaacaagcgtaagctgtgttaagaaggtttcgcgttcttctcgtgcggccgcattggtgtaaaagccgataagacttagatcaagtgcgatggtgcccatatcattaatgcgcgaaagagcatctcgaagcctcgttatgttcggcgtcaaggcaatttctttaacaagtttgatgcctatttttttcacattttccaaaaagtcgttataggcttgtgtgcttttattcaaaaattccatgaggatgtgctttctatccagtctttgcgcttcaatcctcctatctagtggcgttttctcctcatcgcccccctttttggcacaactgttctcaaggattttgtggcgttcattaaaggtctgtcgcaacaggttcacggctttttcaaactcagcaatgttttctgcggagacaagaccactaaaccttttgaggtcaagctccttgtcaaactccgcccagtttttgctttgaaggtactgttcaaccttgagtcctactttctggagagccttattaattttattcgcaacagacgcagcaatacctagattacaaagtgtgtacgaaagtacttttccaaaatttttggttcccaagacactatttgtatcatttaaaagtttaataatatccacctcatccgtctgcagtttatcaagttccttttgggtgggagttaaaatattgtcaataaaattcgttaaaatgttgatttgcaggttttgttcatttaaaagtcgacgatatactgcttcaatcatggtgactgcattaatgacttcctcattgggggctgctttggttacctccgtcaccatgcgctcgtgaagttgcttaatggcgtcgtttaacagcttgatattttcaagtgtattttctatactgccgtgtacatcaagatactctgcgcgcagtccatgagttagggagttaatgtacagaactatttgtcgacatatactggcggccccttcggtggtatctataagcttatcctgacctaaatcaataaattcctggttaatggcgtctgcaatcattttacagacggtctcctgtttttccgcattttttacaaaggtggaaccggctcgaggatcgggcagttgttttttgatatctttaagaatatcttcgatgggctgctttgtgtctactttgaaccctattttggcaatcgccctgataattccttctataatccgcagctttgctttactcgatacggagtctatgtgataatctttaatgtgttgtacaggatttttgtcccccccgccattaaaatatcctccccctgaaaaaggacgagtttgtctttgtatatgatcctgtaacttcgcatatatatttgcttctgatgaaggcagtggtctactagaggttgaagatccacggttacccattataataaaaaaaaataaagatttaaaactacaaatattttgctgtttataaacccaatcatataagactaactaaaacattaaatgtaggtgagataaaagcttattttttttttaaaagtttaataaccatgagtcttaccacctctttttcttcttcctttagaggggttccataaatggtttgaataaaattatgtgctctaataaccttgttaaaatcaggtgcctttccatattgttcaatatgttgcacagtcttttgtgcaagcatatacagcttggagtctttaggtacctccgatgagggctcttgctcaaacaacgtttcaaaggaggatgtgcattcattggtttcattatcatttttttcatgaatgttctccgaagatgctgaggattccgtctcctcttcaaacagcacatgcagaatcatattccattcttcttgagcctgatgttcagtatacccttgccctgcatatatacgagcagatttcacaatatcatacttaacagtactaagcaatgtttttatagcggtcgtaacaattctaccgctattgataatctcaacagaaaaccaattatacaggctacccgcatgaaacacaacttgtgaagatgatcttaaatccgttttgaagatgacctccattttcatggatatatttaaaataaaatccattcaattttaaaattataaaataataagaagatgccctctaatatgaaacagttttgcaagatttctgtatggctacagcagcacgatccagatttattagaaattatcaacaacttatgtatgcttggcaatttatccgcggcaaagtacaaacacggagttaccttcatttaccccaaacaggcaaagatccgcgatgaaataaaaaaacatgcctactccaatgacccttcacaagccataaagaccttagaatcactcatccttccattttacattcccactccagcggagttcaccggggaaatcggctcctacaccggagtgaaattagaggttgaaaaaacggaggcgaataaagttattttaaaaaatggagaagcggtcctagtaccggcggccgattttaagccctttcctgatcgccgactagcggtctggatcatggagtcaggctctatgcccctggagggtcccccctataagcggaaaaaggagggtggggggaatgacccgccggttcctaagcatatctcgccgtatactccgcgcacgcgtattgccattgaggtggaaaaggcctttgatgactgtatgcgtcaaaactggtgtagtgtcaataatccctatcttgccaagtcggtctccttgctgtctttcttgtcgctcaaccatcccaccgagtttattaaggtactgccgcttatagactttgaccccttggtgaccttttatctacttcttgagccctataaaacgcatggggatgactttttaattccggaaaccattttattcggccctaccggatggaatggtacagatctgtatcaaagtgccatgctggagtttaaaaagttttttacccagattactcgccaaacctttatggacatagccgattcggctactaaggaggtagatgttcccatatgttactcggatcccgaaaccgtacattcctatgccaatcacgtgcgtactgaaattttgcatcacaatgccgtcaataaggttacaacacctaacctcgtcgtgcaggcctataatgagctcgagcaaaccaataccatacgacattacggccctattttcccggaaagtaccatcaacgcactgcgtttttggaaaaagctgtggcaggatgaacagcgatttgttatccacggcctgcaccgcacgttgatggatcaacccacctatgaaacctctgagtttgcagagatcgttagaaatttacggttttcgcgtcccggcaataactatataaacgagcttaatattacaagtcccgctatgtacggcgacaagcataccaccggagatattgcgcccaatgatagatttgccatgttggtggcctttatcaacagtactgactttttatacaccgcgattcccgaggaaaaggtaggggggaatgaaacccaaaccagtagccttacagacctagttccaacacggctacactcttttttaaatcataatctaagcaaacttaaaatcttaaaccgcgcgcagcaaacggttagaaatattctttcaaatgattgtcttaatcaactgaaacattatgttaaacacacgggaaaaaatgaaatactaaagttacttcaagaataagtatgttgatacctgtggtgtgttttacctgtgggtttcctattggaacctacgcggcaatttttgacaaggctcgtaccgagtatattaaaaccaaaatgggcggaacattgccgcaaaatatcccattagatgcttctctccagattgagttaaaagacctcattacagctctgggaatcccaatgcgggtgtgttgtcgcactcatttaattactacgttggattatcgtaaatattattaatatctaaaattgaaaaaatatttttaatgttactagtaaaaatgactacacacatctttcacgcagatgatctcctacaagcattgcaacaagcaaaagcagaaaaaaatttttcatctgtattttctttagattgggataaattacgcacagcgaagcgtaatacaacggttaaatatgttacggtcaatgtcatagtaaaaggcaaaaaagctccgctaatgtttaactttcaaaatgaaaaacatgtaggaaccattcctcccagtaccgatgaagaggttatacggatgaatgctgaaaatccaaagtttttggtgaaaaaacgtgacagggatccctgtttgcagttcaacaaatacaaaatctcgccgccattggaagatgatggtctcactgttaaaaagaatgagcagggtgaagaaatataccccggcgacgaagaaaaatctaagttgtttcaaattattgaactgttagaagaagcctttgaagacgctgtgcaaaaaggtcctgaagccatgaaaacgaaacatgttataaaattaattcaaagaaaaatttctaatagcgcggttaaaaacgcagacaaacctttgccgaatcctatcgcacgcattcgtattaaaatcaatcccgctacaagtatactaacaccaatattgcttgataaaaataagcccattactttacagaatggtaaaacaagctttgaagagttaaaagatgaagacggcgttaaggccaatccggataatattcataagcttatagaatcgcattctatacatgatggcatcattaatgctagatctatttgcatcagcaatatgggcatttcatttccgctttgcttggaaatgggagttgtaaaagtttttgaaaaaaataatgggattgatgtgaactccatttatggctcagacgatatttcaactcttgttaatcagattgctattgcttaaacaatttgctcaaaacaagcttataaacgtttcttaggtatgcgatacgtaaatcctaattctttaataagttctttttcagtagtgatttttagaggtactaaagtttgatttttaaataatccatactgatttagcttataattctttttttttaacgcagctcgaattcttattaaataagaaacgggacccgtaaaatgaagtactgcgtatggcttttcctcggctaaggccgtaaaaagatcaagttgatatgtgttttttttccattcaataaaaagtacacactttcgttctccgcagacttttacagaaaaagaaagatcctttatgcgaatgttgggcaggacgtgttttaaaagttttttttctggaacaataataagaagatccacgtcattaagcattttctcttcgcgtcttaagctaccaacagcaacgatgttttttgataaaatttttataagttgtccattatattcaaacgcaagtcgggagcgtaagtcatttacaattttttttccttgaataagcgttaacattttatatttaatattaaaatcttttcattttatatattatatacgcaaaatggcacttgatggttcaagtggtggaggctctaatgtagaaacattacttatagtagcaatcattgtggttattatggcaatcatgctttactatttttggtggatgccccgccagcaaaaaaaatgtagcaaggctgaagaatgcacatgtaataacggaagctgttccctaaaaacaagttaaaacatgcaattatatgcatgcatataaacgcatgcatataaacgcatacatataaaatgcgtaaatactatataaaaaactataacatatcaatcaaggaatcaacacttttataattttccgtaatatatttttcatccataatgatgtcagagtacatggtccctatgcgaggaacagagcccataagggtaggcgcggcaataccgtaaatgggattcacggcggagtcaaccgcagcatctgtcaagacctggactggagacgacaaggccattcgcaacaacacgttggaaggctctcttgcattaagccctgccttttctagagaggtaacctgtcccgttcttgtcatgagatctgcgtacatgagtaaatgacgatggttgggacccttgtcccccataaccgttctaatttcactaataattttttgccgtgccgcttctatgccgtaaagctccatggtgtctcctatagaggacgatacgatggtgtatgggtcgatgttatcatcaagcattgcgccaaaaatattagtcccgtttgttttgatggcgtagatattgtctagtcttaccagtttcccctgggcatccacacggtggcgcataagcttaacaacattcgcatttttgatgcctggtattcctctaatcgtgctatttaatagtttatccaccacatttacggcaattttttcatccgtagccattcgggtattggtactgcgtctaaaggcgctttcccgtaggtatatgcgaataatgatgggaatccctgaggccgtgttttccacagaatgcatgatgtaggtgttggggtgtttagctcttagactattaataatactttctagactaatgctttttaatatcatggttgttttgtttaattccaagcggatacaccagtttgcaatatcctctgggggctgtagtagaggatggttttccagaaaatccgtcatccattccacatcacttgcaaaatcggggtacatcacatttttttttgtgcttgaatacgtttcgtacaataggtgccactgcaatatcaaccgttcgaacgttataagctctatgctgttagcaatttcttgcgcatatgttttatttgtttccacttccgggttctttagacgtaaaagcatttcagaggattgttcagcctctacgggcttcgcgctaaagatctcctggggccgcacaattcccgacttgttggttcccccggccacggaccggtggtgggagtccagcatatattgtgtcaagggctctgatacggactgcgccgccaggattcccactgcctcaccgtagttaataagactttgagtatattgtagccttatgaggtccaggatggcactcatctgctcgcaggtaatgtttaatgttttaacggttgccagttcgatgcgaataagcatgcgcatcagagaggcagcccgtttaagataaacgggtatgggcgtttgtagtcgttcctgaatgttgttaataaacacgtatggaagatttttgcaaaacgttttgaccatcgcgtatttttgtagaatacttttttcgtcgaagggaagcacgccactggtggagctcagtagaatgttttttacgatgctggccacgtttaccggcacctgtctaacatctgtaagcagctgactgaaattaaaattttcgacgtttaggaagatctgtcgatatttatctctatcctttttaaggcgtgaaaattcttcttcaaacaagggcgattgtatcccggtgtacttgaatttgtcttcaagttcctggtccgacagcatgatggtttcaaaccgtacggtttcaagctggcgcgcatcaaggccgtcctctccgtacaactgctgcacaagacgcgtatcgatggaaacccgtcggtaataatccacaatacaggattgaaggccaaagatggctttacggttggcatagcctgtggatgatgtcgataatgctttgttgatcaagtcgaatcttccattcatttccccaaagataaattcaggggaggtaaggcccgcaatatagctgttgcagatgaacccgtaggcctgcgcctccagggcaaacctggggtagtacaccagggtcctaccgaaggaaaactggggttgaatgcgttgtgtattaatttcaatttggccgatgcccgccatgatgtgaatcatattggggtttgagcccttggcgccagtggccaccatctgaaaaagcccattggtttccggattaatggaattcataatcggctttaaaattctatcgggaaatttaagcgcattcagctgcaatttttcgtagaagtcatgcgttgtcaggcctataggcggcatgatgtctccatgaagcagccggttgtttatttcctccgactcaagcagcagttcattgataatttcttggacctcctgatgtgcctccggggttaggagcatgtcggccgtggacactgtgaatccggcgttgcgcacgtagtttagggcgagctgctgggtcgcaaatatcattttcaaggcctgctgcggcccatacctacgcgaaataaggtgatagattccaccggaggaacccgctccgacggcctttttgtcaaggacgccttcaatgagttcgccgttgcgtatttgtgtagagatgtcctgcttgttataatgcatgtagggtgcatacacttctgagtaccatgtgggggctcgttgataattgatgggggtctgcctcagtagcatagatacaaccgatttgccatccagcaggtcagttggggagtagttggcaaaacaaggtgggtcggtttgggttgtttgaaacaaccccatggcgtgcagcttgttcatcacatttttccccatgggggtgttcgtgcgtgtaagcaaaaagcttcccaccgtggagtcctgcacctgcccattaacgggacccgagctctttgtggaaatgaaccagtttcgcacagaacaaagtagttcggcctcaacgcggctcatgacgctccagggaacccagagattcatctgatccccgtcaaagtccgcattataccaggcacatgcgctgacattcatttgaaacgtagaaatttttgggttttcaagaacgacaatccggtgaacccctatgctgcttcgttcgagagaaggctggcgattaaaaaacgcgacgtcgccagtgacgacgtcacggtaaaggatgtctcctacctccagcctaaagtcttgtttgagaccctcaatgtcgtgaacggattgtgttatttgcttatacactcttgaacaaccagggtactggcgctttccatttaaaaaatagggcattaatctattaatattataatgttgcactgtttccgcaacttgcagcgttcgtgcaaaggaaatgggatagccaacctcgtccaggtgaaggtctgagttcccgcagatggtggaccggctgatcgaccatacctggctgcccagtagggatttacgaattcttccctccttgcgaggaagtcttcgcatgatggagggagcagggcgtgcccccatgacgatcccacgctttcccgtgcctccctgggttgcggtggtggaaacggaatccaacaaaaagttatagtaaagttgctgtatggtttgcaaattgcggtcaatatttaaaggtattttttggccgcgcacgatttgtaggtccttcgggatcagcagattctttcgaaccagatactgaatcacgttgttaatgtcgtgaaagctttgggggcctgacccgattcccaatctgatgccaggtcgtatgctgatgggggggatctgaatggccttaagcacaagtttttcgggatgggagtttttacttcgccccagttttacaacggtgtcgtaggttacgcgcgaaaaaatctctctgatgatctgcgggtacagtttgtcaatcttgccctgctgatccgcccaaaaggtaaaataatcttccgagtccttaacaattttggggtgtactgccttacagacgtagcactgctttccttcggtttggcttgaagccgcttcaataagacgcttaggcctaataaggtgctcgtacctctttaggtcaacgatgggagccccgcagttgagacatataacccttaaccatcgtcgtatttcggcgatgaagagcggctgaagcaccggagcatgcatctgcagtatcccagggtgtcccatacattgcttgcgctggtgtgagcaagtgatgcatttataatggtgatcggtggttcccattcgcgcatcatagatacccccttcggcgggaagggtgccctcaaataaattagaaatggtaacctccataacgccttgcctcttatgatcattgtcaccggcaatattgaactgaacggcggctatttcggcatatccagcctccatatttttgctaaatacataataaaacttcaaatgttaaaaaaaaataacatcggttggcatatttttttgttaaaaccaagtgttaaatgatttctaaaacacttatcggttcacgaaaacctaccgcacgggcctgaagaggaatgccagttttgggggaaagctcggcatattccacggtaagctcttttccataaagatgttttttaaataaggcgggcgtgagtttttgaaaaagagcataacgatccgcgtacgtcaaatgcttaggagtgactacaaaccgctttttgtttggcaattcgcaaacccataaaatggcgcctaagtcctttcccttttttccctgagtatagtccactaaaataaattcagcgtctagcagcggtttcagcttggcaagatgcgctgagtggtagttgttgtatcccggctcatagggcccattggcattgcgtacgatggctccctcgtagccctccttaataaactgcgccttaagcctaagggcctcatccacattcttcacgctaaaattttcaacttggtggataaaggtaagatcttccttctgtttaaaaatatttgttaatagctgttgtctcttgttggaaggcatttgaagctgatcactccaaaaacagtcaaacacgtaaaagtgcagctcggaggaatctgtcttcgcattcgcctgccccgcgatccattgcagaggtttgcggtgtaaataaagctcaccatccaaatatactctcacgtctataaataaataaagctgtttgagctcttttttaatattgtcaagacctaaaaattcctttttcgtgcgcgaatacaagagaatgctaccatcgccctgctggcaggccacagctcgaacgccattacgcttgcgctgcacgatgggatctgtttcttcttcaaaaaatgtcttaggaattatattaaaatattttaccagcataggggggataattcctctatttgtgtgggctccccgcttttgtctggcatggcgattatatttactaagggcgtccttgaatgcctgatggactaccgttgtggcattttttttacccaagttttttccctcggtaacacgtgtcatttttgatatccgcaccgccccttcttccacaaaaaattttgtgaaaatttcagcaacggcgtcttttacatctgtggaaaacatctcatctgtgatgggaatgatcgtgttgtgctgcaccacttgcacacaaataatccatgaggccttttttccgcttttcgtttcagactcaatcggaggaaaacaaaaaatgttgtttgaatattgcccaggaaattgatttagcatggttttaacaataaaataagcctatcaatttttttataatttgaatagttattccaaattcaatatggcttctttagataatttagtggcacgatatcagaggtgctttaatgaccagtctcttaaaaatagtactattgaacttgaaatacgttttcaacagataaattttttattattcaaaaccgtatatgaggcacttgtggcacaagagatccctagcaccatctcccacagcatccgctgcatcaaaaaagttcaccatgaaaaccactgccgggaaaaaattttgccgtcggaaaatctttacttcaaaaaacagcctctcatgttttttaagttttcagagcctgcatctctgggctgtaaggtctcgctggccatcgagcagcccattcgtaaatttatcttggactcctccattctcgttcggctcaaaaatcgtacgacctttcgggtatctgaactttggaaaatagagcttaccattgtaaagcagctgatgggaagcgaggtctctgcaaaacttgccgctttcaaaacgcttctgtttgacaccccagagcaacaaacgacaaaaaatatgatgacgttaataaacccagatgacgaatatctttacgaaatagaaatagagtatacaggaaagcccgaatccctaacggcggcagatgttataaaaattaaaaacacggtgttgacacttatttctccaaaccatttaatgctaacagcctaccaccaggccattgaattcattgcctcccatatactgtcctcagaaatccttcttgctcgtattaagagcgggaagtgggggcttaaacgcctcctcccccaggtgaaatccatgaccaaagcggattacatgaaattttatccgcccgttggctactatgtaacggacaaagcagatggaattagaggcatcgccgtcattcaggacacgcaaatttatgtggttgcagaccagttatacagcctaggtaccaccggcattgaaccccttaaaccaaccattttggacggtgaatttatgcctgaaaaaaaagaattttatgggtttgacgtcatcatgtatgagggcaatctattgacgcaacaggggtttgaaacaagaattgagtctttaagcaagggcattaaagtcttacaagcgtttaacataaaagcagaaatgaagccctttatttcgctaacaagtgcagatcccaacgtgctcctcaaaaactttgaaagcatttttaagaaaaaaactcgcccatattctattgatggcatcattttagtagaacctggcaattcttatctaaatacaaacacctttaagtggaagcccacctgggataacacattagactttttggtgcgaaaatgtccggagagtttaaacgtaccagagtacgcgcccaaaaaagggttttccctgcatctactatttgtaggcatctccggagagctttttaaaaaattagcgctaaattggtgtccaggatatacgaaactattccccgttacacagcgcaaccaaaactactttccagtacagttccagccatcggattttccattggcatttctttattaccacccagatacctcgtcattttctaatatagatggaaaggtccttgaaatgcgttgtcttaagagagaaatcaatcacgtcagctgggaaattgtaaaaatccgggaggataggcagcaggatcttaaaaccggcgggtattttggcaatgatttcaaaacagccgaactcacatggcttaactatatggatcccttttcctttgaggagctggcaaagggcccttctggaatgtacttcgccggtgccaaaaccggcatataccgcgctcaaacagcacttatttcctttattaaacaagaaatcatccaaaaaataagtcaccaatcctgggttatcgatcttggaataggaaaagggcaggacctaggacgttacctggacgcagggataaggcatcttgttgggatcgataaggatcaaaccgcgcttgcggagcttgtttatcgaaaattttcgcatgctacgacccgacagcacaagcacgctaccaacatttacgtgttgcatcaagacctcgcagagcctgcgaaagaaatcagcgaaaaggtacaccaaatttacgggtttcccaaggagggagcttcttccattgttagcaacctgtttattcactatcttatgaaaaacacgcagcaggtggaaaacctggccgttctgtgccataagcttcttcagccggggggaatggtgtggtttaccaccatgttgggagaacaggtcttagaattacttcatgaaaatagaatagagctcaatgaagtatgggaggctcgtgaaaacgaagtggtcaaatttgctattaaacgtctctttaaagaggatatattacaggaaactgggcaagaaattggagtcctgttacccttcagcaatggcgacttctacaatgaatatcttgtgaacacagcgtttttaattaaaatatttaaacatcacggcttttccctagttcaaaagcagtcctttaaggactggattccagaatttcaaaactttagtaaaagtttgtataaaattcttacagaagccgataaaacttggacaagcctttttgggtttatttgtctgcgcaaaaattaaatattttttcataagaagtactacccaggttttaaagaaatagctaaaaatatcatatggatactgccatgcagcttaaaacgtctattggtttaattacatgtcgtatgaacacccaaaataaccaaatagaaactattctggttcaaaaacgttacagccttgctttttcagaatttattcattgtcattactctataaatgctaatcaaggtcatctgattaaaatgtttaataacatgacaattaatgaacgactgcttgtcaaaacactggattttgaccgcatgtggtatcatatttggattgaaactccagtctacgaactataccacaaaaaataccaaaaatttaggaaaaattggcttctcccggataatgggaaaaagcttatttcattaatcaaccaagcaaagggctcaggaacacttctatgggaaatccctaagggtaagccgaaggaagacgagtcggaccttacctgtgccatacgggagtttgaagaagaaaccgggattacccgcgaatattaccagattctcccagagtttaaaaaatctatgtcatactttgacggtaaaacagaatataagcatatctacttccttgcaatgttatgtaagtcgttggaggaacccaatatgaatctttctttacaatacgaaaaccgaattgccgaaatttctaaaatttcttggcaaaatatggaggctgtacgttttattagcaaacgccagtcattaaacctggagcctatcatcgggcctgcatttaattttattaaaaactatttacgatacaagcactaggatgccgcattaaaatgccacataaggtaatacactaggaatgtcgcacacgcacaagaatacaacgtcgccggagatttattatctagtacacgttttatgtatgtacaatccgccttcatttaatatattgagcggatgtactatgtatttattttaacaaaaaacattatttttttttaatcttcatcatctgtttttataaactcagtaatatcaaaagtagcttgtggggtttcagagggttcaccttggttatcctccgtgaggataacatgttcttcaggttcgtcgtcactggagaacccatcatttaattcctcttcactcaacatctgtaaaaaatcttccaagctttcgctatcgttaaaatcctcatcatccataagaataatggtaccttcctcatcgtttcctccttgtttcgtgtctaaataggcctgcatggcatttgcaaaagtatcaaaataggctgagtcagattgctgttccaaaatatggccttgcgtattaaatgtggttgcatcgttgttaaatgcttgcaaatacagtaagggatttatatccattattattaagcaaaaaaaatttaaattatttttcgaccgatgttaggtaaaattaaacaattgctataggtgttaagcaatgtttattgattttaagtactcaacaaccatgatgtaaatactatacagcacttttggatttttaatcaaatccagattaatactaacttcttttgtgatacagttcgtaataatagtatcctgctcatcgttttgtaagatttcttttaatatatttttttttaccgggatactaagcaattgattattttcttttaaaaactccttttgatattcaatcgtcttattcattgaatatttgtatataactataattacaaatgttcaatgaattgttattcatgtcgggagatggctatttaaaaatcatgtcctatttttctttgctcaataagcatccaaatattttcatggcgttttattaattgttcattattgaacgtatcacaaagatcatttataaattgcagatagtttattatttctttcaagagagtaacaaacattacttcagcagaacatataataggtaattcagtggcgttaaaagaattttgatcttgttgatacgccaatggcgaggacttaaggagatttgggggtcttgcccaaaaccctaggctgctgttcttgttttttagggcgtcataaagaaatgaaagcacattgcaaggcttaagccgcgacatctccttccccttgggccctttccatatttttagatctaagatctcatccgagcttatagagtaggtatagtaaagtttttcaaaaaagcatatctgcttgaagtcttttttagaacgactttcaagaagcatttctataatgttaacaagttttgttaggtttaaggcctgttcctgtgtaagctcctcttgcacgtgatagactgaaaaagtgtgcttaggaatgaaaatactccccgtggcactggcctgttgtctgccaggtatatagtacacgctgctgttagcaagctgtaccggcacaatttgccccacttctgcaacattattttgcgattcggacgagggtatgacaatagttacgggttcagtcaataggctttcgccgagaataatattactgtcatttttaataattttaacggccgctattaaatcaaaggcatttaagtaagaaacaacagcagaaaatcttacatgcatatatcctcttccgctattattcgtacgcataataaaacaaggggagcgttgtataacgccagtaatattaagaataaaactgtttttgaaacacttacccacataaatgttttcaagctccttcaaaagatgagcctccacatttgtacaaaaattggtaggatcatcaatattcaacgttgtctcaaaaattttttggtcgatcatatctataatatattctgtctatttcaatttaaataatatacgaataaataacgagattattttattaaataagcaatggtgtatacactttgtatttactttgagatatactttgtgtatcacaacgtgccctaagatgtgtgcacaagtgacggcattttgtcgttaaaaaggtaaaaccagcggattccatcctgcattccatttggttgattacgagcctccatttctttttgcaaaaggttattgcgaatgagtaagcagagcttgatggcactaatctttgtaaggtttaaacttatgcccaattggtcagcaattttttgttgctcctcccgtccgcgtgtttcgcatacggctccccggtttagcatgcgaatatcagtaatctcattcttttttaaaacctggataggtgggcggattttaaatttaagggcctttcccttgctttccatatagcctatgacgatgtcgttttcttttcgtttaacattaatattaagcatataaagcggaatttcatgccaggttttatcttctcgcgaggtaataagtcgcacggagtcctccgtggcatagcccactagagtgttgtcatccccaggcacgtggcttataattttaaaaatgtccggaaatggctgaatatctttttttgaaaaagcgatgaaaaactttttataaacctcgacaagggcccccatacctgcaagattatctataataagtgcttctagcatcgtatagtgaaatgaagcggggtagtggatgagtacctgctccattggctcatcctgaaaatccttctgaaacttttcatacaatacttgaaagggttctttggtctgcgagtgttcgaggtatttggtaatacggatgctgtgcatcgcgggaggctgaaaatcccgaatatatgtttcaatatctaataccggttcctttttatggttaagcaccgcagcgacgtacaaatgctcaggctttgccggcacatgcataatggtgcaaagacgattctgtatccataattccttgcactggttttttgagtagcatagagaaatgagcgccagcgcgaagttgtcctctgagaagagtttattatcgatggtaattccctgtatgagcttgggagtggaaacagccttccatagctcggagtacgtccacacggggcgtgccataaacaaagatataataatattagaaattgtttttacctcttgctccccgtatccataggcctcaaaggtattgaggacggtggctccgacgtttgccggcgtgatggatggactaaggggcagactttccaacataggcttatcaatcttaatctggttggtgaacccatcaatggcgtgctttcgcagcgccttatccccctcctgtattaaaatgtattcttttaatttttgtgcgtacttagcgagctctggccctccatcgggtgttgtcgatacgtacaaataaattgtcacgttgcgctcactgggggggagctccatgtgtgaattttttcgcaccaccctcccaaatacctgaataagccggggaatatcaaggggcaatgacataatcatctcgtaccgcacggcctgaaagttcaaaccctccacaatcaccttggacccgatgagaatacgcagctggtggccttccaggttggacgaggcgttaaaaagagccaggcttcgttcgcgtacagcgggctctatttcgctgtgcagaatggtgaaccgtactggaataaactgatggtcgctatgtgtgtgctcatcgcgaatcgcggcgcagatggagcagcgggtcgttcccacaggggacgaaacttcatttaaaatgccattactttgtaaaatttcttgcaagataagaacccccgacatgcggacccgattgtggtaaattaaaattttcccccggccttgccgaataatggaaagaatgtctttcatcatttgagtgtattttccgctataaaaggccaatcccgagatgtgcgttggtggctgcagcgacaaaaagctgccactcacattaaagggggctctacgcgaaggctcaataatctgtaccccgttttccagaagccagtctgtgcttgccatagaaagggcggtgggggtttccgtcgagttaaacaggccgtaagccttgggttccgtttgttttgaaaattttgggttgggaaacaccatgtcataaatgctgtacgcattactcgagattttagggtcagggcccagctgtttaagcgtttcaagctgatactcagacatggggcattcgatgaaatgtaagtacggcaatgtttcgtctttataggacaacatctttccggcaaatattctttcggggtaaaaattggtgttggtatccaacaaaaaagatacccttccggtgctcagtctttccacaagagctagggcgtcctttttccatttaacggaatgcccactgctgtcaaacagttgctggcgctggaggggctggccgttgggcagctcatgccgcggaaccaaaaggtttaacaggtcgacgtattccatgacactcccggttacgggcgttgccgacatgaagacggccctgggggcctggtgaggtggaaaggcatccaggacatactgtaaagcgatgccataattatttcgttcctggatattgtacacgttgtgtatttcatccgcaatgagcagtcctcccctaagttgctccatgattttttgattcacccggatgaggccgtttgtctcggcctcgctaattttttgcacgaactgagatatatcgttctcattcaatgtatcttctgcttcgtcagaacgatgaaacagagaaagcacatcaaagtttttctcttcacccttactcgtaatattgaaaagcttggatgcaaattccttatagccgtaaaactgaaaaaagcctccgcggtttctatcggttaaacggcgctttaacgtactaacgaacccatttagatgccgtgattcgaccgacgtggtgctgccagactgctttgcaatgtgaagaagccggtgtagctcagcgacctccttgtaagaaacaaatcccagctcaggacgtcttagcatttctgtttgaatgatggcgcgtgtaaagcctaccacaaaaatccagggcgcattttcaataaaattcatgtagtggttcataaattgacgcgcgatggcaatcgcggcaatgctttttcccgtcccggtctgccagtttaataaaagacgcgagtagggcgtgttgggattttgaaagttttggacgaaaagctgggcattatgcaattggagacccttgatggaaggaaagggcgacgcgtaggggtcacacggaaaaaacgctcgcccccccttctcgcagccaggcccaccgatctggacaaaatgagcccgcagatcacgaatgagctctttttggtcgacaggaggggaaatcaacgatttaaactcctttcttcgcgccaactgctgcaaaaagtctgcggcatccaattcgggatacgccatattatcataaaaaaaataaacctttttatgaaaacttttatgtgattctgtattgcaattgttttttatgaatactgtaaataagcgtatcaacttgtttttctaacgaagaggcgttattctttttttctggatataaaataataataagtataataattaagactaaacagcaggcaatcactatcaaactcatattatacttacttttttataaaaagtattatatcttatgaatgcgcaagttcagctaattgttcgtcgcttggaatgtgggactgcagggaggtggagtttttcctttttctaaagaataccgggaaatggtggtgaggctcaggttgttgtacatagtagctaggaggaggtttaggtatgctcgacttgcagtcaatagtccggttatagtaaacgatggcaacgatgataagaataataatgagcaaaatcaaaatgcccaggagaatcgcagttgttccgggatatttggcgattgtatgggctaaaaggccttgggtgctttgtttaattccctcgcgggttgacaggttatgagaaagcagtggagacgtttcagtgtccatttattacaattgaacagttatattaatctcaaataaaatataacacaaaattaattatggccatgcaaaagttatttacgtatatttacgagtttattgaatatcgtaagatggtgctgttggaagaaaaggtaccatatgataagtttgttcaaatggtacttaatacaggattttttcgtattaacgcggagacgctgaatcacggaatcgtatccgtgtttatctttggagcaaatggcaagtacgttcaccacggaggcgacatgagaacgcttttaacgaatacgcttaatgaaaaaaaacattatgaagaattaattttaatcgttgataagcccgttttaagcaaaaaaaatattttagatataatcgtcgagcagcgcgctgcaaatcccacgattgtaataaacatatatccctaccacctgttctgcattaacattcccaaggtgagtgccattcctaaacataaactaattactcaggaggaggcgcaggagtttttaggtcgcgaatatctgcaaccgcaggacctcatgcaaattagcgcgtcagaccccccggtggtctggctgggaggaagaccgggagactttgtgcaaattgagcggccctcagagacagctatgcacgctgttgttatccgctttatcaccaagtccaaaatttgagtcccgtgtttaaagatgacagacagctaagtaagcatatctgtaaaattgtcgatgtcctctgtggatagagcgctttcctctgagcagcaaattttttcatacatctccatgggggatggcgaggctttaatagtatgtaggtcacgtaagaactgttgtatgatgggatatttgtcttttaaaaactggggatgtttcataactggaattatttgaaagataaagaccttccatccaaagtagccaaccacatttggcatttcgggacacgcggtttcataaggcatagaatagtgaatagtgtactgatctttttgatacagcgtttcaagtagttggcgaaatgtttccgcgtcgagcgtgccaaaatcttgaggagcctcggtgtgctcctgtgtagagcagatcgtgatgattccccaggcaagcgggagcatggactctggagggtggatatccgtattggtctcattattcgatcccagctgatgaatgccgcacacgcgaaacatggcctcgacgtagatgcccatagagataggcggcgaaagggcaagaccggattgtatttgcggcatatagtaggagggcaccgagttttttatttttcggttgaatggggactttatttctaccagcacggggatgcgtttcgtggcctcatagcgtacgttgttaaaaattgttttgatttcccaggactgttgagtgtatcccagcgttaggtgacaaaacccatcggggctattactatgtccggggtatcccaaataggtcccatcaatatgaatattgtcacctatgacggtggtttggcagaacaactcaagcagatctttactaacacgctcaaaaagggttccccagctacaagcagcgcggttcaaattcttcttaaaaagatttgctttttccgccaaggttatataatagcttttgtaagggtttaaacctaaaacgctggcaaggtcagagccacccacctgagtgcgacgaatagcatgccaggcatcggagcgctgctgaggagagtctttaaacaggcgtacaaaggtttccattatacttgttttaacaggaattcaatataaaaagtcaacacagtttgcaatttttccaatctcaagatatagccatacatttttttttccaattggcgaatatgtttaagctcatgtgtttcaatattagcatccggaaatttaaatgcataaagatgttcaaaggcctgatttatacacgtatcaaaggatctgtggtatgttattagcttcagcatgtgtgccagatcttcaagatggtctaaatttatacggttttccacgtggtggatcatgtctgccacatcttgagcccccatccaggggatcacaaggtactcccccttaaagatgattcgtcgtttttttaaaaaatcatgaaaacgttttaaagcttcaagaaaggggcagttgggctttgaccccaaaatgctgacgacgatatcctcgggcatgatgtattcgcagtgaggatagtagtttacggactctaattcagcggcccgccgttttatttcgtatcttgcccagttattcagagagtactccacgcctccgaccacaacagacatcctatctattaaaaaataacaataaaaaccttatgaaatctatgtatagtggccgctaaaatgtctatattagaaaaaattacgtcaagtccctctgaatgcgcagagcatcttacaaacaaagatagctgtttaagtaaaaaaatacaaaaagagctcacctcttttttggaaaaaaaagagacactcggttgcgattcggagtcctgcgtaattacccaccccgccgtgaaggcctatgcgcaacaaaagggactggacctctccaaagaactggagactcggtttaaagcgccaggacccagaaacaacacgggtcttcttacaaacttcaatattgatgaaacgctgcagaggtgggccataaaatacaccaagtttttcaactgtcctttttccatgatggactttgagagggtccattataaatttaatcaagtggatatggtaaaggtatataagggagaagagctacaatatgtagaaggcaaagtggtcaagcgtccttgtaacaccttcggatgcgttttaaacacggacttttcaacgggcactggaaaacactgggtagccatctttgtggatatgcggggcgactgctggagcatcgaatattttaattcgacgggaaattctcctccaggtcccgttattcgttggatggaacgggtcaaacagcagctattaaaaatacaccacaccgtgaaaacgcttgcagttaccaacattcgtcaccaacggtcgcagaccgagtgcggcccctacagcctgttttacatcagggcacgcctcgacaacgtgtcatacgcccattttatatccgctaggattaccgacgaagacatgtataagtttagaacccatctgtttcgcatcgcataaactaataaagtttgaattctttataggaataaaaatggaagcgtttgaaatcagcgatttcaaagagcatgcgaagaaaaaaagcatgtgggctggcgccctcaacaaagtcactatttcgggtcttatgggggtctttaccgaagatgaggaccttatggcgttacccattcacagagaccactgccccgctttgttaaaaatttttgacgagatcatcgtaaatgccacggatcatgaaagagcttgccataacaaaacaaaaaaggtaacttacattaaaatttcgtttgataaaggtgtgttttcttgcgaaaacgatggcccgggaatccccattgcaaagcatgagcaagccagtcttatcgccaagcgcgatgtgtatgttcccgaggtggcttcatgtcactttttagccggaacgaacatcaataaggccaaggactgtatcaaggggggaaccaacggcgtcgggctgaagctcgccatggtgcattcgcagtgggccattcttaccaccgccgacggcgcgcaaaagtatgttcaacatatcaaccaacgcctagatatcattgagcctcctaccattacaccctccagggaaatgtttacacgtatcgagctcatgcccgtataccaggaactagggtacgcggagcctctgtctgaaacagagcaggcggatctttccgcctggatttaccttcgcgcctgccaatgcgcggcctacgtgggaaaaggcaccaccatttattacaatgataagccttgccgcacgggctctgtgatggcgctagccaaaatgtacaccctgttgagcgcgcctaatagcacgatacatacggcgaccattaaggccgacgcaaagccctatagcctgcaccccctgcaggttgcggcggtcgtgtcccccaagtttaaaaaatttgaacacgtgtccgttatcaacggggtaaattgcgtaaaaggagaacatgtcacctttttgaaaaagactattaatgaaatggtcgttaaaaaatttcaacaaacgattaaagataaaaaccgcaaaacaacattacgagacagctgttcaaacatctttatcgttatagtgggttccattccaggaatagaatggaccggccagcggaaggatgaacttagcatcgcggaaaatgtttttaaaacgcattactccattccttctagttttttaacaagtatgacaaagtctatcgtggatattcttctgcaatccatttctaaaaaagataaccataaacaggtcgacgtagacaaatatacgcgtgcccgcaatgcgggaggaaaaagggcgcaggactgcatgctactcgcggcggaaggggatagcgcactttccctgctgcgcacgggactaaccctgggaaagtccaacccaagcgggccctcctttgacttctgcggcatgatctccctgggaggagtcatcatgaatgcctgcaaaaaggtgacaaacattacaacggactctggagaaaccattatggtgcgcaacgaacagcttaccaataataaagtgttgcagggaatcgtgcaggtattgggtctagacttcaactgccattacaaaacacaggaagagcgagcaaagctgagatacggctgcattgttgcgtgcgttgatcaagatctggatgggtgtggaaaaatccttggactgctgctggcctactttcacctgttttggcctcagcttattatccatggtttcgtaaaacgactgcttaccccgctgatacgtgtgtatgaaaagggtaagaccatgcccgtggaattttactatgaacaagagtttgatgcctgggcaaaaaagcagaccagcttagccaaccataccgtaaaatattacaagggattggcggcgcatgacacccatgaagtaaaaagcatgttcaaacattttgacaacatggtgtacacgtttaccctggatgactcagcaaaggagttgtttcatatttattttggcggggagtcggagttgcgaaaaagagagctttgcaccggcgtggtgccgctcaccgaaacccagacgcagtccattcatagtgtccgacgaattccttgcagcctgcatctgcaagtagataccaaggcttacaagctggatgccatcgagcggcagattcccaacttcttagacgggatgacgcgggcgcggcgcaaaattttagccgggggggtgaaatgcttcgcctccaacaaccgtgaacgaaaggtttttcagttcgggggctacgttgcagatcacatgttttatcaccatggcgacatgtcgttaaacacaagtattataaaagccgcccagtattacccaggctcctcccacctctatccggtattcataggcataggaagttttggctccaggcacctgggaggaaaggatgcaggatccccaagatacatcagtgtgcagcttgcgtctgaatttattaaaacaatgttccccgcggaggactcatggcttctcccctacgtctttgaggacggccagcgggcggaaccagagtactacgtgcctgtgttgccgcttgctattatggagtacggcgccaacccatcggagggctggaagtacaccacttgggcccggcaactggaagacattttggccttggtgagggcctacgtcgacaaagacaacccaaaacacgagctactgcactatgcaataaaacataagattactatactcccgctgcggccctccaattacaatttcaagggccatttgaagcggtttggccaatactactacagctacggcacgtacgtcatctcagagcagcgaaatataattactattacggagcttcctctgcgtgttcctacggttgcatacatcgaaagtataaaaaaatcgagtaaccgcatgacatttattgaagaaatcatcgactacagtagttcagaaactattgaaattctggtgaaattaaagccaaatagtcttaaccgtatcgtggaagaatttaaggagactgaagagcaagattccatagaaaattttctgcgcctgcgcaattgtttacattcacatctaaactttgtaaaacctaaaggtggcattatcgagtttaacacgtattatgaaattttgtatgcgtggctaccttacaggcgtgagctttaccaaaagcgtcttatgcgtgagcacgcggtgcttaagctgcgcattatcatggaaactgctattgtacgctacatcaatgagtctgcagagctaaatctttcccattatgaggatgaaaaggaggcaagccgcattctaagcgagcatggatttcccccgctgaaccacacgctgatcatttcccctgagtttgcctctatagaggaactcaatcaaaaagcactgcagggctgttatacctatatactatctttgcaggctcgagaattgcttatcgcagccaaaactcgtcgggtggaaaaaataaaaaaaatgcaagctcgtcttgataaggttgagcagcttttgcaagagtctccctttcccggcgccagcgtatggctggaggaaattgatgcggtggaaaaggctattataaaaggaagaaatactcagtggaaatttcattaaacgctaccggttttatgatgtccaataggtgttaagcaatcagttcatcaacatttttttcaagaatttgaaaagtttggataatgttctgaatacttttttctaaaagagttatcaaatcttcttgtgaggccttatgaataattgttaataccatttcttgcttatggggaacacactgataccccacaaagctaatatcaggaatcatttcataaatatatgtttttagcagatttccgatggtatgggtttcatcttttatcgtgataatggcctttgttttttcctcatccatggaaaacagcacaagttccggctgcggctcttcaaagttttcataaattttttgaatgctttggattcggccaataatgatccggcaggcgttttttaaatacgtgcgaacggcctggttgatatgtggcagcggcaccgctggaaagcaaagccccaggcggtggtgacgcgggtctgaggtcatagagctttgcttgtaaccgctaagcgccatatattcttttttatccgttgggtactgttcaatgtcaaggtgggaaaaatgtgttttaacggcaagattaaaggcggcatgctttcgtcctatgccctttttaatatagatatcctctataatcaacgattttccgggttgtaggaagccaatctcaaaggtaggattaaaaatcgggtatttaagcttagggcctgccacctggatgagatcgcggctatagatggttttaacctcacagctattgtttaaactccgcagagcaaataccagtgtctcgtttttcgcataaatcggaatgaaattaatgcggtttctaataaattgttccgtcataaacaggtccgtggaatcctcgatcttatacccaccgggcttaatatctagcatataattgggaatttcatcttgcaagacccgcgacaggccgtggaccgcggctctgctaatgcccttaaagtccataacaacattgaccgggacgaggggcaactgctcctcgagctgaaatagttttttggccgcatttttaataaagaggttggaaaagtctatcaaaaacggtttgatttccacgttttggaaaattttttccatttgtattataaatatatctatatatattcaaattatggtagtttatgacttgctcgtttctttaagtaaggaatccatagatgtgctacggtttgtagaggcaaaccttgcggcgtttaaccagcagtatatttttttcaatatccaaagaaaaaactcgatcacgacaccccttctcattacgccgcagcaggaaaaaatttcgcaaattgttgagtttttaatggatgaatataataagaacaatagaaggccctccgggccgccgcgtgagcagcccatgcacccattattgccgtatcaacaatcctcggacgaacagcccatgatgccgtatcaacagcccccggggaatgatgatcagccatatgagcaaatataccataaaaaacacgcgtcgcagcaagtaaatactgaactgaacgattattatcaacatattcttgcattaggcgatgaagacaaaggtatggacagcatgttaaaacttccagaaaaggcaaaaagggatagcgatgatgaggacgacatgttttctataaaaaactaacgacgtaacaattaaacaaaaaataaaaatcattataaaatgaatcttgaatacgtccaagttgttcaaaaatttaatcaagtactcctagaacttaccaaaaaagtatgtaccgttgtgggcgggagcaaacccacctattggtatcaccacattagaagggtttgctcagaatgtccatccatgccgatgagtatgataggtccgtatctgaatgtctataaagcccaaattctaacaagggacaagaatttttttatgaatttcgatcccgcgcataatgagtacacctttatcattcaaaaactaaaagaagcagcccgaaatatgccggaagacgaattagaacagtactgggtaaaacttttatttttacttaaaagctacataaaatgtaagccctttattaattaaagaattgatgcataactaataaatggccggtcgtgttaaaataaaacagaaagagctcatagactctactgtaaaaaacaaaaatgtgatgaatctgttccatgaaattataggctcaaaaggcaatattaattttagcgttgtctggcccaagtttaaaaaaatcaaacagagcgtttatgactacatttccactctttctgtgctggaaaaagcaaacgttatgcaaaactttgaagctgataagaaactgttggaactttttgtacaaaagctgtgggctgcctatgaaggctatttcaaatatcccgagattgaaaaatatgaggtggaaggccaggtaaatttcaatctcgtacctcagtgcgtcctcgaaaagtttagccagttgtataggataagaatcaattcagagcttgtcacactcatcctaaacagctgtgcctttatgagtaaatataacgattatattctcaaaaaagatccctacatactaaccataacccccggcctatgcttttcccccattcccaacttcgaggacctaaattttaaacatctttacaacagtgataaaaattctcagcatgacaaagagtttatcatgtttatattatataagctttatacggctgccctaggagtgtacaatgccatctcgattccagacatcgacgtagaagaccttgaaaatatcatcctatcctcggtgagccagattaaaaaacaaattccgcgctgcaaagacgccttcaacaaaattgaatcttcggtacacctgttgcgcaaaaattttaacacatattacagtgactatgtgggctcaggctacaacccaaccatcattatggaacagtacattaaagacatatcacaggattccaagaacatatcaccacgcatttcctaccagtttagaaccatcatcaagtattaccgcgacatgattgccaccaggcatcaaacgatggacccccaggtattaaacctcgtaaagcacgtcgaaaagaaattagatatgcttgatagagaaaaaaattagtatatatagttatggtgaatctttttcctgtttttaccttaattgtgattattacaattttaattacgactcgagaactatccaccacgatgcttattgtttctcttgtaacagattatattattattaatacacagtatacggaacagcagcatgaaaacaatacatttttcatgccgcaaaaaaattcttttaacgaatcttataataaagacaaaaaatctaatatacatattccctaccagtggctggcgcctgaactgaaggaagctgagagcaagtactggtggggcaattatgatcctcatagcgagcccgttctcgctggcgcatcttgaatatcttcatacgtggcacgtcaccatcaaaaacattgcccaacagcacgggcttgatataaaggtggccattgtggtctcaacatcgcatttaaataattttttgccaatttccggggcgcttaacatcgaatgtataaccttccccagttgcggcatcaaggagatagacctcctatgggcgcgcattaaactatttcaacattactgcgccatcggtgcccgtcttttatggctggtaagtgctgacatcaggccccctgtttcagcgtggccagccatcgccgacagtctaaaaaagggagcagatgcggtcgttattccctacccctcccgatggaacaatcttatacctaccgtcatcaaagaaatagttgtccaccaaaaaaaatgccttgtggcggtggatgcacgccaccttgatacagatacccagattgtaggggccgggatgggctgcatcgtcctaaccctaaaggcccttatggtgcgcctaagtattggcaaacagcccgttaagatactgtggcccgaccttcacggcactgccgagggcattcctctggagggggtggaggttggctggtttttaaacgcttatgcgcataaattaaatatacgctgcctaggggctgatcatattgcgcagcacttaacttaattctttatttaaaaagtccacgcatccagtggcggcctacattaagggcctacgcacataaatatacactggctagaagtacgccttcatttaaaccattgaattatttatataatggctgcaaacattattgcaacaagagccgtgccaaagatggccagcaaaaaagagcatcaatactgtctgctagactcccaggaaaagcgtcatgggcattatcccttttcatttgaattaaagccttatgggcaaacaggcgcaaatatcataggagtacagggctcacttacccatgttatcaaaatgacagtatttccatttatgattccttttcctttacaaaaaactcatatagatgattttattggtggacgcatttatttattttttaaggaactggacatgcaagcagtttctgatgtaaatggaatgcaataccacttcgagttcaaggttgttcctgtaagccccaaccaagtagagcttcttcctgtgaataataaatataaatttacatatgctataccggtagtgcaataccttaccccaatcttttatgatctttcgggaccgctagatttcccattagatactctttcggtccatgtggatatcctctccaatcatatacagcttcctatccaaaaccataacctaacaacgggtgatcgtgtttttatttctggatataaacacctgcaaacgattgaattatgtaaaaataacaagatttttatcaaaaatataccgccgctttcatccgaaaaaataaaactatatatactaaaaaatcgaatcagaattccgctatactttaaatctttaaaaacgtctaagtaataacatttttatagtctactcctagttccgaaataggctgaatttcttttttaagtcctttaaaccaaggatgtgatacaagactcttaaaggaaagccgcttattttcattaattgttaaacattccgtgataaactgttttcccgtctctgaaatgttctcgggaatataattttcccgtttcaggatatcatttaaataaaaattttctgcacgaaatctaaaaagattaaccgcgaccatacctatcgtccacacggttaaaggaagctggtagtaataaccataataataaaattctggacacacgtattcccatgttccaaacatattatattggggacgggtttcgtctaatctaacagcgcttccaaagtcaatgaccttaatgatcttttgatttatgtctataataaggttctcatccttaatatccccatggataaagcccttctcataaatgttttgtataataagaataagctggaatattatttttttggcttcggtttcctcaagttttttaaagtaatgataatgaagtagatcaacactatttggaatatattctatgattagtatatgatacatagcattttcggtatattcgataagcttaataacaccgggagtatcttgcagggctttcaacacgatgacttcatttcctggaatttcttttttagaaacgtacttaaatataatgggttgccctacttgatgacccaaaaagacgttatttctgccaccctcaaacatgggtctcgtcgcaatgaaatacatgtgctgcgttgtggagatcctttccacctttgctgtaggataaaacgcatattgtgcctggggattttttaacatttttttaagctgttgttccggcctggacatgttttattagctttatatataaagggttagaaggtttaatttcaatatatgccttaatgatgggattatattcgtaaaaggtatagcctaatcctacgtctttgtttttttggtaaaaaaactgtttgccctcgtaggatatgctataggcttttacttcggcttttacaagcggttggcagggattgggcaaacgtaaatcgcgttcaaagttttcatgaaaaagcaaagcatttgtgggctgacacatcagacagccgctttcgccattgaaggcacattcaatggccgccctttttagtaaatcgcggaaagcagaattaagatggctcttttcaagccccctttcgtgaaaacgctcatcaatcgttttttgttcctgactgccttcgggaatactataaaacattttttgattagccaccgcgatgtacaaaaaaggctgtacggttttctcctcgggcggtagcgcatcgtggctaccaatgcgtataatgcgcgccttcacttgatcctctcgggccttatcccagtacggctctaggatatgaacctgccgcccgtatttgagatccaatccctcagctcctgttttagagacgagtaaaattttaataacctctccgtgtatattcagcggcgaattccaaagctgctggatcatgtcgcgctctttagataaaattttccctgtaataagcgtaaatcgtgttattttggaggacaggactaacgtatgggtcggcccatcttccgcaaagtttttcaccataagatctttcccatccttatgaaggaggatggtgttgtgcccttcttccaatacttttaggggctgaaggcactggtagccctctatttctaaaaagcgggccacgacgtgaaggcccaattccacaaactgtgagtaaatgagcacagggcccggagacgttttaatattttttagcatgcgtactattttgggactagaattttctgtgaaggcctctttgggcagctgctgaacagcctctgataatttttcatcctcctttactgttagcatttcggacgcgaagatgctgatcatacgggaacgcacatagtaggaggagcctgactcttgctccgatcctggcaggcagagggcggcggcatttattttttcatacattcctgagctggcgtgcttttccgcgttttcaacgtctcgggccagcagatattgcctatactgctcgggtgacatttcaaccttttctataataagaggaagctctgtggggaatagcttgttgagctcattctggtttccagcgtagcttatcatacccactaggcggtttagtagtttgtccgcgtttaaagggctattcgttgttttattgacataagcggtgtagaatctttcatagtgaagaggtaataagattcgcccgcttagcatattaaaacagggcaccatttcaaaggggtccttcgaacacggggtgcctgttaaaaacagaatacgaatatttttagcttgcataatattattgtacagctggcgggcatttgttttatcattggcgctattgataattcctctaaagaggttgtgtgcctcgtcaacgatgagcaggcatccatttagggaccctcccgcctttatgatctgctgccccatgttgtaagcgtctagggacacaaacctgaagcgccgcgagattttttgtagctctttggagtgatccgtcgtttccggatataaaagtttaataagctttaacaaagactgttggaagtttgagtgcaacgacttgggtgcgatcagaatcgggttgtaaatatgtgaaagtgagatggcaagcgacaggctcaaaatggttttccccatgcccatctggtgatagatgaggaggccccgtgtgttttccccctggcctatcccaaatttaggatccgaaaaggcggtgtaaattaaaaactggtagtatttcagggctcgtgcaaagcgggcagtgagtgaggtgtctttgctttcctgaagctctttatatttttcatatacctcttttaggtatgcttctatttggacggggaaggaggtgttgttgtgcacgcaagacatgactcgttataaggatcccatattaaaacttcattagaagaatagggctgctgatagctagcgctgcacttaaaaatggggtagccctttttcttgtaaatccggtgcctgtcgtagacctggctagaaagcgggcttagtgtatctttaatgtccacaacgatgcgtacctttttttcatccgatccctgccgggtaatacgtcccaagatttgctccatgttgtttctgcggggcgttgccatgatgatcgatgtcatatgcttgaaggaaatgcctctacgcccgtagccataggtcagcaagataatggaagcgctgtgtgcctgagaaagagcggtatttgaaaccccgccgcataggagcgccacctccggaacgataatttgaacatctttgaattctttggaaagcgcctgataaaaaatttctaaaagtttgcgaaattccacgaaaatgatgatgccatacggctcatcggtcccccatttgtgaggctcagcggtatgcagggagtaaagccgctttgcctcatttacgacaagttgtatacgcgaaggatcttgaagtagtttatcaatggtggcaatggccgataccttttcattaatatacacagggctaacgaagtcaggatgtccctgatattcgatttccctcacgtacccggaaaaggttgtggtgggacttacagtcctctggggctgtcctagatggtgaataataatcttgtccataccatcgggccggtccaggggtgtagcggacagtcctaatatccgactaagttgtattttccaaaaaattttgtaattctccggcgagtgtaattcatgtgcctcatctaacacgactagaccaaagggctcaaagaactgctcaggcttcttgcgcagggtattaatgattcccacgatgacgtcgtactctttgctcgtcatgtcctttttcttgcacgctgcattattgtaagcagctacacgtaggtggggcaggagcaatgttagctcgtcgatccactgtatttgaatcgccttggtgggcacgatgaccagggtagggtacaaaagtttttgaataatgctgatcgcaatacgcgttttccccaaaccggtatttagatgtaggtaaaagcgcccatagggggacaggagctttttatgaatcttatcgaccatttcttgctggtagttaaatagtggaaattctgtttcaacgcatgggagggcccgcagcgacacggggcgcgtcgtgtaaaccatgttaaacatttcaaactgcttttgcagcaatatgggaaaataaatgtattccccctgcagcgtgaaggcagtttcctgtcttatggctatgtgctttggctgcccgggtaatgcccgcgccgtaacggtgagcgccttaagaacgcgcccgaaatcatgttgtaatttactttgtagcttcttataatttattcctattccagcaaaggatataatggcctccattctcacgctggacgggttatatgcagaggttccaaaattcttaccagaggcgttacgagagggctgtgctggcaagaatcctctaagcttttatattcaacaaattttaaatttaatgggatgtgacggtaacgagtaccatgttctttttaccagcagctccgaggaagcaaatactcatatgatcatggccgccgtgcgtcgccatttgctgcggacgcagcaaaggcctcatgtcattatcggagcagccgagccccctagcgtcaccgaatgtgtgaaggcattggcgcaggaaaaacgctgcgtatacaccatcatccccctaaaaaattttgaaatagatcctgttgcggtatacgatgccatacaaagcaatacctgcttagcgtgcatttcaggcactaatgctgttgtcaaaacgttcaacaaactccaggacatcagcaacgtgttaaaaggtattcccctgcactcagaagtgagtgatcttgtttatcaaggatgtattcaacaaaatccgcccgctgatagtttttcaataaatagtctctacggcttcctgggagtcggtgttttgggaatgaagaaaaaggtcatgcaaggattggggccgctcatttttggaggagggctgagaggcggaagccctaatatacccggaattcatgccatgtataaaacgctaacccagcaaaggccttctatgaaaaaaataaatacaatacatacgctgttcatgaaaactttaaaaaaacatcagcatgtatatctacccatagggggcgtgtctgcagaggacacgtctgcagaaaacatatctacaaaagacatgcctgttgaaggcccgaagggactcccgggctatattttatttagcgttggccgtcgcgccgaggagctacaaaaaaaaattttcactaaatttaatataaaggttggccgtgttgttgacttacaagagatactgtttcgtatcaaaataccccaaaaatactgggagacattattgttcatccaattaagagataatttgaccaaagaggacataaaaagagttatggttgttttgatgcatttagataccatcactcctcgtggctctcttcctcctccgagccactcttcttctttttcttaatcgtttttgtttgttctataataagggaaaagaactccgtgggatcttgttccccgtacaggttatctgcgaccataaggatgcttagaatggtaaacaggtgagaatacataagggtttgcgttttaagaaaaccctgacgttgaatcataattgaaaacaccttgcaaagccgactcatcagttgttctgtaatggcgttaagcattttctggaatttttcttggttttcgggtgtgattttatattcatgtagaaagtgtttcacacctgaggagaagaatctttcctccttcgagagcccatctttgatgatgggaagttccttgatcagggcaaaccattcctcctcttgggcttgcggattctgaagatactgatggcagatatggtttagaatggtgcacacgtagctaataagctctgagctgattctttggttggttttcaaatgttggcgaaagtagtttttcaccgaagtgcatgtaataaacgtcttcattttcttataatatacaacagtatgttgagtctttaatttaaaattacaaggagttttctaggtctttatgcgtataggtgtttctttgtcgtaaattttcaatagccgacattgtttgtgaagcagtgttctgagtagtgactgtcgtgtaaggctcagccggatgagcaggagcactcgcggccgcaggtgcggccgccggcccgccagttgccatgactagtctgtccgtaactgggttgtccgtaactggtttgtttgttgctggtctgtttgttgccggtctgcccgtgactggcttgcctacacttgctgtagtcgctccagctggtttagaggtacctggttgtggagtgacttctacccactgctgatcttgataaggatttataaactgtatatcttcctcctcaatagcagcagcttttttctttcttgaagagaatagatagattagaacgatgataatgatgactaagaccacgatagcaatgagaatagtatacatatgtgtggagaagaagcttggtgtagtgactggtgacaaacactcaccataatgccgcggataaaccggttgaaaaaattcagaatccatttaagatactattataaataatatataaaaatgttgtggcgcaatgaaattacagaatttatggaccaactttccaagtattctcaagaaatcttaaaaacgtttaagcaattgcgtcctagtgaatataaacaatacaatgaatttttaacacaagttacaccgttgctgcaaaaaacccctgaaaaaattccagagttggttgaccatatattcaattacctagacaacgttgaaaaaatttgtgagctcctcgtgaatgctagctcaattattattagttcaaaaatacgagaacaagtaaaacacggaatgagcttcagctataaagccgacctcgactccttggcggacattctctctcaaaaacagtacgtgcttatgcatctttcaaaaaatattgcggccgagtattttaatacgtgtttaaaccaagggaaatccaagttagatctcaaagctgcctctgtattttatagtagtcgttcccgaacggcaagctcagcagaactctatagaaaaatgctatacgcctatggttcaccgcaggaaattaattattatactgaaaaagcccgaaataagacgttggatgtggaggagagcgacagcatggccatcatcgaacgaacggcccgacacaacctttcccttatgcacccgctagaagccatggggcttacctttggggcaaccaacacggacgccgacccggaggatctgaaggacaaaacggtgataaatttaacgctcccgcaggcaacagaaagcatcacctaccatcttaaatccctaatgcagctaaaaaaagtaagtacggcttcaggactaaatacaaacattttgaaagcatttgataatattatttccacccctgtgaaaaaaaataaaatggcctccaagttggcgcccgggatggatgtcgtgttcactagcgataacggaaaaacattttttactaaaaacattttaagcaaaaacatgctagcggggcccaaagagcgggtgtttgcatataataatctcattagtaatttaaataactcctgtttcatacaaaatcacaacgattttttaagacagcaggactcttggcccttctatgacgcgcacaattttaccaacaagtttttaatgcagcctattttttcggggcagacccgtcctcggcttcagggagccatggaggcggcgcatgtggaaacgcatctcacggcatttttacaaagtattcagccctctaggccacaagatccctctgttttggcttcccccaagttatctgctctaatcttgaactaaaaacagcctttcttggacttaaatgatggtctaccagtttttgaaataacttagagaactatgaagattttcatgaaatttaaattagagatttgcaaaggttacttgcggtcattttctgttgaattaaataattattcgaatagtataatgtctgaagatattcgtcgtggtcctggcagaccgccaaagaaaagggttgttcccaactttgagcgcaagggcattctggaaaaaccagttcggccacaaagccgtctcgagttttcctatgataacccgctgatatttaaaaatctttttatttactttaaaaaccttaaaagtaaaaatattttggtgcgatgtacccccaccgagattacctttttttcacgtgaccagtcgcaggcaagctttgttattgccaccatcgacggaaaaaacgtgaaccattattacgccagtgatgtcttttggctaggcatcaacagagagctcgttgaaaaaatgtttaacagcattgatcgctcttttttaaaaattaccatcgttcaccgctatgacaagcctgaaaccctgttttttatctttacggattttgacattgacaaggagtgcacgtatcagattacggtctcggagcccgagctcgatatggaccttatcgaaatggaaaaaagcatcagtgaagaaagactcaagaactatcctctgcgctgggagtttacctccaagcagctcaagaaaacatttagcgacttatcaaactacaccgagctcgtgaccattgaaaaactcggcggcgatacgccgctgcacctgtatttccaaaagtttaactccatctcataccacgagatgtataaatcttccaacaagatcaacctgacctcgaccattcctaagtcgcaggtgttccagataaatgttaaaattgctcacatcaagtcgctggcctcggctatggtcaccgacaagatccgcattctgtgcgaagaaaatgggaacctaatctttcaatcggaaatggatgcccttatgttaaatacgattaccttgaacaccacgatatagttcggtaacattagatgttctaatatttagcatctaaataatacgctgtagtccggtcagggttgcgtcacagttttcccatttttttgcctcgtcggcggtggccaccgttgccctatcatttacgcccggtaagacaaagctaaaggcgttcagcggggcttggcaatgcccgcccagcgtgaaggagctcggaggattttgcgcatcccgaaatcccttagccatgttgtttaacacttcggttacgtcaatcgagtgaagggatcccttgggatccgtgaatgtaaagacgcagtttctaaagcgcatgtatgcgatggacgattcatcgggggttttgaaggtaacagtgttccccttgctgtacttaaagggggaccatccggtaaaattataccaaatgaaagcaataataattaaaataaccaacacaatagttatagacaacacaaagtctgtagtgccgcccattattaaataaaaatattttagaccgccggcttaaaatttacttattgctcatagcttaagtctattttattcatagcttaagtttattgctcatggcttaagtctattgcttatagcttaagtctattttattcatagcttaagtctattgttcatggcttaagtttgttgctcatagcttaactccattactgatagcttactgatcatgacttaaataaaaatattttgcccgcttaaaaattgtttaggtttgaaaaaataagagatggagggggcaacttatcgtcattgtgtttacccccactggaagacatcaaacggtaaataattataagaatcaaaatgattaatataagggttaaaaaaggatgattcatcacattaattaaaaacgtatttataacgctgttgcagttgaaattttggtataggtcggaaatattgcccgagcctccgtattctgcaatgttctgacatatggtgagtccggaggggcactgcttgttggtcaaaatatttctttgctccgttgttttataggcatttttatttccattacacggagcaaacgcacattcagcccatagggtgccggagttcacacaggcacaatactggctatacgcatactcatcctttgagcacaatccctgtttatcgcatatgctcccaataatattgtcatcctccgccgtttgttgatttgtatgcgagcgtaaaatagcggcccaggccttgggctcctttttttgcagctcggaaatcgaagggcctgtacagctaaagtcgacccaaatatcattgcatttcgtggaaactggcatgcaagacataattgaaataattaataagtatatatcatggcaacaaatttttttattcaacctatcaccgaagaagctgaagcatactacccaccttccgtgataacgaataaacggaaggacctgggggtagacgtatactgttgctccgacctagtgcttcaacctggactaaatattgttcgcctgcatattaaagtagcatgcgaacacatgggcaaaaaatgcggttttaaaatcatggcgagaagcagtatgtgcacccatgaacggctgctcatccttgcaaacggaattggtttaatagacccgggttatgtgggcgagctcatgctcaagatcattaatcttggcgacaccccggtccaaatatgggccaaagaatgtttggtgcagttggtggcccaaggtgaccatgtgcctgaccatatcaacatcctaaaaagaaaccaaatatttccgctgtttgcgcctaccccaagaggcgagggtagatttgggagcacgggcgaggccgggattatgagaacttaattttattttttttcttaacataatgggaggctctacaagcaaaaattcctttaaaaatacgaccaacattatcagcaattccattttcaatcagatgcaaagttgtatttccatgttggatggcaaaaattacataggcgtattcggtgatggaaatattttaaaccacgttttccaggatttaaacttatcattaaacacaagttgcgtgcaaaagcacgtaaacgaggaaaatttcattacaaatctttcgaaccaaattactcaaaatttaaaagaccaagaagttgcgttaacccaatggatggacgcaggaactcacgatcagaaaacggatatagaagaaaatataaaggtaaacttaacaaccacacttattcaaaactgcgtttcatccctgtcgggtatgaacgtgctggtggtgaaggggaatggcaacattgttgaaaacgcaactcagaagcagtcgcagcaaatcatctctaactgcttgcaggggagcaagcaggccatagacaccacaaccggcatcactaacacggtaaatcagtactcacactacacctcaaaaaacttttttgacttcattgcagacgcaatttcggctgtttttaaaaacatcatggtcgcggctgtagttatcgttctaatcatcgtagggtttatagccgtcttttactttttgcattcacggcaccgccatgaggaggaagaagaagctgaaccactcataagcaacaaggtattaaaaaatgctgccgtttcgtaataatttaattaaaagtaaaaaaaaaaggtattgttatagtgatggcagattttaattctccaatccagtatttgaaagaagattcgagggaccggacctctataggttctctagaatacgatgaaaatgccgacacgatgataccgagcttcgcagcaggcttggaagagtttgaacccattcccgactatgaccctaccacatcaacttccctgtattcacaattgacccacaacatggaaaaaatcgcagaggaagaggatagtaattttctacacgatactagggagtttacttcactggtccccgatgaggcagacaataaaccggaagatgacgaagaaagcggtgcaaaacctaaaaagaaaaaacatttgtttccaaaattaagctcgcataaatcgaagtaaaaattgaagcgaaaaaaagtagaaaaaaaatgtttggagcttttgtaagccaccgtttgtggtcagatagtggttgtacgaccacctgcatcacaaacagcattgctaattatgtagccttcggcgaacaaattggatttccctttaaatcagctcaggtatttattgccggccctagaaaggctgtgataaatattcaggaagatgataaagttgagcttttaaagatgattgttaagcacaatctttgggttgttgctcatggaacctacttagatgtgccctggtcccgtaagagtgcgtttgttacacattttatacaacaagaactacttatatgcaaggaagtcggtattaaagggttagttttacacctaggcgctgtggagcctgaacttattatggaaggactaaaaaaaattaagccggttgagggggttgtcatttacctggaaaccccgcataacaaacatcatacatataaatacagtacaattgagcagatcaaagaattgtttttacggatacgaaataccaggttgaaacagattggtttatgcattgatacggctcacatctggtcttccggtgtcaacatctccagctataatgacgcggggcaatggctgcgctcgctggaaaacattcattccgtgatcccaccaagccacattatgttccacctaaatgatgccgccacagaatgcggaagcggtatagaccgacatgcaagtctttttgaaggaatgatttggaaatcatatagccataaaataaagcaaagcggtttatattgttttgttgaatacgttacgcgacaccagtgtccggctatattggagagaaacctcgggtcttccatgcaattacaaaccgctttaaccgcagaatttactacattaaaatcgttattaaaataaggatgagttttagcgaatgtcccttagttattagtgcatgcaaaaaatttctacaaaagcgtattacaatagagaatgaagcacttataaatgccttaataaccgctttagcgcagaccagcacgttgaatgatctttgtttattacctattcaaacctatttgcttagttataaaaatgcttttgagtggatacacttcgtatgtattgcaatcaccactattttggataataagtataactggaaggactgtacggtagatattaattatatttttctccatgtaacctatatttacaatattaaaaccaaggaatacctagactactgttcttaaactttattttttctatatttacgccaaagagaatatttaaagttttttttgaaaaaaaataatatatgtagataaaattcagttacatgatatatgtgtaaacatgtgtggtaaacaacatatggttatgctttataagataaatgcgcataatatatgtaaacaaaatatggttatgtgttaaatgcatataaatgtattttaacgtatatcttgtgataatggatatatgcatttattaaaagaggctgtatttattataaatcttgctaaggatgccattgtcaacatatatcccatgttggacaaattgcgttgcgatccagttcttttttttttgattttgtttaatgctatcctttttgaagggatggttgtccaccatatttattcgatgttcaatgaataggtctgctttttcgtaaggcagtgaaggtcgttccaagactccttgaacgaaggacgtgttttcttggatccacttaaaaagcacgtggcattcaaaaacaggacagtgattggatccttggatatgctttggacagccaatgcttgaagagatgtagtcccttttctttaggacaagcttctccacgctggggcaacagagatcgttcaagttctggacggtcgcatttggaatgttgaaacttcgtatccattcaccctcgggtcctcccttatgaagaaggagtatttgctcatggtccttagtaatcttaaccaaatgttggaagatcatttttttacctgctttaaaggcctgaagggtgtcagttggcaaagctattgaattcgggagtgggctttcatcaagcgtgaaatggtgaatgtgacgcgactggaaagaaaacgaccgttgatttattttttcaaagattgggtcgattccgccatgaaagaacagctgcaagattttagaaggcgtatttttttcccaataaaaaatgaccacttctcgtgggattaaaatcgtctgtgtcccattttcattatataattggcccataaagccatcaacgtcaatcaacaccaaaagcatggtatagagagcttttagaaccggagttcgttaaaaaaatacaaagttcgtttaaaacgtgtaatgttactaaaaaaatgtaatgtttaaatgataatgataccacatgcattaatgaaaaaaacttttaaatttttgttttaatatttgcatgaaaatggaaacatttttagtctgtttatttcacaatgcagatggtttacatcaacagattcaggaaattttgtatttattgcggatgcatatttacgaaacaaatctttacttaaagcaggaactatcacggcttatatatccaaataggcaactttcttttgtgttacttatgcccctttcccttctaagaaactgggatgacattgaatatttaacggacgttgtagatgataagcagactctacattacgcggcaaatttgctgacaaactacgttctacatctatccatgtttcaaaagctgacaaaaccatacttccttttagcggtcaagcgggtcagcgaaaaactcaacaaaaagcagcgacattcattttacgaggtattggtaacctccgaaaccttgaataattatgaaaacctatctaaaaacattttaaatacgttgatgtttgccgtgcgctacgtatttaaacctacgccgaactattcagaaattctcgcagagttggaaaaaaaaaataaaattcaccatattatttttaatatggtaattacggattttgcgcaaatccgtgaacaacaaatggataaacatctgtgtgaaacaaataatgagcttcgtcaggaatgtaaagaaactatttttgatttaaaggtggtaggaaatgtttagccaataaactcatgcccgcattttttacaggtacaaaatatcgtggatggctcatcgagggcgcgtgtttgtacttctctgtaggtacacatacgctgcttgcagttgggacacttataaagttgtgacgtcttttcggcgaccttttgctgcgaacgtagagtaatttctgtcttctcctttaaggcggcagaggggcaaagctcggcgaacgtcatgctaccaattgcctccggttttagctcgccagaaattagcttattaagggcatcgttatcctgttgttggtgactttttttttcgcagttaataatatgattgatcgtcccacaacgggttgaatattcttctaaaaaggttttttcttgttgctggtacgtataatgataacacgaggcctcgattttttgcgcgtattcggtgcataaatcagtatgttccttaaaaaacatatgtttttgaagcgttctaaaaaacatcatttggatgatatcacgcatttccaaaataatatagggttctagtcttttggaatctttcataactagatcggtggtaatattcttagtcatacaatttattaaaaatggtttaatatattgtaaatattttttaggcgtgtcagcctgtaaaaaacattcttgttcaatcttatttgtaaggatagtattttgcaaatacttatttagcaaaaatacgatagaatcgcgggctatatgcattttcatataattttttttttaaaatttaatacaaaaaaaagaagtatagactcttcttctagtccggttagttcgttggttgcctcaacatggagactcagaagttgatttccatggttaaggaagccttagaaaaatatcaataccctcttactgctaaaaatattaaagtagtgatacaaaaagagcacaatgtcgtcttacctacaggatctataaatagcatactgtacagtaactcagaactttttgagaagattgataagacaaataccatttatcccccgctttggatacggaaaaactaattgtaaccagtagtacatttaaggatagtttaagcagtaaatgtagaataacacagttaagcaataaataacaagtatataggaatatataggaatatatagaaatatatagaaatagctaagcttaatactaattcagctttttttttaactaaaacctgaatagatgcgaagtagcggacatatacatactaaaataagccatacatttactttcttcttgaacatgaaacctttttttcttctgttgttggtatataaacaataggactgtttgctgaggttgtatgatcttctacaactgctgtctcaggatgacgatgtttttttaaactaaaagtgtaggatggaatgagtggaatatagttatggctcgacttatcctgtttcgtacaggaatattttttacaaatagaacgcaacaagcatatgaataaaaacagaaatgatatacaggagcataaaatagatatgaacactaaggggtagcagcttttataacgttccgtatttttcttagctatcaattgatttaccgtaatatttatctcgggaaactttgttctacaatattttgtttggtattccagaaactcatgtcctggcttattcccgcagcttaaaaaatgatacaaaaatgtgttattgttactaaaattaattcttcttaagaaaaactgcggaagacgctttaggtacgtctgttcctgttttagtaggaagtagtataagggacaatttctttttccacacattagattattgtaatataggtaggttggggtgttggagcgaataagttttctgagtatgttataatctatgacttgtaaatcgttataccttaggtccaaaaacttgagttctttaccaaagccacctgcaatttcagaaatatttttcatcccgcagcggataatacggatgtcctgaaacgtctttaaaatacttgtattgtagtgaatacttatgttatttttttgtaaataatctatgtcatgacaagtgcatgaaatgccagcagcattgcttggtatagtattatatgcaggaagaactatactactattgagaatagtcacattgtacttataccatgtattattttctgatataaagtatttgcaggtgacctgtggtttaatcctacctgttaagccacttcctaaaaaaacaaaaaatatgaaaacccttagcatcctgtatatactattaaaaatttataaaattttctgtttaaatttcatttagacaaaaaaaataatatatatacatcagcaagaaattatatacagattatataattttctgatttttttttgccacaataagcatcattatatgcattaaaatctcaatactaaacactaaaatctaaattctaagcattaaattctaagcattaaattctatgcactaaactgtaagcactaaaatctaagtaactaaaatcaacactaaatgtatgcaacctaaaatgtaaagcattactcatcatcctcctcttcttcatcctcatcatcataggttaagatatatgtgtcatcctccatttcttcacattcatcttcataagcatcactgggtattggtggaacattggatgcagcatttttaaaatattctatgtcttctggtgaacactcatctaatgattttttgacagtccttttaacttccatgggatatgattccaaatcctctttatataagagtttacggtagcttttagctgcatccacatttgctggagaatctggatttggctcattgagcagtgaaattacactaagaagaatggtatcaatcttttgagccggagaccaagtcattccctgttcttcagcattgtctccgtgtaagatagagatacatagttttccatcagagtaaatattaggatgccacatttcagaggtgaatgttaatctgggtggtgcatatgggtattctggaggaaaggcgatttttgccttgaataagcctccctcataaaaagtgtcaggtgggccccttaagatcacatcccattcagtcatatccttctcattcaccgaaattttgaaattctcagagggattctctatcaggtgtctgtactctgctattaaaaacctggaaaccatggttatttaatattaattaaattccctggtttattcctccttaaaagtagatgaacctcttttgttttttattgggttcatttttactaaatttatgaactggaaaaaactttaacggcataattatcaaatgcgaagggggatccgtataaaatcctagcttgccggtaatggctattaagttaaatttggtaccagtaacactaatatttaaaaagccctgatcattaactttccacattaaaagattattatattcgaatgtttgtccaatatggacaactttgtcaccagatgttacatttgatttggttgttagtggctgaagcttggcacaatcaaaaataagcccattaacactaagatatagaggagtgggttgatctattttctcatagtttaatattccatctttccacgtaatagcttgataattatccgcagcaatgagttgaaattttataaatagtacaggggttttagttgtcgttatacatttaaagggtgttttataaaaataaaaaataataattgttaaaagtatgataataatcgccaaaataatttcatacattttttataagaattatacatagtatggtatttaaaatattagctaaatttaaaaaaacttcatgatttttaaaacagggaaaaaggggattaggttgaataaaaaaggtaagcacttgtctatatattttttttacaatgttgccttgagtcgcatttttaactggctggggagtatcagagtggaatatcactgtagtaggtctataaggtcttgttaaaatatgatcggtcattgttttcgtactagtgtcatttagggtcgacctgatagctcgatataaagttataggggataacctatcaaatacagtcttatctgtgctgaaatgtatatcgtcttctttatcactaataatattaggaatggctgtcattaaataattactacttgttgttgtgggtgaaatagttgtactggtattattggaaatggctgtcattaaataattactacttgttgttgtgggtgaaatagttgtactagtattattagaaatggctgtcgttaaataattactacctattacaagtaaactaatgctaactacatttttaacctcaataaacctaaaaagccatactaaatacctaaacaacatcctgttataatatgagcagaaaaaaaaaataagtataattagggaattattcttattcgcttactattaagaataattcagaatcttatttagttagaaactatcataaagtgaataggactcatcgtcggatgaagattccgtttcagagatagtttctttttcttcctcagaataatctgttcctacaatagaatcggtgtcatcctcagaaagagaagtatttaaatatggactatctatagcaatatcctcttctatctcgcaatcctcctcctccatttccatagtgtgtaggagaatatttttatcatcatgctcacttctttttttgttgaaagatgaaccgtcctcaatacggttcatgttaagttccttcatcttatgtataatttccgtaatccgtgatgtttttgacatgtaagatggttttaaggttatatccacaataacaggagaatctctatcattttcatttgataaactttgatctttgatttcttcgtctaaaattcttgtctttttttgggtactagatgaaatagaggaattcatattctgaaacgatatatcaaggggagctggacgcttttttccaattaaaccgtttttcgagatactatgattagatgaatgatctttagccaagctgtccttggatatactatagttagatattttacctttaaataatattcttctatacaagttattcttaggtaaagaattagtatggattcctatatttttatctgaaggagtgtccatatcggagaacgtcctcttacgaatattttgaccacgagccatttcatccactataggcagtattttggctggctatggttctttgttgtgacaattctatgagatttgattgcaaatcaatttttagttttaaatatattggtacctaggacaaagaaagtatatatagccaataattattccactaaattgatttccagactgatgggtatggagccatgttgtctctgcagacgatcgcaaaaatggccgtagcaacaaacacctactccaagtatcactatccaatactgaaggtctttgggctgtggtggaaaaacaatacgctaaatggccctattaaaatatgtaaccattgcaacaacataatggtaggagaatatcctatgtgttacaatcatggaatgagtctggatatagctttgattcgggcagtaaaggagcgtaatatatccttagtccagcttttcaccgaatgggggggaaatattgactatggggcactttgtgctaacactccatctatgcaaagattatgtaaaagtttgggagccaaaccaccaaagggccgaatgtatatggatgctcttatacatctttcagataccttgaatgataatgatctgattagggggtatgagatttttgatgataatagcgtgttggattgtgtcaatctcatacgactcaaaataatgcttaccttgaaggcccgtatacctctcatggaacaactagaccaaattgccttaaaacaacttctgcagcgatactggtatgccatggctgtacaacacaacttaacaatcgctatccactattttgataatcatattcctaatataaagccatttagtctgcgctgtgctttgtattttaatgatccctttaaaatccatgatgcttgcagaactgtaaatatggatcctaatgagatgatgaacattgcttgtcaacaggatttaaactttcaaagcatttactattgttatcttttaggggctgatattaatcaggctatgctaatgtctttaaagtatggtcatctttctaatatgtggttttgcatagatttgggggcggatgcctttaaagaggcaggggcgcttgctgagaaaaaaaataaaagagtgttacaacacatattaggtcttaatatctttaagcgagagttgattcccccctgtaaagatcctgatccttatcaaatccaaattctgttaaaaaactacattctaaaaaatgtctcaactgtttttacatattattgccagtagccattgtttatatcagaaaataacccatttgtttatctttttttgtggggcaaccattaagacccgacgcaaaaaaagattaatcttttatcagatacctaaaacgttctataagggagtctatgagatggatcatattttgatggtcatagtaagaagcaagctttttggcgaaaacaacggagttaaagaatttaacccgctcatgtttggataggacttttaacagcgagccaaaacagtatttaaaaatttggcaatagtttttttgggatgcaataaacaaacacttgatcagtgcccgcttcactttctgatcagacatgtttgccgcataacaggcctttttaaacttagtaatataattatgttccgcaagcaccattaacaagggaacgatgggaagctgcttttcttggtgaaatttacgtaaatattcgatggtcaccgcttggacgactgtgtaatttactaagttagaaatgatagctttcatggttgtaaaaatatacataggattttctttttctgtatacagtttgaaaagcttatgattacgtgaaatgatggccatttttaatacaagatggtatagtgtatctttaggtaaaaatgccttgcaagccgcgatgatgtcgatgttgtctccatgaacagcgatagaaactaatgtttccaatctaaatgtttttatctgcattaatagaagaatgcagtcaatgttattatacttaataatactgtaatacaccgaatcaatgaccgtcatctgagaatcaagctgacttattagtaaatttaacgtttttttggaggcatgacctttgatcgcggcactaagtgcacacagtatagcaaaattgttaaatacattttgatttaggagaaggagtaatattttccttcggttatagtacgcagcatctgtgatgattattggccgataaatgttaaaatgtgttaacagctttttaaaaaaacggaagtaatttttttggatcgctgtttgcatcatcgaaataatgagataatcagggtatataatgggtaggtcacatgctacctctaacaaagaatagtcgcccaatctaaaggctgtgttgaaaagcgtactatcatcatacgtatcgagtacccctgctgttacaaaccaagcgataagatgaatgtgccgttccttgcaagctatcgcaaatagggagtttcctatggaatgtcgaataatgtactccctatttttttccaaaatgtttggaaaattgtatagcgttgcggcatacagtagacactccattctggcgttataatttttacttttacatatgaataggtggaagaactcgaataattcttgagaacttgttaaatgcataatatggtgatattttggtgtcgttaaatggtatgagaaaatgcattctaatacatcttttcggttatgctttagcgcctgagctaaggcatattcaggctcgacccataggactagtgtttctataattgagatattcgcctgctttgccagggcatactttaagacgctccggttagaaaaaatgttgttatgaagatggataaccgtatccatttttacgatgggaccattccagtatagtcctaaatgctgtagcagatcttttgttagttgtgaagcgttctcgggtgttatataaatatgttgcagggcttttttctgtaaggagaacatttcgtcgtaatcgtacaaaaaaaaattaaaatttgggcatggatgattcaaacataacaaaatcaagattttataacagtttgcattaacctatacatatatgcaagtaaatgagatattatctatcataacgaatcaagggatatttgtatatatcaggagtttctgaaataaagatatgaagattatcatagtagtatccatcaatcacaatgcaacttcctttaaggcataatttagtaaacttagcactcccattttctggatgctttacaactaacattaaaaactcctcagtcatattatctgtaataaaataagatcctcctggagccatttgtagcatgtctcttattcttacaaaatcttttttgggatggtaaaaactcagcagtttcaaactcttttttagttttttttcctggtatttaagccatttgttataaaacagttttcttatgaaaatgcatttgaaaatattgggaatgtttaaccatgcttcttccgagcacatctccagatacttactttctttgtttcccatgtctaatttattgctcactaagttagtaatgaatctattttaataatctactttactaatctatcttaataacctatcttataatctatcttaataacctaattataacctatttataattggctaatgttgccggcatttcatgcctatctaaacaactcctactaagcaatctactattacatatatagattcactttttatatttgtaaatcatgagaattataaaatcattactcatttttattgtaaattagtgggtatttgtaaaaatcttcaaacgttttaagatagttttctagagagaagtaatctttgccatcaatatataatgcttttcctttaaactccagttttgctatgtttagtgagccgtttctagatctttttgggcaataaatagattttcattggttgtatcgtccgtaagcagaaaggtaccactaggcacgttaaaaaacatacgttctatttcatggtcggatttttgagaatagaaaaaatctaattttttaatccgcgttaactcttttttatcaatctttccagactgttttatatatactttattgcaaatcttacaatcctctatggcttcattatacttattttgcttatcctctattgacatgtccgtatttgataggtaacttccgttaaggcggttccccatggttttagatagatttttaattcagttgtatacttttattatgaggctaaaatatagaagtttgatcctaaaaaaataaaaagattttgtacatttatttatggtttatagcggtatagaggccgataaaaggtatccgggtagtctcctatgatatcgtcaattttggtataataacagttgttatggtagtattgtccaaaccgagtatgtatgcgccggtgaagcgtctgcccgctaatggtacagttccaggttaagacaatcatatcacacccaaaaagagaggaaacagcataggtgcccaaaggtttattatataacatacgccgcatatattttagttttttttctccatggtaataatcacaggttttcatgtcctgcttaataggatgattccccatgtatgataatatataataaatttagtttttagctttttcaaaaaattgggcgctcgaaactaaatttttcttatcacagcgtttggagaaagcgtatttaaagatatatcttcttctaacaagactgcaaaaaaaatcttaccccttatttttataatgttcatcatagcgtttgaagatatcagaaggtgccaggttttataaaaatatcctttaggatttataacgatacaagggtctataaaatatatgcgggtataatcttataaaatcatcgattttttcataatattctccgtttatacaataaagatcataacagatattgatgcgtagatgcattattcgcgtgttcgttgggcagctaaaggatatcacaacgtagttttttttaagaaaagacgaaactacataagtccctaagggttcattgaatagtaaacgccatatttgttttaaattttgttgttcaccatagtagtattcgcactttttcaagtcttttttaataagcctattccccatgtatgcttataaataaaaatttagaaatgtgctatattatttgttgatgaatcatgaacacgtcttatatgttgatatgttactttaaaaacatttgtattttcaacagacgcgttctattcttattaagaatgatgccgtctttattttaaaccttggtttaaaatttaaagaagtatttataaactataatcatgggaactttttcagtaactgcctctgcaaaaagtgacgatgctgtttgtaagtatttagaagaaccaatagatgaaaattacagaaacatattaagaaatgagcatgttaaaaaaaatttaaatgaggctctgaatcgacatattactacctataatccagtagttgattggtgtaataactattcaacattttcatctcaggatttcgatgaatataaaatttatatacatagcgatcttatggatggacgacctcgtccaaaaaaaacatggtgtgtcatcatgtaatgtttgttagttttatataaacgcaaaaatattcttctaggagatgttgatatactacctattgaattcaatatattaaagtacatttctggctattcccattacggtattattattactatttttaagagctagatgtggatttaagtaataataacattctcccgttcctcctagagacacctcatcaaattcccatcctatgcaacctttatgttgtaaacataatgattgacagcattcatcttcttttgaccaagtcgtccaaatcctaccaagatctatacgtgtttttccaaatggagattgaagatcagcagtagtggcattaaacctataaaaaccaggtgcataatcacatgaacggatcgtaggatctaatttaatatcttttatatcttgttttactgcttctagacaacttttatcagtacatgttccacgtacacagtggtgtcctttatccttacaatccgtatctgtcttacatttttttttcggcggtttatgtttcagatggtaaaaacccagtattaaaataatcacaagaataattcctataagtacttgaacaacaggataaaacattttaatattaaatatattttttaattaaatgaatagatttaatccaagtagtattaaaattttttagaaatagtgttctacaaataatgaaatgaatggtccaaaaaaaataaggtgtacaataatgtaatatattgttaggctaagtaaatttaatattttaaagtatttggaaaaatattttttaacatatgatgtctaggaatattttttagacatttaaaaccatatagttactttatttattacactgaacttgaaaagacttattacctaaaatattaatagatgaagtaatattgtgtaattgagtccataacatgggtgggaaacaaaaatctcgtaatatgaaaaataaacatcctaaaaagagtgcaattgttataagtttatgtaactttattttaaagtaagaatataaaaatatgagtacaagaggaataggggccattactaacattggctccaacatcctgttgtctacaaaaaaaaatattttttttagcaaaaaaaaatccatggaaggatattaatacacataattatttgacatcacattagtgtacttaccaaatagtaatatacaaccatcctaatattcacctttatgaaatgatcccaacctatacggtaaaatagtataggttttaataaagaaaaaagatattctgtggtttttatttttgtatagtgtgtgaatacaaaataaaatcccaaattttaaccttttctttttttttctatacaggatgttagaaatagtattggcaacgctgctaggcgacctgcagcggctccgggttcttacccctcagcagcgggcagttgccttctttcgagccaatactaaggagctagaggacttcttatgctcagatgggcagtctgaggaggtactgtctggcccccttcttaaccgtctactagaaccctcaggccctcttgatattttaaccggatatcacctatttcgtcagaatcccaaggcaggtcagttgcgcggccttgaggtcaagatgcttgaacggttatacgatgctaatatttacaatatactgtctcggctgcggcctgaaaaagttcgcaacaaggctattgagctatactgggttttccgagctatccatatttgtcatgctcctttagttttagatattgtacgatatgaggaaccggactttgctgaactggcctttatttgtgctgcttactttggtgaacctcaggtaatgtatttgctctacaaatatatgcctctgacccgcgcagttcttacggatgccatccggataagtcttgagagcaacaaccaggtagggatttgctatgcttacttgatgggaggcagcctcaagggactagtctccgccccactgcgtaaacgtctgcgcgccaaactacgctcgcagcgcaaaaagaaggacgttctttcaccccacgacttcttactgctgctccagtagctttttttgccgcaggagcaccgcggataggagctcctccacgctcgcgatccggcgctggaagcggaaccgatcgaccgccacctgctcccagggacccttgcgctcgatgtcgtcggcttcccacacctcgacggctgtggcaaaatggacatgcttcgcgtcgttcgtccgttttttgcgccgcctccccattattcttcctgtaagattagtgtttaatacctataataacataattttaagatttaatataccaaaacttaaactatttttgtatagtaactattagcatgtctacacatgattgttctctaaaagagaaaccggttgatatgaacgatatatctgagaaatcagttgtcgtggataatgcacccgagaaaccagctggagcgaatcatatacctgagaagtcggcccgcgaaatgacatcatcagaatggattgctgaatattggaaaggtataaaacgtggaaatgacgtgccatgttgttgtccaagaaaaatgaccagtgcagacaaaaagttttcagtatttggtaagggatccctaatgcgctccatccagaagaataattaaaaaaaatattttttttagcaagtttttaaactatttaaataaatgtggtaaaaaaattcacataataattaaagtgaacgtgttagaattaatatttttttataatcggatataatatccattaaatcaataaatgatagtgttgctaccacactaaacaataacaaacagaaacgcacgatacctttcctcatgatttataatagcgtgttatctaaagatttttttgaaaaaaatattaaattttagttgattatttttttcagttacaacattgctttagaaaaaatacctaattactacatagcaaataaagcgagcgcattgttacaaacaacatttttttgcgcctggatactcctatatatgagaactataatacggtatattaatcctattaccaacattgtcaataatagtatgtaggcaatgacatactttaaataccaaatatccatggttatttctaaaaatcttgaaaaaacgttaaattttagatcggtcacctacgacagtaatactaattttaataattgatgactgaaatcataatataatgccgtgcgaaaaataattatttttcggttaaagataccattacataaaaaatatgccatctactctacaagtgcttgctaaaaaggtattggccttaggggagcataaagaaaatgaacatatatctagagaatattattatcatatattaaagtgttgcggtttatggtggcatgaagctccgattatactttgttatgatgggagtgagcaaatgatgataaagactccaatctttgaagaaggcatattacttaatactgcattaatgaaagctgtacaggagaataattatgaattaataaagttgtttactgaatggggagcaaacatcaattatggattaatttccattaataccgagcatgcccgggatctatgtcgaaaattaggagctaaagaaatgcttgaaggaaatgaatttatacaaattatattcaaaacattagatgataccaccagtagtaatataattttatgtcatgaattattcaccaacaatcctcttttagagaatgtaaatatgggggaaatgaggatgataatttattggaggatgaaaaatttaacgaacctattattaaataatgactctattagtgaaatattaactaaattctggtatggtatagcagtaaaatataatcttaaggatgcgatccaatatttttaccagagattcatggacttcaacgagtggcgagtaacatgtgctctttcttttaataatgtgaatgatcttcataagatgtatataacagagaaggttcatatgaataatgacgaaatgatgaatctagcctgcagcattcaagacagaaatttatcaaccatttactattgttttctattggggggctaacatcaatcaagcaatgttaacctcagtattaaattataatatttttaacttattcttttgtatagacttaggggctgatgcctttgaagagggtaagaccctggcgaaacaaaaggggtataatgaaatagtggaaatcttatcattagatatcatttatagtccaaatactgacttctcatcaaaaatagaacctgaacatattagttctttgttaaaaaacttttatccaaaaaatctgttcgcttttgatcgttgcaaccccggtttatattattcttagaggaccgctacaaaaattatttttttttcttgatcaaagctccaaaataattattagattaaagtcgcctatagcagcagcccactccaaaaaaagtattttatagtacaaaaaacacgaaaaatagtttgcggccggcggcaaactatttgttgttgtctaaaacttaatgtttttttaatatttttaaatgcaaccatggattgttggactatcagggagaagaactatagctacatcatattgtcaatactggtaatactattaatatggtatcttatacttaactattgtcgatcgaaaaaaaatgcagttacaaacaacatgccgccaccatacacggtgtcaagtagctgttctcaataatagggttgattgacgctcttcgtaataatatgttgattgacgcatcataaaatgctgtggttgattaatatgttgattgtcgcctactttattatataagtaatgatttttgtataaaatacgggtttgtgagggctttattttttcttattagaacaaagcatgcaatttaaggcctacagcaagagtaatttaacacctacaacagtaattttaaggtcagtaataatgtttaattaaggcctgaccactaaaacttaaacgattttgtaaaaaaaaatgtctactccactttctctacagactcttgttaaaaaagtgctggccacacagcacatatctaaagaacactactttattttgaaatattgtggtttatggtggcatgaagcgccgattacgatttgcattgatgaggatagccaaatattgataaaatcggcaagcttcaaagaaggcttatctttagatatcgcattaatgaaagtcgtgcaagaaaataaccatgatttaatagagttgtttaccaagtggggtgcagatatcaactctagcttagttactgttaatacggagtatacccggaacctttgtcagaaattaggcgcaaaggaagctttgaatgaaagggatattttacaaatattttataaaacacgtcatcttaaaactagcagtaatattattttatataatgaattgttttctaataatctccttttccaaaatatagagagattgagtttaatagtttataggggcttgaaaaacttatcaatcaactttatattggatgatatttcatttagcgaaatgttaactagatactggtatagtatggcgatattatataaccttactgaagccatccaatatttttatcaacgatataggcattttaaagattggcggcttatatgtgggctttcttttaacaatttgtctgaccttcatgaagtatataacttagagaagacggatatagacattgatgaaatgatgaagttgacctgtagtacgtatgatggtaattattcgactatttattattgttttatgttgggggctgacatcaatcgggcaatgttaacctcggtaataaactttcatattggtaacttgttcctttgtatagatttaggagctgatgctttcgaagacagcatggaactagcaaaacaaaagaataataatatattagtagaaatattatcatttaaaaattattatagttcaaatacctctcttttatcaataaaaacgacagatccggaaaaaattaatgccttattagatgaagaaaagtatgagtcaaaaaatatgttaatgtatgaagaattatctcattgatacaaaattattttttataacagaactctctgatggtgacaaatctccgataggaatatatgacgtaacataattatttttttcgcccagaaaaaaattataaatgttattattgccagcacttttatcaactatacgtacaaaaaggtgttgaccaaaaaaataattttttttcttgatcaaagtatgtaaacgcccgcttacagcaaggatcttaagtgagagccattaaattttattgatagctgcttgccaccagtagaatacggccaaaccacctaacaggaaatacaaggcggcccttcggccaataaggtggataaaaatcacgcataagacggttgtaacatagcactttagtgcgaatatcaggaatgccaatagcatgtagataaggcaccaaacatcgcagctatacatggctaaagatcaaccagaaaaggtttaaattttaacgccggcccaaaacttaaactttttttgatatttttaagtgcagccatggattggtccggccataggatgacctatgcctacgtggcattctcattgatggcaatagcaataatatggtatattctacttatctattgccgatcgaaaaaaaatgttgttacaagcggtaatacgctcgctttagcgccaatatcgcatatgtgaaaaatgttcgccgaaaaaaacattaaaatttagaaccgccgcggcatctcaggggcggcaacatttttttttatatggatattgtcacacaccacctcatctatgacgcaatatattactgctaatatcaggttccccaatagtatgtagagaaaccacacaagatagatattcatggcgatttttgacgaaaaaacattaagttttagcttctttgacgcctgtgtactaataatgtttaacgcctgtagtataataattgatacctacagcagtaattgatacctacggcgataatgtctctctggccgccccaaaaaaaagtatttacggtagggtttattaccggcggcgtaacaccagttatggtcaattttgtctggcccgccgcccagccgcaaaaaaaaatcaattacaaccgcaaaaaaaaatatttccggccgcggcgtttcaaaaaataatctttgcgaaataattccgcatcttgtgaaatgaacgcctacagtaataattttaatctttgacacctacagcagtagtaataattttaatctttaacgcctgcagcagtactaatatttttaatgtttaacgcctgcagcagtagtaataattttaatgtttaacgcctacagcagtagtaataattttaatctttgacgcctacagcagtagtaataattttaatgtttaacgcctacagcagtacaataattttaatgtttaacgcctgcagcagtactaatatttttaatctttaacgcctgcagcagtactaatatttttaatctttaacgcctacagcagtagtaataattttaatgtttaacgcctacagcagtagtaataattttaatctttgacgcctacagcagt

(19) United States
(12) Patent Application Publication
(10) Pub. No.: US 2026/0355300 A1
(43) Pub. Date: Nov. 12, 2026

(54) AFRICAN SWINE FEVER VIRUS ASFV-KOR.INJE.MEC-01.2022 ATTENUATED BY CELL ADAPTATION AND USES THEREOF

(71) Applicants: CHOONGANG VACCINE LABORATORIES CO., LTD (CAVAC), Daejeon (KR); REPUBLIC OF KOREA (MINISTRY OF ENVIRONMENT, NATIONAL INSTITUTE OF WILDLIFE DISEASE CONTROL AND PREVE, Gwangju (KR); THE INDUSTRY & ACADEMIC COOPERATION IN CHUNGNAM NATIONAL UNIVERSITY (IAC), Daejeon (KR); GENKORE INC., Daejeon (KR); AVINEXT CO., LTD., Chungcheongbuk-do (KR)

(72) Inventors: IN-JOONG YOON, Daejeon (KR); JOO YOUNG LEE, Daejeon (KR); SUNG-SIK YOO, Daejeon (KR); HYEOK-IL KWON, Daejeon (KR); SEUNG CHUL LEE, Daejeon (KR); MIN HO KIM, Daejeon (KR); WEONHWA JHEONG, Gwangju (KR); YONGKWAN KIM, Gwangju (KR); YEONJI KIM, Gwangju (KR); WONJUN KIM, Gwangju (KR); EUNSOL KIM, Incheon (KR); SANGGEON LEE, Incheon (KR); SOJEONG KIM, Gwangju (KR); GARAM KIM, Gwangju (KR); SONG-I LEE, Gwangju (KR); SEON-MI LEE, Incheon (KR); JAE SUNG PARK, Gwangju (KR); JONG-SOO LEE, Daejeon (KR); INPIL MO, Chungcheongbuk-do (KR); YONG-SAM KIM, Seoul (KR); DO YON KIM, Seoul (KR)

(21) Appl. No.: 19/486,181

(22) PCT Filed: May 27, 2024
(86) PCT No.: PCT/KR2024/007169

§ 371 Date: Nov. 20, 2025

(30) Foreign Application Priority Data
May 26, 2023 (KR) 10-2023-0068629

(51) Int. Cl. A61K 39/12; A61K 39/00; A61P 31/20; C12N 7/00; G01N 33/569

(52) U.S. Cl. CPC A61K 39/12; A61P 31/20; C12N 7/00; G01N 33/56983; A61K 2039/5254; A61K 2039/552; A61K 2039/575; C12N 2710/12021; C12N 2710/12034; C12N 2710/12051; C12N 2710/12064; G01N 2333/01

(57) ABSTRACT

An attenuated African swine fever virus ASFV-KOR.INJE.MEC-01.2022 is obtained by passaging the African swine fever virus (ASFV) in a rhesus monkey kidney cell-derived cell line. The African swine fever virus ASFV-KOR.INJE.MEC-01.2022 naturally attenuated by cell adaptation possesses both safety and efficacy, and therefore it can be effectively utilized as an antigen in vaccine compositions for the prevention of African swine fever.

### REFERENCE TO AN ELECTRONIC SEQUENCE LISTING

A sequence listing electronically submitted on Nov. 20, 2025 as an XML file named 20251120_S65325GR12_TU_SEQ.XML, created on Nov. 20, 2025 and having a size of 181,367 bytes, is incorporated herein by reference in its entirety.

### BACKGROUND

### 1. Technical Field

The present invention relates to African swine fever virus ASFV-KOR.INJE.MEC-01.2022 attenuated by cell adaptation and uses thereof.

This work was supported by Science and Engineering Research Infrastructure Development Project of National Research Foundation of Korea (Project Number: 2021R1A6A1A03045495).

### 2. Background Art

African swine fever (ASF) is a viral disease that primarily infects domestic pigs and wild boars. After the initial infection, symptoms typically appear within 2 to 10 days. Common symptoms include loss of appetite, weakness, reddened skin, inflammation of the conjunctiva, vomiting, bloody diarrhea, and fever. Additionally, the skin may turn blue, necrotic patches (i.e., black spots) may develop, and hemorrhaging may occur. In pregnant sows, ASF can lead to spontaneous abortion. The symptoms of ASF are similar to those of classical swine fever, and depending on the virulence of the virus, the mortality rate among infected animals ranges from 30% to 100%.

The African swine fever virus (ASFV) is a large double-stranded DNA virus belonging to the family Asfarviridae. Its DNA genome varies significantly in length, ranging from 160 to 210 kbp depending on the isolate. The genome contains 150 to 167 open reading frames (ORFs) that encode 54 structural proteins of the ASFV particle and more than 100 infection-related proteins (Dixon et al., 2013. Virus Res 173: 3-14). To date, 24 different genotypes of ASFV have been identified.

As of now, there are no globally developed vaccines or treatments for ASF. ASFV is currently classified into 24 different genotypes, and the greater the variety of virus strains, the more challenging vaccine development becomes. With the recent global rise in African swine fever cases, countries like the United States and China are accelerating vaccine development, but commercialization is expected to take time. Currently, African swine fever has been reported in 53 countries worldwide, including 15 European countries, 29 African countries, and 9 Asian countries. In Asia, where African swine fever is geographically close to South Korea, the disease was first detected in China in August 2018, followed by outbreaks in Mongolia, Vietnam, Cambodia, Laos, Myanmar, the Philippines, and North Korea.

Meanwhile, Korean Patent Publication No. 2021-0065128 discloses an “African swine fever virus vaccine” that relates to a recombinant nucleic acid molecule containing an expression cassette encoding a polyepitope composed of T-cell antigens derived from African swine fever virus proteins. Additionally, Korean Patent Publication No. 2021-0127887 describes polypeptides, polynucleotides, and plasmids involved in generating an immune response against African swine fever and an “African swine fever vaccine composition” containing them. However, there is no disclosure regarding the “African swine fever virus ASFV-KOR.INJE.MEC-01.2022 attenuated by cell adaptation and used thereof” described in the present invention.

### SUMMARY

The present invention is devised in view of the need described above. Specifically, during the process of developing a susceptible cell line for an isolate (wild type) of the African swine fever virus, the inventors of the present invention found that the African swine fever virus, which was being passaged after infecting a cell line derived from rhesus monkey kidney cells, is transformed into an attenuated form, and subsequently found that the naturally attenuated African swine fever virus is safe and effective as a vaccine strain, thereby completing the present invention.

To solve the problems that are described in the above, the present invention provides attenuated African swine fever virus ASFV-KOR.INJE.MEC-01.2022 obtained by passaging the African swine fever virus (ASFV) in a rhesus monkey kidney cell-derived cell line.

The present invention further provides a vaccine composition for prevention of African swine fever, including the attenuated ASFV-KOR.INJE.MEC-01.2022 as an effective component.

The present invention further provides a method for preventing African swine fever by administering the vaccine composition to an animal of the family Suidae.

The present invention further provides a kit for detecting African swine fever virus, which includes the attenuated ASFV-KOR.INJE.MEC-01.2022 as an effective component.

The present invention further provides a method for diagnosing African swine fever infection including reacting the attenuated ASFV-KOR.INJE.MEC-01.2022 with antiserum isolated from a suspected African swine fever-infected individual.

The present invention still further provides a method for producing a vaccine against African swine fever including passaging the attenuated ASFV-KOR.INJE.MEC-01.2022 in a vaccine-producing cell line.

The African swine fever virus ASFV-KOR.INJE.MEC-01.2022 naturally attenuated by cell adaptation according to the present invention possesses both safety and efficacy. Therefore, it can be effectively utilized as an antigen in vaccine compositions for prevention of African swine fever.

### BRIEF DESCRIPTION OF THE DRAWINGS

FIG. 1A shows the results of determining the qPCR Ct value using the ASFV P72 gene to figure out the cell adaptation process of the ASFV isolate according to 18 passages after infecting and passaging a domestic wild boar ASFV isolate (ASF/Korea/Wildbore/Inje-11893/2021) in the CA-CAS-01-A cell line, and FIGS. 1B and 1C show the results of measuring the virus titer of a single vaccine candidate (ASFV-KOR.INJE.MEC-01.2022) of passage 18, which has been finally obtained through plaque assay in CA-CAS-01-A cell line, in which B shows the result of determining TCID₅₀ (tissue culture infectious dose) in CA-CAS-01-A cell line and C shows the result of determining HAD₅₀ (median hemadsorption unit) in primary porcine macrophages.

FIG. 2 illustrates a schematic diagram showing the genomic deletion sites of ASFV-KOR.INJE.MEC-01.2022 (ASFV_MEC-01), produced in CA-CAS-01-A cells infected with the domestic wild boar ASFV isolate (ASF/Korea/Wildbore/Inje-11893/2021).

FIG. 3 illustrates a schematic diagram of the animal test conducted to evaluate the safety and efficacy of the vaccine candidate strain, ASFV-KOR.INJE.MEC-01.2022.

FIG. 4 shows the survival rate of test animals after two doses of the vaccine candidate (ASFV-KOR.INJE.MEC-01.2022) administered at a two-week interval, followed by challenge infection with the ASFV_Hwacheon strain (i.e., domestic wild boar ASFV isolate) and observation for three weeks.

FIG. 5 shows the body temperature changes in test animals following vaccination with the vaccine candidate (ASFV-KOR.INJE.MEC-01.2022) and subsequent challenge infection. The normal temperature range is between 38 °C and 39.5 °C.

FIG. 6 shows the result of measuring the antibody titers in animal test using multi-antigen indirect ELISA analysis for p32, p62, and p72 proteins of ASFV. S/P (sample-to-positive) values are interpreted as follows: ≤30%: Negative, >30% to <40%: Suspected, and ≥40%: Positive

### DETAILED DESCRIPTION

To achieve the object that is described in the above, the present invention provides attenuated African swine fever virus ASFV-KOR.INJE.MEC-01.2022 (hereinbelow, ASFV_MEC-01) obtained by passaging the African swine fever virus (ASFV) in a rhesus monkey kidney cell-derived cell line.

The attenuated African swine fever virus ASFV_MEC-01 of the present invention is characterized by the deletion of genes that are associated with attenuation. Preferably, one or more genes selected from the group consisting of MGF 100-1R, ASFV G ACD 00190, MGF 110-9L, MGF 110-10L, MGF 110-14L, ASFV G ACD 00240, MGF 110-12L, MGF 110-13La, MGF 110-13Lb, ASFV G ACD 00270, MGF 360-4L, MGF 360-6L, MGF 360-8L partial, MGF 300-1L, MGF 300-2R, MGF 300-4L, MGF 110-8L partial, ASFV G ACD 00210, ASFV G ACD 00300, ASFV G ACD 00320, ASFV G ACD 00330, ASFV G ACD 00350, ASFV G ACD 00360 and X69R are deleted. More preferably, all of the following genes may be deleted: MGF 100-1R, ASFV G ACD 00190, MGF 110-9L, MGF 110-10L, MGF 110-14L, ASFV G ACD 00240, MGF 110-12L, MGF 110-13La, MGF 110-13Lb, ASFV G ACD 00270, MGF 360-4L, MGF 360-6L, MGF 360-8L partial, MGF 300-1L, MGF 300-2R, MGF 300-4L, MGF 110-8L partial, ASFV G ACD 00210, ASFV G ACD 00300, ASFV G ACD 00320, ASFV G ACD 00330, ASFV G ACD 00350, ASFV G ACD 00360 and X69R, but the present invention is not limited thereto.

Furthermore, the attenuated African swine fever virus, ASFV_MEC-01 of the present invention may additionally include, based on the ASFV Georgia 2007/1 genome sequence (GenBank: FR682468.2), the following substitutions: the 2,329^{th} base adenine (A) is changed to guanine (G), the 7,059^{th} base cytosine (C) is changed to thymine (T); the 26,425^{th} and 27,200^{th} base thymine (T) is changed to cytosine (C), the 44,576^{th} base adenine (A) is changed to guanine (G); the 72,295^{th}, 72,315^{th}, and 72,316^{th} base guanine (G) are changed to adenine (A), and cytosine (C), which is the 179,097^{th}, 180,319^{th}, 180,607^{th}, 180,619^{th}, 180,718^{th}, 181,034^{th}, 181,322^{nd}, 181,809^{th}, 181,891^{st} and 182,053^{rd} base, is changed to thymine (T) (see Table 6); and the above base mutations may be the following: the 106^{th} amino acid leucine (Leu, L) of the MGF 360-1La protein is changed to proline (Pro, P), the 197^{th} amino acid tryptophan (Trp, W) of the MGF 110-1L protein is changed to a stop codon, the 69^{th} and 329^{th} amino acids asparagine (Asn, N) of the MGF360-10L protein is changed to serine (Ser, S), the 323^{rd} amino acid lysine (Lys, K) of the MGF 505-9R protein is changed to glutamic acid (Glu, E), and the 14^{th} and 422^{nd} amino acids methionine (Met, M) and alanine (Ala, A) of the MGF505-11L protein are changed to isoleucine (Ile, I) and threonine (Thr, T), respectively; the 96^{th} and 100^{th} amino acids aspartic acid (Asp, D) and glutamic acid (E) of the MGF 100-1L protein are changed to asparagine (N) and lysine (K), respectively, and the 86^{th} amino acid alanine (A) of the MGF 100-3L protein is changed to threonine (T), but the present invention is not limited thereto.

In one embodiment of the present invention, the genome sequence of the attenuated African swine fever virus ASFV_MEC-01 may be composed of the nucleotide sequence of SEQ ID NO: 3.

The attenuated ASFV_MEC-01 of the present invention is characterized by having a viral titer of at least 1×10⁸ HAD₅₀/mL in primary porcine macrophages.

Additionally, in the present invention, the rhesus monkey kidney cell-derived cell line may be the CA-CAS-01-A cell line, which has the deposit number KCTC 14568BP. The aforementioned CA-CAS-01-A cell line was duly deposited with Korean Collection for Type Cultures (KCTC) (having the address of Korea Research Institute of Biosciences and Biotechnology (KRIBB) 181, Ipsin-gil, Jeongeup-si, Jeollabuk-do 56212, Republic of Korea) under the Access number of KCTC14568BP on May 12, 2021. The deposit has been made under the terms of the Budapest Treaty and all restrictions imposed by the depositor on the availability to the public of the biological material will be irrevocably removed upon the granting of a patent.

The present invention further provides a vaccine composition for prevention of African swine fever, including the attenuated ASFV_MEC-01 as an effective component.

In the vaccine composition of the present invention, the attenuated African swine fever virus ASFV_MEC-01 is as described above and has a genome consisting of the nucleotide sequence of SEQ ID NO: 3.

Form of the vaccine composition according to the present invention may be selected from the group consisting of a live vaccine, an inactivated vaccine, a subunit vaccine produced using a gene of an attenuated African swine fever virus, a vector vaccine, a chimeric vaccine, a DNA and RNA vaccine, but is not limited thereto.

In the present invention, the term “prevention” means any act of suppressing or delaying the occurrence, spread, and recurrence of African swine fever infection by administering the vaccine composition of the present invention.

In addition, the vaccine composition of the present invention may additionally include a pharmaceutically acceptable carrier and/or adjuvant. In addition to the carrier, the vaccine composition of the present invention may additionally include an excipient and/or a diluent.

The term “pharmaceutically acceptable” as used herein refers to a substance that is physiologically tolerable and does not typically cause allergic reactions such as gastrointestinal upset, dizziness or the like when administered to a mammal Examples of such carriers, excipients and diluents include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil. Additionally, it may contain fillers, anticoagulants, lubricants, wetting agents, fragrances, emulsifiers, and preservatives.

In addition, the “adjuvant” as used herein means a pharmaceutical or immunological agent administered for the purpose of enhancing the immune response of a vaccine. As the adjuvant, aluminum hydroxide, aluminum phosphate, alum (potassium aluminum sulfate), MF59, virosome, AS04 [a mixture of aluminum hydroxide and monophosphoryl lipid A (MPL)], AS03 (a mixture of DL-α-tocopherol, squalene, and polysorbate 80, an emulsifier), CpG, Flagellin, Poly I:C, AS01, AS02, ISCOMs, or ISCOMMATRIX can be used.

The vaccine composition of the present invention can be formulated using methods known in the pertinent art to enable rapid release, or sustained or delayed release, of the effective component when administered to a mammal. The formulations include powders, granules, tablets, emulsions, syrups, aerosols, soft or hard gelatin capsules, sterile injectable solutions, and sterile powders.

The vaccine composition according to the present invention can be administered by various routes, for example, orally, parenterally, for example, suppository, transdermal, intravenous, intraperitoneal, intramuscular, intralesional, nasal, intrathecal administration, and also can be administered using an implantable device for sustained release or continuous or repeated release. The number of administrations can be once a day or divided into several times within a desired range, and the administration period is not particularly limited.

The present invention further provides a method for preventing African swine fever by administering the vaccine composition to an animal of the family Suidae.

In the method for preventing African swine fever according to the present invention, the animal of the family Suidae may preferably be a wild boar (genus Sus) animal to which a domesticated species (Sus scrofa) belongs, but is not limited thereto.

The present invention further provides a kit for detecting African swine fever virus, which includes the attenuated ASFV_MEC-01 as an effective component.

The kit may include a reagent capable of detecting the attenuated African swine fever virus ASFV_MEC-01 according to the present invention and an antibody specifically binding to the attenuated African swine fever virus ASFV_MEC-01. In the kit of the present invention, the attenuated African swine fever virus ASFV_MEC-01 functions as an antigen, and the reagent for detecting the virus (antigen)-antibody complex may include, but is not limited to, a reagent for radial immunoassay, ELISA (Enzyme linked immunosorbent assay), or immunofluorescence analysis.

The present invention further provides a method for diagnosing African swine fever infection including reacting the attenuated ASFV_MEC-01 with antiserum isolated from a suspected African swine fever-infected individual.

The term “individual” used in the present invention means a mammal that has developed or can develop African swine fever infection, and may preferably include a pig or a wild boar.

The method for diagnosing African swine fever infection according to the present invention may consist of a step of reacting a sample (i.e., antiserum) isolated from a suspected African swine fever individual with the African swine fever virus ASFV_MEC-01 of the present invention under conditions in which an antigen/antibody complex can be formed, and then detecting the formation of the antigen/antibody complex. The method for detecting the formation of the antigen/antibody complex can be performed through a method that is well known in the pertinent art.

The present invention still further provides a method for producing a vaccine against African swine fever including passaging the attenuated ASFV_MEC-01 in a vaccine-producing cell line.

In the production method of the present invention, the vaccine-producing cell line may preferably be the cell line CA-CAS-01-A deposited with the deposit number KCTC14568BP, but is not limited thereto, and there is no limitation on the type of cell as long as it is a cell in which infection and proliferation of the attenuated African swine fever virus ASFV_MEC-01 of the present invention can occur.

In the production method of the present invention, the attenuated African swine fever virus ASFV_MEC-01 or the antigens derived therefrom can be converted into a physiologically acceptable form. This process can be carried out based on experience in the production of a vaccine against influenza. For example, to prepare a vaccine injection, virus particles can be lyophilized (i.e., freeze-dried) in 100 mL of phosphate-buffered saline (PBS) in the presence of 1% human albumin and 2% peptone in an ampoule, preferably in a glass ampoule. Alternatively, the vaccine injection may be produced by sequential freeze-drying of the virus in the preparation. The preparation may contain additional additives such as mannitol, dextran, sugars, glycine, lactose or polyvinylpyrrolidone or other auxiliaries such as antioxidants or inert gases, stabilizers or recombinant proteins suitable for in vivo administration (e.g. human serum albumin). The glass ampoules are then sealed and can be stored for several months between 4 °C and room temperature. However, unless otherwise required, the ampoules can preferably be stored at below −20 °C.

Hereinbelow, the present invention is explained in greater detail in view of Examples. However, the following Examples are given only for exemplification of the present invention and it is evident that the scope of the present invention is not limited by them.

### EXAMPLES

### Materials and Methods

### 1. Cell and Virus

Monkey kidney cell-derived CA-CAS-01-A cell line (deposit number: KCTC14568BP) was maintained in α-MEM (Invitrogen) medium containing 5% fetal bovine serum (FBS; Invitrogen) and antibacterial and antifungal solution (100×; Invitrogen) at 37 °C in a humidified 5% CO₂ incubator under passaging every 2 to 3 days. As for the African swine fever virus (ASFV), a domestic isolate (ASF/Korea/Wildbore/Inje-11893/2021) isolated from a wild boar, which had occurred in Inje, Gangwon-do, South Korea in 2022, and provided by the Korean National Institute of Wildlife Disease Management, was used.

### 2. Virus Passaging

ASFV domestic isolate (ASF/Korea/Wildbore/Inje-11893/2021) was adapted to the CA-CAS-01-A cell line, and qPCR (quantitative PCR) was performed using a primer set specific for the ASFV P72 gene (Table 1). Then, ASFV culture with a Ct value of 22 or less (Ct value: approximately 18) was used in the experiment.

A serum-free medium was used as a medium for ASFV infection, and 3 to 4 days after the infection, it was replaced with the virus growth medium (i.e., serum-free medium) used for infection, and the previously sensitized virus was removed to determine the propagated virus. Passaging was performed at 7-day intervals after the infection, and the culture broth of the 1st, 3rd, 5th, and 10th passages were collected, the supernatants and cells were frozen at −80 °C, and then thawed once in a 37 °C constant temperature water bath. qPCR was then performed using the culture broth to measure the Ct value, and a test to determine intracytoplasmic infection (Alkaline Phosphatase assay_ASFV P30 detection; 1st antisera: Rabbit anti African swine Fever virus phosphoprotein p30 antisera, 2nd antibody: Goat Anti-rabbit IgG H&L, Alkaline phosphate substrate kit: ImmPACT Vector RED) was carried out. When it is found to be ASFV-positive, 2 mL of the first-passage culture broth (1×10⁶ CFU/mL) was used along with 8 mL of serum-free medium for the next passage. This process was repeated for subsequent passages.

TABLE 1
Information of primers used for qPCR

| Primer Name | Nucleotide Sequence (5′→3′) | SEQ ID NO: |
|---|---|---|
| P72_Forward | CTG CTC ATG GTA TCA ATC TTA TCG A | 1 |
| P72_Reverse | GAT ACC ACA AGA TC(AG) GCC GT | 2 |

### 3. Virus Titer Measurement

From CA-CAS-01-A cell line infected with the ASFV isolate, culture supernatant was collected at the time point when 70% CPE (cytopathic effect) occurred after the infection. For growth kinetics experiment, the supernatant was collected at different time points from cells infected with each virus strain, and then stored at −80 °C. Virus titer was examined by quantitative real-time RT-PCR using the primer sets described in above Table 1. Quantitative real-time RT-PCR employed the following conditions: 1 cycle of 50 °C for 2 minutes and 95 °C for 10 minutes for the pretreatment process, and 40 cycles of 95 °C for 15 seconds and 60 °C for 1 minute for the main process.

### 4. Nucleotide Sequencing

The whole-length nucleotide sequence of the cell-adapted ASFV was analyzed by Celemics, Inc. upon request.

### 5. Determination of Antibody Titer (ELISA)

ASF virus P32, P62, and P72 were detected by ELISA and antibody titers were measured. The measurement was performed using the ID SCREENA® African Swine Fever Indirect (ASF Elisa) kit (IDvet). Briefly, blood collected from the vaccinated group and negative group of the test animals was centrifuged at 4,500 rpm for 10 minutes, and the supernatant (serum) was collected 190 μl of dilution buffer were added to each well, 10 μl of the negative control group were added to wells A1 and B1, and 10 μl of the positive control group were added to wells C1 and D1. The serum samples collected above were added to each of the remaining wells, and then reacted at 21 °C (±5) for 45 minutes (±4 minutes). After that, the reactants in each well were removed, washed three times with 300 μl of wash buffer, and 100 μl of concentrated conjugate which has been diluted 1/10 using dilution buffer were added to each well and reacted at 21 °C (±5) for 30 minutes (±3 minutes). After removing the reactants in each well, washing was carried out three times with 300 μl of wash buffer, 100 μl of substrate solution were added to each well, and the reaction was allowed to occur for 15 minutes (±2 minutes) in the dark at 21 °C (±5). Thereafter, 100 μl of reaction stop solution were added and the absorbance was measured at 450 nm.

The validity of the reaction results is determined based on the following criteria:
Average OD value of the positive control is greater than 0.350 (ODPC>0.350).
Ratio of the average OD value of the positive control to the average OD value of the negative control is greater than 3 (ODPC/ODNC>3).
Sample-to-positive percentage (S/P %) for each sample is calculated using the following formula:

S/P %={(ODsample−ODNC)/(ODPC−ODNC)}×100,
in which interpretation of S/P % values is made as follows:
S/P %≤30%→Negative
30%<S/P %<40%→Suspicious
S/P %≥40%→Positive.

### 6. Pig Infection Test

In the present invention, in vivo pig studies were conducted in accordance with the guidelines of the Institutional Animal Care and Use Committee (IACUC), and the tests were carried out at a biosafety level 3 (BSL-3) research facility within the Wildlife Disease Control Center of the Ministry of Environment, Korea.

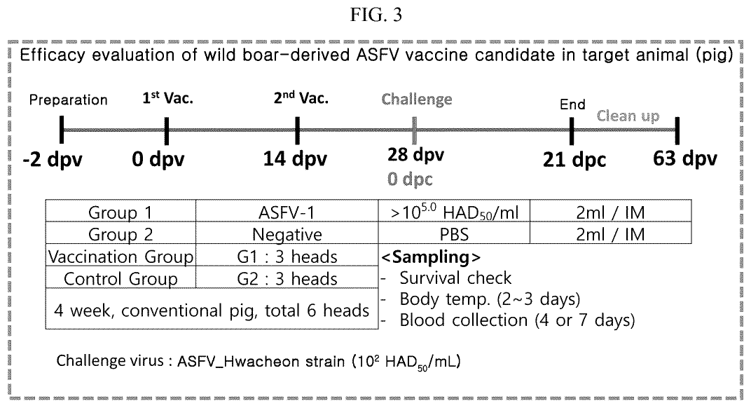

The inventors of the present invention conducted animal test to evaluate the attenuation (safety) and vaccine efficacy of the 18th-passaged attenuated ASF virus (ASFV-KOR.INJE.MEC-01.2022; ASFV_MEC-01), which was propagated and attenuated using the CA-CAS-01-A cell line. Briefly, 3- to 4-week-old pigs were intramuscularly injected with the vaccine candidate at a dose of >10⁵ HAD₅₀/mL, administered twice in total at a 2-week interval. The pigs were then challenged with ASFV_Hwacheon strain (10² HAD₅₀/mL) (FIG. 3). To monitor the detection level of virus, oral swabs, rectal swabs, nasal swabs, serum, and whole blood samples were collected every 2 to 3 days. Additionally, temperature changes, clinical symptoms, and survival rates were observed over a period of 3 weeks.

### 7. Statistical Analysis

The Student's t-test was used for all statistical analyses, and a P-value of less than 0.05 was considered statistically significant.

### Example 1. Characterization of Cell-Adapted ASFV Isolate

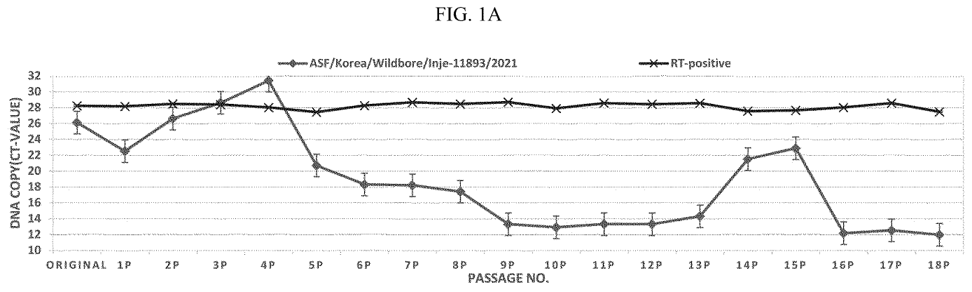

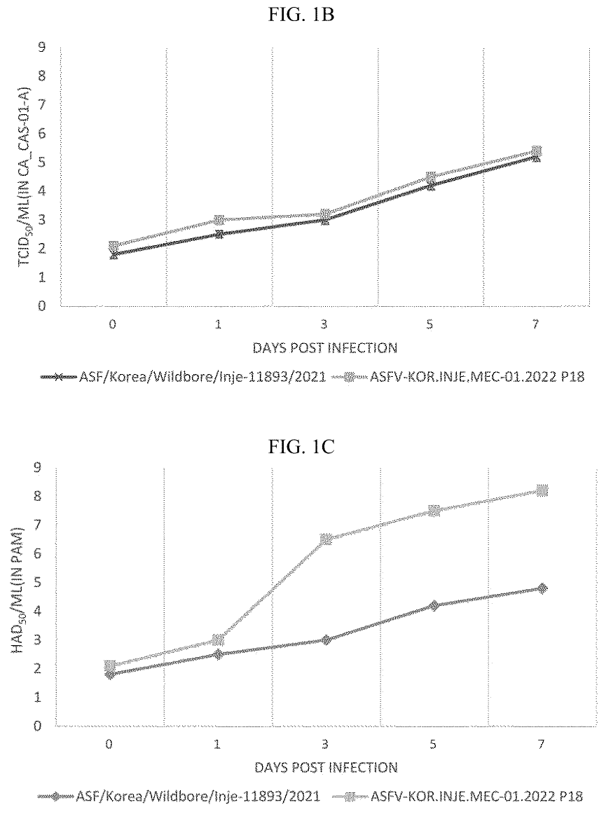

The ASFV strain isolated from wild boars in Korea (ASF/Korea/Wildbore/Inje-11893/2021) was infected into the CA-CAS-01-A cell line (deposit number: KCTC14568BP) and subjected to serial passage culture. The adaptation process of the ASFV isolate in cells was then analyzed using qPCR targeting the ASFV P72 gene. As a result, as shown in FIG. 1A, the Ct value began to drop below 20 after the 5th passage, and a Ct value of approximately 12 or lower was observed after the 16th passage. Additionally, the virus titer of the 18th-passage single vaccine candidate strain (ASFV_MEC-01), which was finally obtained through plaque assay in CA-CAS-01-A cells, is presented in Table 2 below.

TABLE 2
Results of titer determination of vaccine
candidate (ASFV-KOR.INJE.MEC-01.2022)

Titration (7 dpi)

| | | HAD₅₀/mL | |
|---|---|---|---|
| | CT-value | (Primary | TCID₅₀/mL |
| | (CA_CAS- | swine | (CA_CAS- |
| ASFV Virus | 01_A) | macrophage) | 01_A) |
| ASFV- | 11.97 | 1.122 × 10⁸ | 3.981 × 10⁵ |
| KOR.INJE.MEC- | | | |
| 01.2022 | | | |

Additionally, the ASFV isolate (ASFV_MEC-01) passaged 18 times in total was infected into either the CA-CAS-01-A cell line or primary porcine macrophages, and its viral propagation over time was assessed using either TCID₅₀ (tissue culture infectious dose) or HAD₅₀ (median hemadsorption unit). The results indicated that even after adaptation passage in the CA-CAS-01-A cell line, the viral propagation capacity did not decrease compared to the original isolate—in fact, it showed a slight increase (FIGS. 1B and 1C).

### Example 2. Genomic Analysis of Cell-Adapted ASFV Isolate

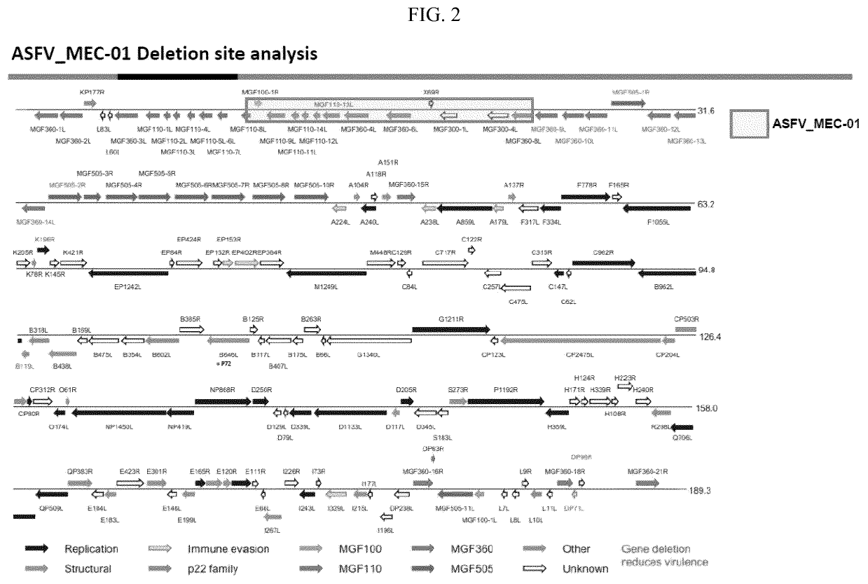

In order to determine the characteristics of the final secured vaccine candidate (ASFV_MEC-01), the virus was recovered from the culture broth after passaging for 18 times and its genome sequence was analyzed. As a result, it was found that a large gene deletion occurred in the 5′ region, as illustrated in FIG. 2. The genes located in the deleted region are summarized in Table 3, and, among the deleted genes, the genes associated with the attenuation are as described in Table 4. In addition, the positions of the deleted genes in Table 4 compared to the sequence of African swine fever virus isolate ASFV Georgia 2007/1 genome assembly, complete genome: monopartite (GenBank: FR682468.2) are as described in Table 5.

TABLE 3
Genes in ASFV-KOR.INJE.MEC-01.2022 (ASFV_MEC-01) deletion site

| | | NGS | PCR status |
|---|---|---|---|
| MGF 100 | IL | Gene | |
| | 1R | Deletion | Not detected |
| | 3L | Gene | |
| MGF 110 | 1L | Gene | |
| | 2L | Gene | |
| | 3L | Gene | |
| | 4L | Gene | |
| | 5L-6L | Gene | |
| | 7L | Gene | |
| | 8L | Deletion (N terminal 80%) | |
| | 9L | Deletion | |
| | 10L | Deletion | |
| | 14L | Deletion | |
| | 12L | Deletion | |
| | 13La | Deletion | |
| | 13Lb | Deletion | |
| MGF 300 | 1L | Deletion | |
| | 2R | Deletion | Not detected |
| | 4L | Deletion | Not detected |
| MGF 360 | 1La | Gene | |
| | 1Lb | Gene | |
| | 2L | Gene | |
| | 3L | Gene | |
| | 4L | Deletion | Not detected |
| | 6L | Deletion | Not detected |
| | 8L | Deletion (C terminal Frameshift 50%) | Not detected |
| | 9L | Gene | |
| | 10L | Gene | |
| | 11L | Gene | |
| | 12L | Gene | |
| | 13L | Gene | |
| | 14L | Deletion (C terminal 20%) | |
| | 15R | Gene | |
| | 16R | Gene | |
| | 18R | Gene | |
| | 19Ra | Gene | |
| | 19Rb | Gene | |
| | 21R | Deletion (C terminal AA deletion) | PCR - No deletion |
| G_ACD | G ACD 00120 | Gene | |
| | G ACD 00160 | Gene | |
| | G ACD 00190 | Deletion | |
| | G ACD 00210 | | |
| | G ACD 00240 | | |
| | G ACD 00270 | | |
| | G ACD00300 | | |
| | G ACD00320 | | |
| | G ACD00330 | | |
| | G ACD00350 | | |
| | G ACD00360 | | |
| | G ACD00520 | Gene | |
| Other | X69R | Deletion | |
| | 285L | Gene | |
| | I7L | Gene | |
| | I8L | Gene | |
| | I9R | Gene | |
| | I10L | Deletion (N terminal 1^{st} 4 to 6 AA) | PCR - No deletion |
| | L11L | Gene | |
| | A224L | Deletion (N terminal 1^{st} 6 to 10 AA) | |
| | EP153R | Deletion (C terminal- last 4 AA) | PCR - No deletion |
| | EP402R | Deletion (N terminal 7.5%) | PCR - No deletion |
| | NP868R | Gene | |
| | P1192R | Gene | |
| | E199L | Gene | |
| | EP424R | Gene | |
| | C962R | Deletion | |
| | | (C terminal frameshift 60%) | |
| | NP419L | Gene | |
| | I267L | Gene | |
| | I329L | Deletion (N terminal 1^{st} 8-10 AA) | PCR - No deletion |
| | I177L | Deletion (N terminal frameshift 92%) | PCR - No deletion |

TABLE 4
Results of genomic analysis of attenuated ASFV_MEC-01 virus

| | Gene deleted |
|---|---|
| Deletion site | MGF 100-1R, ASFV G ACD 00190, MGF 110-9L, MGF 110-10L, MGF 110- |
| | 14L, ASFV G ACD 00240, MGF 110-12L, MGF 110-13La, MGF 110-13Lb, |
| | ASFV G ACD 00270, MGF 360-4L, MGF 360-6L, MGF 360-8L partial, |
| | MGF 300-1L, MGF 300-2R, MGF 300-4L, MGF 110-8L partial, ASFV G |
| | ACD 00210, ASFV G ACD 00300, ASFV G ACD 00320, ASFV G ACD 00330, |
| | ASFV G ACD 00350, ASFV G ACD 00360, X69R |

Bold: Genes affecting attenuation

TABLE 5
Site of gene deletion

| ASFV_MEC-01 | | | | Passage 18 |
|---|---|---|---|---|
| Deletion size | | | | 11,527~24,491 |
| Deletion gene | MGF 100 | 1R | 12056 . . . 12430 | Del |
| | MGF 110 | 8L | 11455 . . . >11838 | Partial |
| | | 9L | 12590 . . . 13462 | Del |
| | | 10L-14L fusion | 13753 . . . 14571 | Del |
| | | 12L | 14761 . . . 15120 | Del |
| | | 13La | 15206 . . . 15553 | Del |
| | | 13Lb | 15663 . . . 16031 | Del |
| | MGF 300 | 1L | 20704 . . . 21510 | Del |
| | | 2R | 22336 . . . 22818 | Del |
| | | 4L | 22908 . . . 23900 | Del |
| | MGF 360 | 4L | 16212 . . . 17375 | Del |
| | | 6L | 18189 . . . 19316 | Del |
| | | 8L | 24014 . . . 24973 | Partial |
| | G_ACD | G ACD 00190 | 12456 . . . 12581 | Del |
| | | G ACD 00210 | 13461 . . . 13652 | Del |
| | | G ACD 00240 | 14570 . . . 14680 | Del |
| | | G ACD 00270 | 16056 . . . 16169 | Del |
| | | G ACD 00300 | 17929 . . . 18045 | Del |
| | | G ACD 00320 | 19544 . . . 19669 | Del |
| | | G ACD 00330 | 19857 . . . 19970 | Del |
| | | G ACD 00350 | 19967 . . . 20098 | Del |
| | | G ACD 00360 | 20169 . . . 20285 | Del |
| | Other | X69R | 20197 . . . 20406 | Del |

* Reference sequence: ASFV Georgia 2007/1 genome (GenBank: FR682468.2)

Furthermore, in addition to the above gene deletions, many specific nucleotide mutations were identified in the genome sequence of the vaccine candidate ASFV_MEC-01 compared to the reference sequence (ASFV Georgia 2007/1 genome, GenBank: FR682468.2) and the genome sequence of the isolate (ASF/Korea/Wildbore/Inje-11893/2021).

TABLE 6
Results of genomic analysis of ASFV-KOR.INJE.MEC-01.2022 (ASFV_MEC-01) - Point mutation

| | Gene | MGF360-1La | MGF110-1L | MGF 360-10L | | MGF 505-9R | EP424R | | | MGF505-11L |
|---|---|---|---|---|---|---|---|---|---|---|
| | Function | Unknown | non-essential | Partial attenuated | | | mRNA modification | | | replication |
| | | | | | | | | | | in macrophages |
| | Necleotide Position | 2,329 | 7,059 | 26,425 | 27,200 | 44,576 | 72,295 | 72,315 | 72,316 | 179,097 |
| | Amino acid change | L106P | W197* | N69S | N329S | K323E | silent mutation | | | M14I |
| Virus | Georgia/2007/1 | A | C | T | T | A | G | G | G | C |
| | ASF/Korea/Wildbore/ | A | C | T | T | A | G | G | G | C |
| | Inje-11893/2021 | | | | | | | | | |
| | ASFV-KOR.INJE.MEC- | G | T | C | C | G | A | A | A | T |
| | 01.2022 | | | | | | | | | |
| | Gene | MGF505-11L | MGF100-1L | | | NCR | MGF100-3L | I7L | | hypthetical |
| | Function | replication | specific CD8+ | | | Unknown | Unknown | Partial virulence | | Unknown |
| | | in macrophages | T-cell epitopes | | | | | | | |
| | Necleotide Position | 180,319 | 180,607 | 180,619 | 180,718 | 181,034 | 181,322 | 181,809 | 181,891 | 182,053 |
| | Amino acid change | A422T | D96N | E100K | silent mutation | | A86T | silent mutation | | |
| Virus | Georgia/2007/1 | C | C | C | C | C | C | C | C | C |
| | ASF/Korea/Wildbore/ | C | C | C | C | C | C | C | C | C |
| | Inje-11893/2021 | | | | | | | | | |
| | ASFV-KOR.INJE.MEC- | T | T | T | T | T | T | T | T | T |
| | 01.2022 | | | | | | | | | |

The vaccine candidate ASFV_MEC-01 of the present invention showed genetic stability (in terms of deleted site and location of point mutation) up to 20 passages.

### Example 3. Safety and Efficacy Analysis of Vaccine Candidates

To evaluate the safety (attenuation) and efficacy of the final vaccine candidate ASFV_MEC-01, animal tests were conducted.

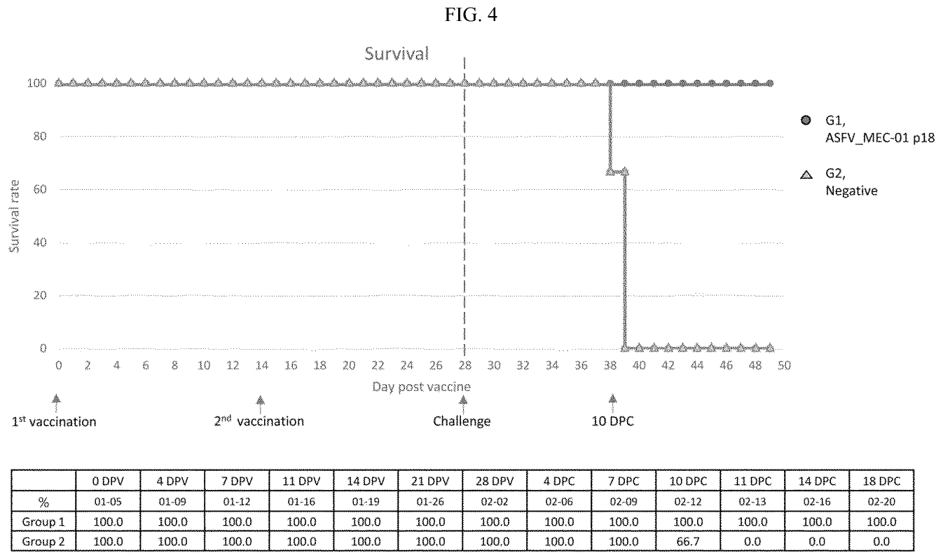

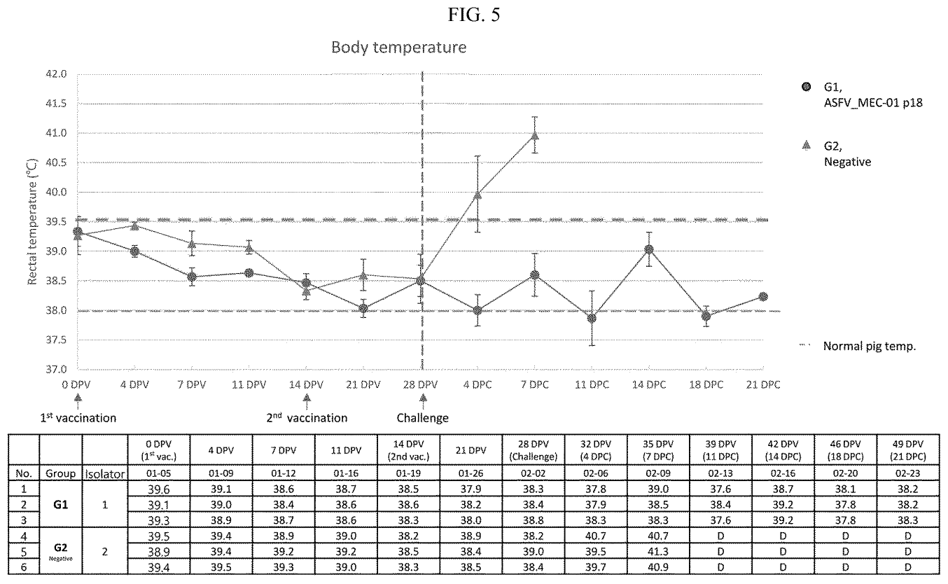

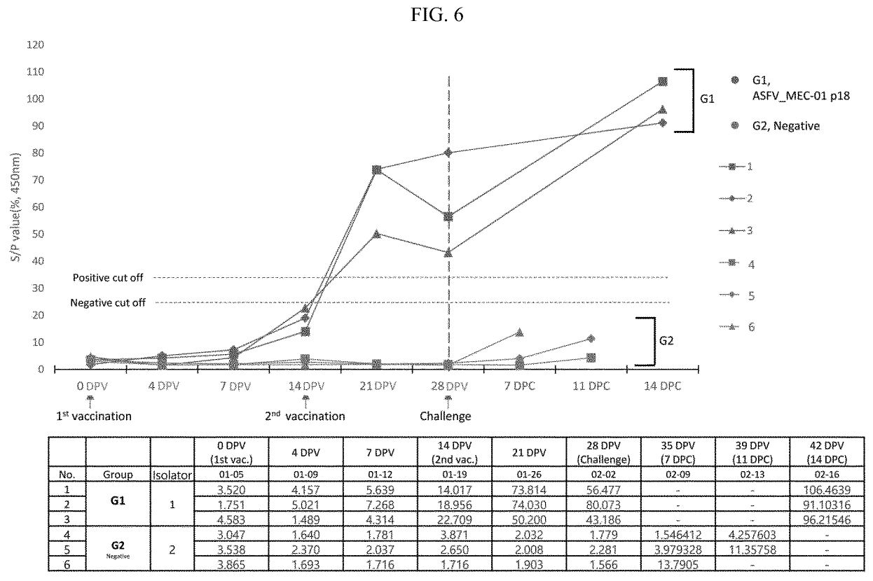

The vaccine candidate (ASFV_MEC-01) was administered twice at two-week intervals, followed by a challenge infection with ASFV_Hwacheon strain (isolated from wild boars in Korea). The three-week survival rate of the test animals was then assessed. The results showed that in the non-immunized group (i.e., not immunized with any vaccine candidate), mortality began on day 10 post-challenge, and all animals died by day 11. In contrast, the group immunized with the vaccine candidate demonstrated a 100% survival rate (FIG. 4). Additionally, the group immunized with the vaccine candidate maintained normal body temperature throughout the vaccination period and post-challenge, showing no fever symptoms. However, in the non-vaccinated group, fever symptoms appeared from day 4 post-challenge (FIG. 5). Furthermore, multi-antigen indirect ELISA analysis for ASFV p32, p62, and p72 proteins revealed that antibody titers gradually increased after the second vaccine candidate dose, turning seropositive within a week after the second dose of the vaccine. The antibody level remained high up to 14 days post-challenge (FIG. 6). This strong antibody titer is believed to have contributed to protection against the ASFV_Hwacheon strain.

Based on the above results, it was found that, as the ASF vaccine candidate ASFV-KOR.INJE.MEC-01.2022 (ASFV_MEC-01) demonstrates both safety through natural attenuation and efficacy as a vaccine, it can be utilized as a valuable antigen for the development of an effective ASF vaccine.

1: An attenuated African swine fever virus ASFV-KOR.INJE.MEC-01.2022 obtained by passaging African swine fever virus (ASFV) in a rhesus monkey kidney cell-derived cell line.

2: The attenuated African swine fever virus ASFV-KOR.INJE.MEC-01.2022 according to claim 1, wherein the rhesus monkey kidney cell-derived cell line is CA-CAS-01-A cell line deposited with deposit number KCTC 14568BP.

3: The attenuated African swine fever virus ASFV-KOR.INJE.MEC-01.2022 according to claim 1, wherein the attenuated African swine fever virus has deletion of one or more genes selected from the group consisting of MGF 100-1R, ASFV G ACD 00190, MGF 110-9L, MGF 110-10L, MGF 110-14L, ASFV G ACD 00240, MGF 110-12L, MGF 110-13La, MGF 110-13Lb, ASFV G ACD 00270, MGF 360-4L, MGF 360-6L, MGF 360-8L partial, MGF 300-1L, MGF 300-29 MGF 300-4L, MGF 110-8L partial, ASFV G ACD 00210, ASFV G ACD 00300, ASFV G ACD 00320, ASFV G ACD 00330, ASFV G ACD 00350, ASFV G ACD 00360 and X69R.

4: The attenuated African swine fever virus ASFV-KOR.INJE.MEC-01.2022 according to claim 3, wherein the attenuated African swine fever virus has deletion of MGF 100-1R, ASFV G ACD 00190, MGF 110-9L, MGF 110-10L, MGF 110-14L, ASFV G ACD 00240, MGF 110-12L, MGF 110-13La, MGF 110-13Lb, ASFV G ACD 00270, MGF 360-4L, MGF 360-6L, MGF 360-8L partial, MGF 300-L, MGF 300-29 MGF 300-4L, MGF 110-8L partial, ASFV G ACD 00210, ASFV G ACD 00300, ASFV G ACD 00320, ASFV G ACD 00330, ASFV G ACD 00350, ASFV G ACD 00360 and X69R genes.

5: The attenuated African swine fever virus ASFV-KOR.INJE.MEC-01.2022 according to claim 4, wherein the attenuated African swine fever virus additionally comprises any of the following mutations: based on ASFV Georgia 2007/1 genome sequence, the 2,329^{th} base adenine (A) is changed to guanine (G), the 7,059^{th} base cytosine (C) is changed to thymine (T); the 26,425^{th} and 27,200^{th} base thymine (T) is changed to cytosine (C), the 44,576^{th} base adenine (A) is changed to guanine (G); the 72,295^{th}, 72,315^{th}, and 72,316^{th} base guanine (G) is changed to adenine (A), and cytosine (C), which is the 179,097^{th}, 180,319^{th}, 180,607^{th}, 180,619^{th}, 180,718^{th}, 181,034^{th}, 181,322^{nd}, 181,809^{th}, 181,891^{st} and 182,053^{rd} base, is changed to thymine (T).

6: A vaccine composition for prevention of African swine fever, comprising the attenuated African swine fever virus ASFV-KOR.INJE.MEC-01.2022 of claim 1 as an effective component.

7: The vaccine composition according to claim 6, wherein the vaccine composition additionally comprises a pharmaceutically acceptable carrier or adjuvant.

8: A method for preventing African swine fever, the method comprising:
administering the vaccine composition of claim 6 to an animal of family Suidae.

9: A kit for detecting African swine fever virus, which comprises the attenuated African swine fever virus ASFV-KOR.INJE.MEC-01.2022 of claim 1 as an effective component.

10: A method for diagnosing African swine fever infection, the method comprising:
reacting the attenuated African swine fever virus ASFV-KOR.INJE.MEC-01.2022 of claim 1 with antiserum isolated from a suspected African swine fever-infected individual.

11: A method for producing a vaccine against African swine fever, the method comprising:
passaging the attenuated African swine fever virus ASFV-KOR.INJE.MEC-01.2022 of claim 1 in a vaccine-producing cell line.
